SEQUENCE LISTING (199 sequences)

SEQ ID NO: 1 - protein, 15 aa, Artificial Sequence
  CD19 VL CDR1
KASQSVDYDGDSYLN

SEQ ID NO: 2 - protein, 7 aa, Artificial Sequence
  CD19 VL CDR2
DASNLVS

SEQ ID NO: 3 - protein, 9 aa, Artificial Sequence
  CD19 VL CDR3
QQSTEDPWT

SEQ ID NO: 4 - protein, 5 aa, Artificial Sequence
  CD19 VH CDR1
SYWMN

SEQ ID NO: 5 - protein, 17 aa, Artificial Sequence
  CD19 VH CDR2
QIWPGDGDTNYNGKFKG

SEQ ID NO: 6 - protein, 15 aa, Artificial Sequence
  CD19 VH CDR3
RETTTVGRYYYAMDY

SEQ ID NO: 7 - protein, 111 aa, Artificial Sequence
  CD19 VL
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIK

SEQ ID NO: 8 - protein, 124 aa, Artificial Sequence
  CD19 VH
QVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSS

SEQ ID NO: 9 - protein, 5 aa, Artificial Sequence
  CD3 VH CDR1
RYTMH

SEQ ID NO: 10 - protein, 17 aa, Artificial Sequence
  CD3 VH CDR2
YINPSRGYTNYNQKFKD

SEQ ID NO: 11 - protein, 10 aa, Artificial Sequence
  CD3 VH CDR3
YYDDHYCLDY

SEQ ID NO: 12 - protein, 10 aa, Artificial Sequence
  CD3 VL CDR1
RASSSVSYMN

SEQ ID NO: 13 - protein, 7 aa, Artificial Sequence
  CD3 VL CDR2
DTSKVAS

SEQ ID NO: 14 - protein, 9 aa, Artificial Sequence
  CD3 VL CDR3
QQWSSNPLT

SEQ ID NO: 15 - protein, 119 aa, Artificial Sequence
  CD3 VH
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSS

SEQ ID NO: 16 - protein, 108 aa, Artificial Sequence
  CD3 VL
VDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 17 - protein, 504 aa, Artificial Sequence
  CD19xCD3 scFv BLINCYTO incl linker and his-tag
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKHHHHHH

SEQ ID NO: 18 - protein, 14 aa, Artificial Sequence
  CDR-L1 I2C
GSSTGAVTSGNYPN

SEQ ID NO: 19 - protein, 7 aa, Artificial Sequence
  CDR-L2 I2C
GTKFLAP

SEQ ID NO: 20 - protein, 9 aa, Artificial Sequence
  CDR-L3 I2C
VLWYSNRWV

SEQ ID NO: 21 - protein, 5 aa, Artificial Sequence
  CDR-H1 I2C
KYAMN

SEQ ID NO: 22 - protein, 19 aa, Artificial Sequence
  CDR-H2 I2C
RIRSKYNNYATYYADSVKD

SEQ ID NO: 23 - protein, 14 aa, Artificial Sequence
  CDR-H3 I2C
HGNFGNSYISYWAY

SEQ ID NO: 24 - protein, 125 aa, Artificial Sequence
  VH I2C
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSS

SEQ ID NO: 25 - protein, 109 aa, Artificial Sequence
  VL I2C
QTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 26 - protein, 249 aa, Artificial Sequence
  VH-VL I2C
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 27 - protein, 122 aa, Artificial Sequence
  CD33 ccVH E11
QVQLVQSGAEVKKPGESVKVSCKASGYTFTNYGMNWVKQAPGQCLEWMGWINTYTGEPTYADKFQGRVTMTTDTSTSTAYMEIRNLGGDDTAVYYCARWSWSDGYYVYFDYWGQGTSVTVSS

SEQ ID NO: 28 - protein, 122 aa, Artificial Sequence
  CD33 VH E11
QVQLVQSGAEVKKPGESVKVSCKASGYTFTNYGMNWVKQAPGQGLEWMGWINTYTGEPTYADKFQGRVTMTTDTSTSTAYMEIRNLGGDDTAVYYCARWSWSDGYYVYFDYWGQGTSVTVSS

SEQ ID NO: 29 - protein, 5 aa, Artificial Sequence
  CD33 HCDR1 E11
NYGMN

SEQ ID NO: 30 - protein, 17 aa, Artificial Sequence
  CD33 HCDR2 E11
WINTYTGEPTYADKFQG

SEQ ID NO: 31 - protein, 13 aa, Artificial Sequence
  CD33 HCDR3 E11
WSWSDGYYVYFDY

SEQ ID NO: 32 - protein, 113 aa, Artificial Sequence
  CD33 CC VL E11
DIVMTQSPDSLTVSLGERTTINCKSSQSVLDSSTNKNSLAWYQQKPGQPPKLLLSWASTRESGIPDRFSGSGSGTDFTLTIDSPQPEDSATYYCQQSAHFPITFGCGTRLEIK

SEQ ID NO: 33 - protein, 113 aa, Artificial Sequence
  CD33 VL E11
DIVMTQSPDSLTVSLGERTTINCKSSQSVLDSSTNKNSLAWYQQKPGQPPKLLLSWASTRESGIPDRFSGSGSGTDFTLTIDSPQPEDSATYYCQQSAHFPITFGQGTRLEIK

SEQ ID NO: 34 - protein, 17 aa, Artificial Sequence
  CD33 LCDR1 E11
KSSQSVLDSSTNKNSLA

SEQ ID NO: 35 - protein, 7 aa, Artificial Sequence
  CD33 LCDR2 E11
WASTRES

SEQ ID NO: 36 - protein, 9 aa, Artificial Sequence
  CD33 LCDR3 E11
QQSAHFPIT

SEQ ID NO: 37 - protein, 250 aa, Artificial Sequence
  CD33 HL CC E11
QVQLVQSGAEVKKPGESVKVSCKASGYTFTNYGMNWVKQAPGQCLEWMGWINTYTGEPTYADKFQGRVTMTTDTSTSTAYMEIRNLGGDDTAVYYCARWSWSDGYYVYFDYWGQGTSVTVSSGGGGSGGGGSGGGGSDIVMTQSPDSLTVSLGERTTINCKSSQSVLDSSTNKNSLAWYQQKPGQPPKLLLSWASTRESGIPDRFSGSGSGTDFTLTIDSPQPEDSATYYCQQSAHFPITFGCGTRLEIK

SEQ ID NO: 38 - protein, 250 aa, Artificial Sequence
  CD33 HL E11
QVQLVQSGAEVKKPGESVKVSCKASGYTFTNYGMNWVKQAPGQGLEWMGWINTYTGEPTYADKFQGRVTMTTDTSTSTAYMEIRNLGGDDTAVYYCARWSWSDGYYVYFDYWGQGTSVTVSSGGGGSGGGGSGGGGSDIVMTQSPDSLTVSLGERTTINCKSSQSVLDSSTNKNSLAWYQQKPGQPPKLLLSWASTRESGIPDRFSGSGSGTDFTLTIDSPQPEDSATYYCQQSAHFPITFGQGTRLEIK

SEQ ID NO: 39 - protein, 505 aa, Artificial Sequence
  CD33 CC E11 HL x I2C HL Bispecific molecule
QVQLVQSGAEVKKPGESVKVSCKASGYTFTNYGMNWVKQAPGQCLEWMGWINTYTGEPTYADKFQGRVTMTTDTSTSTAYMEIRNLGGDDTAVYYCARWSWSDGYYVYFDYWGQGTSVTVSSGGGGSGGGGSGGGGSDIVMTQSPDSLTVSLGERTTINCKSSQSVLDSSTNKNSLAWYQQKPGQPPKLLLSWASTRESGIPDRFSGSGSGTDFTLTIDSPQPEDSATYYCQQSAHFPITFGCGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 40 - protein, 530 aa, Artificial Sequence
  CD33 E11 HL x I2C HL
MGWSCIILFLVATATGVHSQVQLVQSGAEVKKPGESVKVSCKASGYTFTNYGMNWVKQAPGQGLEWMGWINTYTGEPTYADKFQGRVTMTTDTSTSTAYMEIRNLGGDDTAVYYCARWSWSDGYYVYFDYWGQGTSVTVSSGGGGSGGGGSGGGGSDIVMTQSPDSLTVSLGERTTINCKSSQSVLDSSTNKNSLAWYQQKPGQPPKLLLSWASTRESGIPDRFSGSGSGTDFTLTIDSPQPEDSATYYCQQSAHFPITFGQGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLHHHHHH

SEQ ID NO: 41 - protein, 993 aa, Artificial Sequence
  CD33 CC x I2C-scFc Bispecific HLE molecule
QVQLVQSGAEVKKPGESVKVSCKASGYTFTNYGMNWVKQAPGQCLEWMGWINTYTGEPTYADKFQGRVTMTTDTSTSTAYMEIRNLGGDDTAVYYCARWSWSDGYYVYFDYWGQGTSVTVSSGGGGSGGGGSGGGGSDIVMTQSPDSLTVSLGERTTINCKSSQSVLDSSTNKNSLAWYQQKPGQPPKLLLSWASTRESGIPDRFSGSGSGTDFTLTIDSPQPEDSATYYCQQSAHFPITFGCGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 42 - protein, 5 aa, Artificial Sequence
  EGFRvIIIxCD3-scFc VH CDR1
NYGMH

SEQ ID NO: 43 - protein, 17 aa, Artificial Sequence
  EGFRvIIIxCD3-scFc VH CDR2
VIWYDGSDKYYADSVRG

SEQ ID NO: 44 - protein, 15 aa, Artificial Sequence
  EGFRvIIIxCD3-scFc VH CDR3
DGYDILTGNPRDFDY

SEQ ID NO: 45 - protein, 16 aa, Artificial Sequence
  EGFRvIIIxCD3-scFc VL CDR1
RSSQSLVHSDGNTYLS

SEQ ID NO: 46 - protein, 7 aa, Artificial Sequence
  EGFRvIIIxCD3-scFc VL CDR2
RISRRFS

SEQ ID NO: 47 - protein, 9 aa, Artificial Sequence
  EGFRvIIIxCD3-scFc VL CDR3
MQSTHVPRT

SEQ ID NO: 48 - protein, 124 aa, Artificial Sequence
  EGFRvIII_CCxCD3-scFc VH
QVQLVESGGGVVQSGRSLRLSCAASGFTFRNYGMHWVRQAPGKCLEWVAVIWYDGSDKYYADSVRGRFTISRDNSKNTLYLQMNSLRAEDTAVYYCARDGYDILTGNPRDFDYWGQGTLVTVSS

SEQ ID NO: 49 - protein, 112 aa, Artificial Sequence
  EGFRvIII_CCxCD3-scFc VL
DTVMTQTPLSSHVTLGQPASISCRSSQSLVHSDGNTYLSWLQQRPGQPPRLLIYRISRRFSGVPDRFSGSGAGTDFTLEISRVEAEDVGVYYCMQSTHVPRTFGCGTKVEIK

SEQ ID NO: 50 - protein, 251 aa, Artificial Sequence
  EGFRvIII_CCxCD3-scFc scFv
QVQLVESGGGVVQSGRSLRLSCAASGFTFRNYGMHWVRQAPGKCLEWVAVIWYDGSDKYYADSVRGRFTISRDNSKNTLYLQMNSLRAEDTAVYYCARDGYDILTGNPRDFDYWGQGTLVTVSSGGGGSGGGGSGGGGSDTVMTQTPLSSHVTLGQPASISCRSSQSLVHSDGNTYLSWLQQRPGQPPRLLIYRISRRFSGVPDRFSGSGAGTDFTLEISRVEAEDVGVYYCMQSTHVPRTFGCGTKVEIK

SEQ ID NO: 51 - protein, 506 aa, Artificial Sequence
  EGFRvIII_CCxCD3-scFc Bispecific molecule
QVQLVESGGGVVQSGRSLRLSCAASGFTFRNYGMHWVRQAPGKCLEWVAVIWYDGSDKYYADSVRGRFTISRDNSKNTLYLQMNSLRAEDTAVYYCARDGYDILTGNPRDFDYWGQGTLVTVSSGGGGSGGGGSGGGGSDTVMTQTPLSSHVTLGQPASISCRSSQSLVHSDGNTYLSWLQQRPGQPPRLLIYRISRRFSGVPDRFSGSGAGTDFTLEISRVEAEDVGVYYCMQSTHVPRTFGCGTKVEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 52 - protein, 994 aa, Artificial Sequence
  EGFRvIII_CCxCD3-scFc Bispecific HLE molecule
QVQLVESGGGVVQSGRSLRLSCAASGFTFRNYGMHWVRQAPGKCLEWVAVIWYDGSDKYYADSVRGRFTISRDNSKNTLYLQMNSLRAEDTAVYYCARDGYDILTGNPRDFDYWGQGTLVTVSSGGGGSGGGGSGGGGSDTVMTQTPLSSHVTLGQPASISCRSSQSLVHSDGNTYLSWLQQRPGQPPRLLIYRISRRFSGVPDRFSGSGAGTDFTLEISRVEAEDVGVYYCMQSTHVPRTFGCGTKVEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 53 - protein, 5 aa, Artificial Sequence
  MSLN_5 VH CDR1
DYYMT

SEQ ID NO: 54 - protein, 17 aa, Artificial Sequence
  MSLN_5 VH CDR2
YISSSGSTIYYADSVKG

SEQ ID NO: 55 - protein, 8 aa, Artificial Sequence
  MSLN_5 VH CDR3
DRNSHFDY

SEQ ID NO: 56 - protein, 11 aa, Artificial Sequence
  MSLN_5 VL CDR1
RASQGINTWLA

SEQ ID NO: 57 - protein, 7 aa, Artificial Sequence
  MSLN_5 VL CDR2
GASGLQS

SEQ ID NO: 58 - protein, 9 aa, Artificial Sequence
  MSLN_5 VL CDR3
QQAKSFPRT

SEQ ID NO: 59 - protein, 117 aa, Artificial Sequence
  MSLN_5 VH
QVQLVESGGGLVKPGGSLRLSCAASGFTFSDYYMTWIRQAPGKGLEWLSYISSSGSTIYYADSVKGRFTISRDNAKNSLFLQMNSLRAEDTAVYYCARDRNSHFDYWGQGTLVTVSS

SEQ ID NO: 60 - protein, 107 aa, Artificial Sequence
  MSLN_5 VL
DIQMTQSPSSVSASVGDRVTITCRASQGINTWLAWYQQKPGKAPKLLIYGASGLQSGVPSRFSGSGSGTDFTLTISSLQPEDFATYYCQQAKSFPRTFGQGTKVEIK

SEQ ID NO: 61 - protein, 239 aa, Artificial Sequence
  MSLN_5 scFv
QVQLVESGGGLVKPGGSLRLSCAASGFTFSDYYMTWIRQAPGKGLEWLSYISSSGSTIYYADSVKGRFTISRDNAKNSLFLQMNSLRAEDTAVYYCARDRNSHFDYWGQGTLVTVSSGGGGSGGGGSGGGGSDIQMTQSPSSVSASVGDRVTITCRASQGINTWLAWYQQKPGKAPKLLIYGASGLQSGVPSRFSGSGSGTDFTLTISSLQPEDFATYYCQQAKSFPRTFGQGTKVEIK

SEQ ID NO: 62 - protein, 494 aa, Artificial Sequence
  MSLN_5xI2C0 bispecific molecule
QVQLVESGGGLVKPGGSLRLSCAASGFTFSDYYMTWIRQAPGKGLEWLSYISSSGSTIYYADSVKGRFTISRDNAKNSLFLQMNSLRAEDTAVYYCARDRNSHFDYWGQGTLVTVSSGGGGSGGGGSGGGGSDIQMTQSPSSVSASVGDRVTITCRASQGINTWLAWYQQKPGKAPKLLIYGASGLQSGVPSRFSGSGSGTDFTLTISSLQPEDFATYYCQQAKSFPRTFGQGTKVEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 63 - protein, 982 aa, Artificial Sequence
  MSLN_5xCD3-scFc Bispecific HLE molecule
QVQLVESGGGLVKPGGSLRLSCAASGFTFSDYYMTWIRQAPGKGLEWLSYISSSGSTIYYADSVKGRFTISRDNAKNSLFLQMNSLRAEDTAVYYCARDRNSHFDYWGQGTLVTVSSGGGGSGGGGSGGGGSDIQMTQSPSSVSASVGDRVTITCRASQGINTWLAWYQQKPGKAPKLLIYGASGLQSGVPSRFSGSGSGTDFTLTISSLQPEDFATYYCQQAKSFPRTFGQGTKVEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 64 - protein, 982 aa, Artificial Sequence
  MSLN_5_CCxCD3-scFc Bispecific HLE molecule
QVQLVESGGGLVKPGGSLRLSCAASGFTFSDHYMSWIRQAPGKCLEWFSYISSSGGIIYYADSVKGRFTISRDNAKNSLYLQMNSLRAEDTAVYYCARDVGSHFDYWGQGTLVTVSSGGGGSGGGGSGGGGSDIQMTQSPSSVSASVGDRVTITCRASQDISRWLAWYQQKPGKAPKLLISAASRLQSGVPSRFSGSGSGTDFTLTISSLQPEDFAIYYCQQAKSFPRTFGCGTKVEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 65 - protein, 5 aa, Artificial Sequence
  CDR-H1 CDH19 65254.007
SYGMH

SEQ ID NO: 66 - protein, 17 aa, Artificial Sequence
  CDR-H2 CDH19 65254.007
FIWYEGSNKYYAESVKD

SEQ ID NO: 67 - protein, 16 aa, Artificial Sequence
  CDR-H3 CDH19 65254.007
RAGIIGTIGYYYGMDV

SEQ ID NO: 68 - protein, 11 aa, Artificial Sequence
  CDR-L1 CDH19 65254.007
SGDRLGEKYTS

SEQ ID NO: 69 - protein, 7 aa, Artificial Sequence
  CDR-L2 CDH19 65254.007
QDTKRPS

SEQ ID NO: 70 - protein, 9 aa, Artificial Sequence
  CDR-L3 CDH19 65254.007
QAWESSTVV

SEQ ID NO: 71 - protein, 125 aa, Artificial Sequence
  VH CDH19 65254.007
QVQLVESGGGVVQPGGSLRLSCAASGFTFSSYGMHWVRQAPGKGLEWVAFIWYEGSNKYYAESVKDRFTISRDNSKNTLYLQMNSLRAEDTAVYYCARRAGIIGTIGYYYGMDVWGQGTTVTVSS

SEQ ID NO: 72 - protein, 107 aa, Artificial Sequence
  VL CDH19 65254.007
SYELTQPPSVSVSPGQTASITCSGDRLGEKYTSWYQQRPGQSPLLVIYQDTKRPSGIPERFSGSNSGNTATLTISGTQAMDEADYYCQAWESSTVVFGGGTKLTVLS

SEQ ID NO: 73 - protein, 247 aa, Artificial Sequence
  VH-VL CDH19 65254.007
QVQLVESGGGVVQPGGSLRLSCAASGFTFSSYGMHWVRQAPGKGLEWVAFIWYEGSNKYYAESVKDRFTISRDNSKNTLYLQMNSLRAEDTAVYYCARRAGIIGTIGYYYGMDVWGQGTTVTVSSGGGGSGGGGSGGGGSSYELTQPPSVSVSPGQTASITCSGDRLGEKYTSWYQQRPGQSPLLVIYQDTKRPSGIPERFSGSNSGNTATLTISGTQAMDEADYYCQAWESSTVVFGGGTKLTVLS

SEQ ID NO: 74 - protein, 507 aa, Artificial Sequence
  CDH19 65254.007 x I2C
QVQLVESGGGVVQPGGSLRLSCAASGFTFSSYGMHWVRQAPGKGLEWVAFIWYEGSNKYYAESVKDRFTISRDNSKNTLYLQMNSLRAEDTAVYYCARRAGIIGTIGYYYGMDVWGQGTTVTVSSGGGGSGGGGSGGGGSSYELTQPPSVSVSPGQTASITCSGDRLGEKYTSWYQQRPGQSPLLVIYQDTKRPSGIPERFSGSNSGNTATLTISGTQAMDEADYYCQAWESSTVVFGGGTKLTVLSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLHHHHHH

SEQ ID NO: 75 - protein, 989 aa, Artificial Sequence
  CDH19 65254.007 x I2C -scFc Bispecific HLE molecule
QVQLVESGGGVVQPGGSLRLSCAASGFTFSSYGMHWVRQAPGKGLEWVAFIWYEGSNKYYAESVKDRFTISRDNSKNTLYLQMNSLRAEDTAVYYCARRAGIIGTIGYYYGMDVWGQGTTVTVSSGGGGSGGGGSGGGGSSYELTQPPSVSVSPGQTASITCSGDRLGEKYTSWYQQRPGQSPLLVIYQDTKRPSGIPERFSGSNSGNTATLTISGTQAMDEADYYCQAWESSTVVFGGGTKLTVLSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 76 - protein, 987 aa, Artificial Sequence
  CDH19 65254.007 x I2C -scFc_delGK Bispecific HLE molecule
QVQLVESGGGVVQPGGSLRLSCAASGFTFSSYGMHWVRQAPGKGLEWVAFIWYEGSNKYYAESVKDRFTISRDNSKNTLYLQMNSLRAEDTAVYYCARRAGIIGTIGYYYGMDVWGQGTTVTVSSGGGGSGGGGSGGGGSSYELTQPPSVSVSPGQTASITCSGDRLGEKYTSWYQQRPGQSPLLVIYQDTKRPSGIPERFSGSNSGNTATLTISGTQAMDEADYYCQAWESSTVVFGGGTKLTVLSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 77 - protein, 125 aa, Artificial Sequence
  CDH19 65254.007_CC x I2C -scFc VH
QVQLVESGGGVVQPGGSLRLSCAASGFTFSSYGMHWVRQAPGKCLEWVAFIWYEGSNKYYAESVKDRFTISRDNSKNTLYLQMNSLRAEDTAVYYCARRAGIIGTIGYYYGMDVWGQGTTVTVSS

SEQ ID NO: 78 - protein, 106 aa, Artificial Sequence
  CDH19 65254.007_CC x I2C -scFc VL
SYELTQPPSVSVSPGQTASITCSGDRLGEKYTSWYQQRPGQSPLLVIYQDTKRPSGIPERFSGSNSGNTATLTISGTQAMDEADYYCQAWESSTVVFGCGTKLTVL

SEQ ID NO: 79 - protein, 246 aa, Artificial Sequence
  CDH19 65254.007_CC x I2C -scFc scFv
QVQLVESGGGVVQPGGSLRLSCAASGFTFSSYGMHWVRQAPGKCLEWVAFIWYEGSNKYYAESVKDRFTISRDNSKNTLYLQMNSLRAEDTAVYYCARRAGIIGTIGYYYGMDVWGQGTTVTVSSGGGGSGGGGSGGGGSSYELTQPPSVSVSPGQTASITCSGDRLGEKYTSWYQQRPGQSPLLVIYQDTKRPSGIPERFSGSNSGNTATLTISGTQAMDEADYYCQAWESSTVVFGCGTKLTVL

SEQ ID NO: 80 - protein, 501 aa, Artificial Sequence
  CDH19 65254.007_CC x I2C -scFc Bispecific molecule
QVQLVESGGGVVQPGGSLRLSCAASGFTFSSYGMHWVRQAPGKCLEWVAFIWYEGSNKYYAESVKDRFTISRDNSKNTLYLQMNSLRAEDTAVYYCARRAGIIGTIGYYYGMDVWGQGTTVTVSSGGGGSGGGGSGGGGSSYELTQPPSVSVSPGQTASITCSGDRLGEKYTSWYQQRPGQSPLLVIYQDTKRPSGIPERFSGSNSGNTATLTISGTQAMDEADYYCQAWESSTVVFGCGTKLTVLSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 81 - protein, 989 aa, Artificial Sequence
  CDH19 65254.007_CC x I2C -scFc Bispecific HLE molecule
QVQLVESGGGVVQPGGSLRLSCAASGFTFSSYGMHWVRQAPGKCLEWVAFIWYEGSNKYYAESVKDRFTISRDNSKNTLYLQMNSLRAEDTAVYYCARRAGIIGTIGYYYGMDVWGQGTTVTVSSGGGGSGGGGSGGGGSSYELTQPPSVSVSPGQTASITCSGDRLGEKYTSWYQQRPGQSPLLVIYQDTKRPSGIPERFSGSNSGNTATLTISGTQAMDEADYYCQAWESSTVVFGCGTKLTVLSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 82 - protein, 987 aa, Artificial Sequence
  CDH19 65254.007_CC x I2C -scFc_delGK Bispecific HLE molecule
QVQLVESGGGVVQPGGSLRLSCAASGFTFSSYGMHWVRQAPGKCLEWVAFIWYEGSNKYYAESVKDRFTISRDNSKNTLYLQMNSLRAEDTAVYYCARRAGIIGTIGYYYGMDVWGQGTTVTVSSGGGGSGGGGSGGGGSSYELTQPPSVSVSPGQTASITCSGDRLGEKYTSWYQQRPGQSPLLVIYQDTKRPSGIPERFSGSNSGNTATLTISGTQAMDEADYYCQAWESSTVVFGCGTKLTVLSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 83 - protein, 7 aa, Artificial Sequence
  FLT3_7 A8xCD3-scFc VH CDR1
NARMGVS

SEQ ID NO: 84 - protein, 16 aa, Artificial Sequence
  FLT3_7 A8xCD3-scFc VH CDR2
HIFSNDEKSYSTSLKN

SEQ ID NO: 85 - protein, 14 aa, Artificial Sequence
  FLT3_7 A8xCD3-scFc VH CDR3
IVGYGSGWYGFFDY

SEQ ID NO: 86 - protein, 11 aa, Artificial Sequence
  FLT3_7 A8xCD3-scFc VL CDR1
RASQGIRNDLG

SEQ ID NO: 87 - protein, 7 aa, Artificial Sequence
  FLT3_7 A8xCD3-scFc VL CDR2
AASTLQS

SEQ ID NO: 88 - protein, 9 aa, Artificial Sequence
  FLT3_7 A8xCD3-scFc VL CDR3
LQHNSYPLT

SEQ ID NO: 89 - protein, 124 aa, Artificial Sequence
  FLT3_7 A8xCD3-scFc VH
QVTLKESGPTLVKPTETLTLTCTLSGFSLNNARMGVSWIRQPPGKCLEWLAHIFSNDEKSYSTSLKNRLTISKDSSKTQVVLTMTNVDPVDTATYYCARIVGYGSGWYGFFDYWGQGTLVTVSS

SEQ ID NO: 90 - protein, 107 aa, Artificial Sequence
  FLT3_ A8-scFc VL
DIQMTQSPSSLSASVGDRVTITCRASQGIRNDLGWYQQKPGKAPKRLIYAASTLQSGVPSRFSGSGSGTEFTLTISSLQPEDFATYYCLQHNSYPLTFGCGTKVEIK

SEQ ID NO: 91 - protein, 246 aa, Artificial Sequence
  FLT3_7 A8xCD3- scFv
QVTLKESGPTLVKPTETLTLTCTLSGFSLNNARMGVSWIRQPPGKCLEWLAHIFSNDEKSYSTSLKNRLTISKDSSKTQVVLTMTNVDPVDTATYYCARIVGYGSGWYGFFDYWGQGTLVTVSSGGGGSGGGGSGGGGSDIQMTQSPSSLSASVGDRVTITCRASQGIRNDLGWYQQKPGKAPKRLIYAASTLQSGVPSRFSGSGSGTEFTLTISSLQPEDFATYYCLQHNSYPLTFGCGTKVEIK

SEQ ID NO: 92 - protein, 501 aa, Artificial Sequence
  FLT3_7 A8xCD3 Bispecific molecule
QVTLKESGPTLVKPTETLTLTCTLSGFSLNNARMGVSWIRQPPGKCLEWLAHIFSNDEKSYSTSLKNRLTISKDSSKTQVVLTMTNVDPVDTATYYCARIVGYGSGWYGFFDYWGQGTLVTVSSGGGGSGGGGSGGGGSDIQMTQSPSSLSASVGDRVTITCRASQGIRNDLGWYQQKPGKAPKRLIYAASTLQSGVPSRFSGSGSGTEFTLTISSLQPEDFATYYCLQHNSYPLTFGCGTKVEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 93 - protein, 989 aa, Artificial Sequence
  FLT3_7 A8xCD3-scFc Bispecific HLE molecule
QVTLKESGPTLVKPTETLTLTCTLSGFSLNNARMGVSWIRQPPGKCLEWLAHIFSNDEKSYSTSLKNRLTISKDSSKTQVVLTMTNVDPVDTATYYCARIVGYGSGWYGFFDYWGQGTLVTVSSGGGGSGGGGSGGGGSDIQMTQSPSSLSASVGDRVTITCRASQGIRNDLGWYQQKPGKAPKRLIYAASTLQSGVPSRFSGSGSGTEFTLTISSLQPEDFATYYCLQHNSYPLTFGCGTKVEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 94 - protein, 5 aa, Artificial Sequence
  VH CDR1 DLL3_1_CC_delGK
SYYWS

SEQ ID NO: 95 - protein, 16 aa, Artificial Sequence
  VH CDR2 DLL3_1_CC_delGK
YVYYSGTTNYNPSLKS

SEQ ID NO: 96 - protein, 10 aa, Artificial Sequence
  VH CDR3 DLL3_1_CC_delGK
IAVTGFYFDY

SEQ ID NO: 97 - protein, 12 aa, Artificial Sequence
  VL CDR1 DLL3_1_CC_delGK
RASQRVNNNYLA

SEQ ID NO: 98 - protein, 7 aa, Artificial Sequence
  VL CDR2 DLL3_1_CC_delGK
GASSRAT

SEQ ID NO: 99 - protein, 9 aa, Artificial Sequence
  VL CDR3 DLL3_1_CC_delGK
QQYDRSPLT

SEQ ID NO: 100 - protein, 118 aa, Artificial Sequence
  VH DLL3_1_CC_delGK
QVQLQESGPGLVKPSETLSLTCTVSGGSISSYYWSWIRQPPGKCLEWIGYVYYSGTTNYNPSLKSRVTISVDTSKNQFSLKLSSVTAADTAVYYCASIAVTGFYFDYWGQGTLVTVSS

SEQ ID NO: 101 - protein, 108 aa, Artificial Sequence
  VL DLL3_1_CC_delGK
EIVLTQSPGTLSLSPGERVTLSCRASQRVNNNYLAWYQQRPGQAPRLLIYGASSRATGIPDRFSGSGSGTDFTLTISRLEPEDFAVYYCQQYDRSPLTFGCGTKLEIK

SEQ ID NO: 102 - protein, 241 aa, Artificial Sequence
  DLL3_1_CC_delGK
QVQLQESGPGLVKPSETLSLTCTVSGGSISSYYWSWIRQPPGKCLEWIGYVYYSGTTNYNPSLKSRVTISVDTSKNQFSLKLSSVTAADTAVYYCASIAVTGFYFDYWGQGTLVTVSSGGGGSGGGGSGGGGSEIVLTQSPGTLSLSPGERVTLSCRASQRVNNNYLAWYQQRPGQAPRLLIYGASSRATGIPDRFSGSGSGTDFTLTISRLEPEDFAVYYCQQYDRSPLTFGCGTKLEIK

SEQ ID NO: 103 - protein, 496 aa, Artificial Sequence
  DLL3_1_CCxCD3_delGK Bispecific molecule
QVQLQESGPGLVKPSETLSLTCTVSGGSISSYYWSWIRQPPGKCLEWIGYVYYSGTTNYNPSLKSRVTISVDTSKNQFSLKLSSVTAADTAVYYCASIAVTGFYFDYWGQGTLVTVSSGGGGSGGGGSGGGGSEIVLTQSPGTLSLSPGERVTLSCRASQRVNNNYLAWYQQRPGQAPRLLIYGASSRATGIPDRFSGSGSGTDFTLTISRLEPEDFAVYYCQQYDRSPLTFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 104 - protein, 982 aa, Artificial Sequence
  DLL3_1_CCxCD3-scFc_delGK Bispecific HLE molecule
QVQLQESGPGLVKPSETLSLTCTVSGGSISSYYWSWIRQPPGKCLEWIGYVYYSGTTNYNPSLKSRVTISVDTSKNQFSLKLSSVTAADTAVYYCASIAVTGFYFDYWGQGTLVTVSSGGGGSGGGGSGGGGSEIVLTQSPGTLSLSPGERVTLSCRASQRVNNNYLAWYQQRPGQAPRLLIYGASSRATGIPDRFSGSGSGTDFTLTISRLEPEDFAVYYCQQYDRSPLTFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 105 - protein, 5 aa, Artificial Sequence
  VH CDR1 CD19 97-G1RE-C2
SYGMH

SEQ ID NO: 106 - protein, 17 aa, Artificial Sequence
  VH CDR2 CD19 97-G1RE-C2
VISYEGSNKYYAESVKG

SEQ ID NO: 107 - protein, 13 aa, Artificial Sequence
  VH CDR3 CD19 97-G1RE-C2
DRGTIFGNYGLEV

SEQ ID NO: 108 - protein, 122 aa, Artificial Sequence
  VH CD19 97-G1RE-C2 CC
QVQLVESGGGVVQPGRSLRLSCAASGFTFSSYGMHWVRQAPGKCLEWVAVISYEGSNKYYAESVKGRFTISRDNSKNTLYLQMNSLRDEDTAVYYCARDRGTIFGNYGLEVWGQGTTVTVSS

SEQ ID NO: 109 - protein, 16 aa, Artificial Sequence
  VL CDR1 CD19 97-G1RE-C2
RSSQSLLHKNAFNYLD

SEQ ID NO: 110 - protein, 7 aa, Artificial Sequence
  VL CDR2 CD19 97-G1RE-C2
LGSNRAS

SEQ ID NO: 111 - protein, 9 aa, Artificial Sequence
  VL CDR3 CD19 97-G1RE-C2
MQALQTPFT

SEQ ID NO: 112 - protein, 112 aa, Artificial Sequence
  VL CD19 97-G1RE-C2 CC
DIVMTQSPLSLPVISGEPASISCRSSQSLLHKNAFNYLDWYLQKPGQSPQLLIYLGSNRASGVPDRFSGSGSGTDFTLKISRVEAEDVGVYYCMQALQTPFTFGCGTKVDIK

SEQ ID NO: 113 - protein, 525 aa, Artificial Sequence
  CD19 97-G1RE-C2 CC x I2C0
MDMRVPAQLLGLLLLWLRGARCDIVMTQSPLSLPVISGEPASISCRSSQSLLHKNAFNYLDWYLQKPGQSPQLLIYLGSNRASGVPDRFSGSGSGTDFTLKISRVEAEDVGVYYCMQALQTPFTFGCGTKVDIKGGGGSGGGGSGGGGSQVQLVESGGGVVQPGRSLRLSCAASGFTFSSYGMHWVRQAPGKCLEWVAVISYEGSNKYYAESVKGRFTISRDNSKNTLYLQMNSLRDEDTAVYYCARDRGTIFGNYGLEVWGQGTTVTVSSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 114 - protein, 1013 aa, Artificial Sequence
  CD19 97-G1RE-C2 CC x I2C0-scFc
MDMRVPAQLLGLLLLWLRGARCDIVMTQSPLSLPVISGEPASISCRSSQSLLHKNAFNYLDWYLQKPGQSPQLLIYLGSNRASGVPDRFSGSGSGTDFTLKISRVEAEDVGVYYCMQALQTPFTFGCGTKVDIKGGGGSGGGGSGGGGSQVQLVESGGGVVQPGRSLRLSCAASGFTFSSYGMHWVRQAPGKCLEWVAVISYEGSNKYYAESVKGRFTISRDNSKNTLYLQMNSLRDEDTAVYYCARDRGTIFGNYGLEVWGQGTTVTVSSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 115 - protein, 5 aa, Artificial Sequence
  VH CDR1 CDH3 G8A 6-B12
SYPIN

SEQ ID NO: 116 - protein, 16 aa, Artificial Sequence
  VH CDR2 CDH3 G8A 6-B12
VIWTGGGTNYASSVKG

SEQ ID NO: 117 - protein, 15 aa, Artificial Sequence
  VH CDR3 CDH3 G8A 6-B12
SRGVYDFDGRGAMDY

SEQ ID NO: 118 - protein, 17 aa, Artificial Sequence
  VL CDR1 CDH3 G8A 6-B12
KSSQSLLYSSNQKNYFA

SEQ ID NO: 119 - protein, 7 aa, Artificial Sequence
  VL CDR2 CDH3 G8A 6-B12
WASTRES

SEQ ID NO: 120 - protein, 9 aa, Artificial Sequence
  VL CDR3 CDH3 G8A 6-B12
QQYYSYPYT

SEQ ID NO: 121 - protein, 123 aa, Artificial Sequence
  VH CDH3 G8A 6-B12
EVQLLESGGGLVQPGGSLRLSCAASGFSFSSYPINWVRQAPGKGLEWVGVIWTGGGTNYASSVKGRFTISRDNSKNTVYLQMNSLRAEDTAVYYCAKSRGVYDFDGRGAMDYWGQGTLVTVSS

SEQ ID NO: 122 - protein, 113 aa, Artificial Sequence
  VL CDH3 G8A 6-B12
DIVMTQSPDSLAVSLGERATINCKSSQSLLYSSNQKNYFAWYQQKPGQPPKLLIYWASTRESGVPDRFSGSGSGTDFTLTISSLQAEDVAVYYCQQYYSYPYTFGQGTKLEIK

SEQ ID NO: 123 - protein, 251 aa, Artificial Sequence
  CDH3 G8A 6-B12 scFv
EVQLLESGGGLVQPGGSLRLSCAASGFSFSSYPINWVRQAPGKGLEWVGVIWTGGGTNYASSVKGRFTISRDNSKNTVYLQMNSLRAEDTAVYYCAKSRGVYDFDGRGAMDYWGQGTLVTVSSGGGGSGGGGSGGGGSDIVMTQSPDSLAVSLGERATINCKSSQSLLYSSNQKNYFAWYQQKPGQPPKLLIYWASTRESGVPDRFSGSGSGTDFTLTISSLQAEDVAVYYCQQYYSYPYTFGQGTKLEIK

SEQ ID NO: 124 - protein, 506 aa, Artificial Sequence
  CDH3 G8A 6-B12 x I2C0 bispecific molecule
EVQLLESGGGLVQPGGSLRLSCAASGFSFSSYPINWVRQAPGKGLEWVGVIWTGGGTNYASSVKGRFTISRDNSKNTVYLQMNSLRAEDTAVYYCAKSRGVYDFDGRGAMDYWGQGTLVTVSSGGGGSGGGGSGGGGSDIVMTQSPDSLAVSLGERATINCKSSQSLLYSSNQKNYFAWYQQKPGQPPKLLIYWASTRESGVPDRFSGSGSGTDFTLTISSLQAEDVAVYYCQQYYSYPYTFGQGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 125 - protein, 994 aa, Artificial Sequence
  CDH3 G8A 6-B12 x I2C0 bispecific molecule HLE
EVQLLESGGGLVQPGGSLRLSCAASGFSFSSYPINWVRQAPGKGLEWVGVIWTGGGTNYASSVKGRFTISRDNSKNTVYLQMNSLRAEDTAVYYCAKSRGVYDFDGRGAMDYWGQGTLVTVSSGGGGSGGGGSGGGGSDIVMTQSPDSLAVSLGERATINCKSSQSLLYSSNQKNYFAWYQQKPGQPPKLLIYWASTRESGVPDRFSGSGSGTDFTLTISSLQAEDVAVYYCQQYYSYPYTFGQGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 126 - protein, 5 aa, Artificial Sequence
  BCMA A7 27-C4-G7 CDR1 VH
NHIIH

SEQ ID NO: 127 - protein, 17 aa, Artificial Sequence
  BCMA A7 27-C4-G7 CDR2 VH
YINPYPGYHAYNEKFQG

SEQ ID NO: 128 - protein, 12 aa, Artificial Sequence
  BCMA A7 27-C4-G7 CDR3 VH
DGYYRDTDVLDY

SEQ ID NO: 129 - protein, 11 aa, Artificial Sequence
  BCMA A7 27-C4-G7 CDR1 VL
QASQDISNYLN

SEQ ID NO: 130 - protein, 7 aa, Artificial Sequence
  BCMA A7 27-C4-G7 CDR2 VL
YTSRLHT

SEQ ID NO: 131 - protein, 9 aa, Artificial Sequence
  BCMA A7 27-C4-G7 CDR3 VL
QQGNTLPWT

SEQ ID NO: 132 - protein, 121 aa, Artificial Sequence
  BCMA A7 27-C4-G7 CC (44/100) VH
QVQLVQSGAEVKKPGASVKVSCKASGYTFTNHIIHWVRQAPGQCLEWMGYINPYPGYHAYNEKFQGRATMTSDTSTSTVYMELSSLRSEDTAVYYCARDGYYRDTDVLDYWGQGTLVTVSS

SEQ ID NO: 133 - protein, 107 aa, Artificial Sequence
  BCMA A7 27-C4-G7 CC (44/100) VL
DIQMTQSPSSLSASVGDRVTITCQASQDISNYLNWYQQKPGKAPKLLIYYTSRLHTGVPSRFSGSGSGTDFTFTISSLEPEDIATYYCQQGNTLPWTFGCGTKLEIK

SEQ ID NO: 134 - protein, 243 aa, Artificial Sequence
  BCMA A7 27-C4-G7 CC (44/100) scFv
QVQLVQSGAEVKKPGASVKVSCKASGYTFTNHIIHWVRQAPGQCLEWMGYINPYPGYHAYNEKFQGRATMTSDTSTSTVYMELSSLRSEDTAVYYCARDGYYRDTDVLDYWGQGTLVTVSSGGGGSGGGGSGGGGSDIQMTQSPSSLSASVGDRVTITCQASQDISNYLNWYQQKPGKAPKLLIYYTSRLHTGVPSRFSGSGSGTDFTFTISSLEPEDIATYYCQQGNTLPWTFGCGTKLEIK

SEQ ID NO: 135 - protein, 498 aa, Artificial Sequence
  BCMA A7 27-C4-G7 CC (44/100) x I2C0 bispecific molecule
QVQLVQSGAEVKKPGASVKVSCKASGYTFTNHIIHWVRQAPGQCLEWMGYINPYPGYHAYNEKFQGRATMTSDTSTSTVYMELSSLRSEDTAVYYCARDGYYRDTDVLDYWGQGTLVTVSSGGGGSGGGGSGGGGSDIQMTQSPSSLSASVGDRVTITCQASQDISNYLNWYQQKPGKAPKLLIYYTSRLHTGVPSRFSGSGSGTDFTFTISSLEPEDIATYYCQQGNTLPWTFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 136 - protein, 986 aa, Artificial Sequence
  BCMA A7 27-C4-G7 CC (44/100) x I2C0-scFc bispecific molecule HLE
QVQLVQSGAEVKKPGASVKVSCKASGYTFTNHIIHWVRQAPGQCLEWMGYINPYPGYHAYNEKFQGRATMTSDTSTSTVYMELSSLRSEDTAVYYCARDGYYRDTDVLDYWGQGTLVTVSSGGGGSGGGGSGGGGSDIQMTQSPSSLSASVGDRVTITCQASQDISNYLNWYQQKPGKAPKLLIYYTSRLHTGVPSRFSGSGSGTDFTFTISSLEPEDIATYYCQQGNTLPWTFGCGTKVEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 137 - protein, 5 aa, Artificial Sequence
  PM 76-B10.17 CC VH CDR1
DYYMY

SEQ ID NO: 138 - protein, 17 aa, Artificial Sequence
  PM 76-B10.17 CC VH CDR2
IISDAGYYTYYSDIIKG

SEQ ID NO: 139 - protein, 12 aa, Artificial Sequence
  PM 76-B10.17 CC VH CDR3
GFPLLRHGAMDY

SEQ ID NO: 140 - protein, 11 aa, Artificial Sequence
  PM 76-B10.17 CC VL CDR1
KASQNVDANVA

SEQ ID NO: 141 - protein, 7 aa, Artificial Sequence
  PM 76-B10.17 CC VL CDR2
SASYVYW

SEQ ID NO: 142 - protein, 9 aa, Artificial Sequence
  PM 76-B10.17 CC VL CDR3
QQYDQQLIT

SEQ ID NO: 143 - protein, 121 aa, Artificial Sequence
  PM 76-B10.17 CC VH
QVQLVESGGGLVKPGESLRLSCAASGFTFSDYYMYWVRQAPGKCLEWVAIISDAGYYTYYSDIIKGRFTISRDNAKNSLYLQMNSLKAEDTAVYYCARGFPLLRHGAMDYWGQGTLVTVSS

SEQ ID NO: 144 - protein, 107 aa, Artificial Sequence
  PM 76-B10.17 CC VL
DIQMTQSPSSLSASVGDRVTITCKASQNVDANVAWYQQKPGQAPKSLIYSASYVYWDVPSRFSGSASGTDFTLTISSVQSEDFATYYCQQYDQQLITFGCGTKLEIK

SEQ ID NO: 145 - protein, 243 aa, Artificial Sequence
  PM 76-B10.17 CC scFv
QVQLVESGGGLVKPGESLRLSCAASGFTFSDYYMYWVRQAPGKCLEWVAIISDAGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDANVAWYQQKPGQAPKSLIYSASYVYWDVPSRFSGSASGTDFTLTISSVQSEDFATYYCQQYDQQLITFGCGTKLEIK

SEQ ID NO: 146 - protein, 498 aa, Artificial Sequence
  PM 76-B10.17 CC x I2C0 bispecific molecule
QVQLVESGGGLVKPGESLRLSCAASGFTFSDYYMYWVRQAPGKCLEWVAIISDAGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDANVAWYQQKPGQAPKSLIYSASYVYWDVPSRFSGSASGTDFTLTISSVQSEDFATYYCQQYDQQLITFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 147 - protein, 986 aa, Artificial Sequence
  PM 76-B10.17 CC x I2C0-scFc bispecific HLE molecule
QVQLVESGGGLVKPGESLRLSCAASGFTFSDYYMYWVRQAPGKCLEWVAIISDAGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDANVAWYQQKPGQAPKSLIYSASYVYWDVPSRFSGSASGTDFTLTISSVQSEDFATYYCQQYDQQLITFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 148 - protein, 984 aa, Artificial Sequence
  PM 76-B10.17 CC x I2C0-scFc_delGK bispecific HLE molecule
QVQLVESGGGLVKPGESLRLSCAASGFTFSDYYMYWVRQAPGKCLEWVAIISDAGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDANVAWYQQKPGQAPKSLIYSASYVYWDVPSRFSGSASGTDFTLTISSVQSEDFATYYCQQYDQQLITFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 149 - protein, 498 aa, Artificial Sequence
  PM 76-B10.17 CC x I2C0 CC (103/43)-scFc bispecific molecule
QVQLVESGGGLVKPGESLRLSCAASGFTFSDYYMYWVRQAPGKCLEWVAIISDAGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDANVAWYQQKPGQAPKSLIYSASYVYWDVPSRFSGSASGTDFTLTISSVQSEDFATYYCQQYDQQLITFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYCGQGTLVTVSSGGGGSGGGGSGGGGSQTVVTQEPSLTVSPGGTVTLTCGSSTGAVTSGNYPNWVQQKPGQCPRGLIGGTKFLAPGTPARFSGSLLGGKAALTLSGVQPEDEAEYYCVLWYSNRWVFGGGTKLTVL

SEQ ID NO: 150 - protein, 986 aa, Artificial Sequence
  PM 76-B10.17 CC x I2C0 CC (103/43)-scFc bispecific HLE molecule
QVQLVESGGGLVKPGESLRLSCAASGFTFSDYYMYWVRQAPGKCLEWVAIISDAGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDANVAWYQQKPGQAPKSLIYSASYVYWDVPSRFSGSASGTDFTLTISSVQSEDFATYYCQQYDQQLITFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYCGQGTLVTVSSGGGGSGGGGSGGGGSQTVVTQEPSLTVSPGGTVTLTCGSSTGAVTSGNYPNWVQQKPGQCPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 151 - protein, 984 aa, Artificial Sequence
  PM 76-B10.17 CC x I2C0 CC (103/43)-scFc delGK bispecific HLE molecule
QVQLVESGGGLVKPGESLRLSCAASGFTFSDYYMYWVRQAPGKCLEWVAIISDAGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDANVAWYQQKPGQAPKSLIYSASYVYWDVPSRFSGSASGTDFTLTISSVQSEDFATYYCQQYDQQLITFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYCGQGTLVTVSSGGGGSGGGGSGGGGSQTVVTQEPSLTVSPGGTVTLTCGSSTGAVTSGNYPNWVQQKPGQCPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 152 - protein, 5 aa, Artificial Sequence
  PM 76-B10.11 CC VH CDR1
DYYMY

SEQ ID NO: 153 - protein, 17 aa, Artificial Sequence
  PM 76-B10.11 CC VH CDR2
IISDGGYYTYYSDIIKG

SEQ ID NO: 154 - protein, 12 aa, Artificial Sequence
  PM 76-B10.11 CC VH CDR3
GFPLLRHGAMDY

SEQ ID NO: 155 - protein, 11 aa, Artificial Sequence
  PM 76-B10.11 CC VL CDR1
KASQNVDTNVA

SEQ ID NO: 156 - protein, 7 aa, Artificial Sequence
  PM 76-B10.11 CC VL CDR2
SASYVYW

SEQ ID NO: 157 - protein, 9 aa, Artificial Sequence
  PM 76-B10.11 CC VL CDR3
QQYDQQLIT

SEQ ID NO: 158 - protein, 121 aa, Artificial Sequence
  PM 76-B10.11 CC VH
QVQLVESGGGLVKPGESLRLSCAASGFTFSDYYMYWVRQAPGKGLEWVAIISDGGYYTYYSDIIKGRFTISRDNAKNSLYLQMNSLKAEDTAVYYCARGFPLLRHGAMDYWGQGTLVTVSS

SEQ ID NO: 159 - protein, 107 aa, Artificial Sequence
  PM 76-B10.11 CC VL
DIQMTQSPSSLSASVGDRVTITCKASQNVDTNVAWYQQKPGQAPKSLIYSASYVYWDVPSRFSGSASGTDFTLTISSVQSEDFATYYCQQYDQQLITFGGGTKLEIK

SEQ ID NO: 160 - protein, 243 aa, Artificial Sequence
  PM 76-B10.11 CC scFv
QVQLVESGGGLVKPGESLRLSCAASGFTFSDYYMYWVRQAPGKGLEWVAIISDGGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDTNVAWYQQKPGQAPKSLIYSASYVYWDVPSRFSGSASGTDFTLTISSVQSEDFATYYCQQYDQQLITFGGGTKLEIK

SEQ ID NO: 161 - protein, 498 aa, Artificial Sequence
  PM 76-B10.11 CC x I2C0 bispecific molecule
QVQLVESGGGLVKPGESLRLSCAASGFTFSDYYMYWVRQAPGKGLEWVAIISDGGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDTNVAWYQQKPGQAPKSLIYSASYVYWDVPSRFSGSASGTDFTLTISSVQSEDFATYYCQQYDQQLITFGGGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 162 - protein, 986 aa, Artificial Sequence
  PM 76-B10.11 CC x I2C0-scFc bispecific HLE molecule
QVQLVESGGGLVKPGESLRLSCAASGFTFSDYYMYWVRQAPGKGLEWVAIISDGGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDTNVAWYQQKPGQAPKSLIYSASYVYWDVPSRFSGSASGTDFTLTISSVQSEDFATYYCQQYDQQLITFGGGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 163 - protein, 984 aa, Artificial Sequence
  PM 76-B10.11 CC x I2C0-scFc_delGK bispecific HLE molecule
QVQLVESGGGLVKPGESLRLSCAASGFTFSDYYMYWVRQAPGKGLEWVAIISDGGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDTNVAWYQQKPGQAPKSLIYSASYVYWDVPSRFSGSASGTDFTLTISSVQSEDFATYYCQQYDQQLITFGGGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 164 - protein, 498 aa, Artificial Sequence
  PM 76-B10.11 CC x I2C0 CC (103/43)-scFc bispecific molecule
QVQLVESGGGLVKPGESLRLSCAASGFTFSDYYMYWVRQAPGKGLEWVAIISDGGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDTNVAWYQQKPGQAPKSLIYSASYVYWDVPSRFSGSASGTDFTLTISSVQSEDFATYYCQQYDQQLITFGGGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYCGQGTLVTVSSGGGGSGGGGSGGGGSQTVVTQEPSLTVSPGGTVTLTCGSSTGAVTSGNYPNWVQQKPGQCPRGLIGGTKFLAPGTPARFSGSLLGGKAALTLSGVQPEDEAEYYCVLWYSNRWVFGGGTKLTVL

SEQ ID NO: 165 - protein, 986 aa, Artificial Sequence
  PM 76-B10.11 CC x I2C0 CC (103/43)-scFc bispecific HLE molecule
QVQLVESGGGLVKPGESLRLSCAASGFTFSDYYMYWVRQAPGKGLEWVAIISDGGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDTNVAWYQQKPGQAPKSLIYSASYVYWDVPSRFSGSASGTDFTLTISSVQSEDFATYYCQQYDQQLITFGGGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYCGQGTLVTVSSGGGGSGGGGSGGGGSQTVVTQEPSLTVSPGGTVTLTCGSSTGAVTSGNYPNWVQQKPGQCPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 166 - protein, 984 aa, Artificial Sequence
  PM 76-B10.11 CC x I2C0 CC (103/43)-scFc_delGK bispecific HLE molecule
QVQLVESGGGLVKPGESLRLSCAASGFTFSDYYMYWVRQAPGKGLEWVAIISDGGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDTNVAWYQQKPGQAPKSLIYSASYVYWDVPSRFSGSASGTDFTLTISSVQSEDFATYYCQQYDQQLITFGGGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYCGQGTLVTVSSGGGGSGGGGSGGGGSQTVVTQEPSLTVSPGGTVTLTCGSSTGAVTSGNYPNWVQQKPGQCPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 167 - protein, 5 aa, Artificial Sequence
  PM 76-B10.11 CC x I2C0-scFc VH CDR1
DYYMY

SEQ ID NO: 168 - protein, 17 aa, Artificial Sequence
  PM 76-B10.11 CC x I2C0-scFc VH CDR2
IISDGGYYTYYSDIIKG

SEQ ID NO: 169 - protein, 12 aa, Artificial Sequence
  PM 76-B10.11 CC x I2C0-scFc VH CDR3
GFPLLRHGAMDY

SEQ ID NO: 170 - protein, 11 aa, Artificial Sequence
  PM 76-B10.11 CC x I2C0-scFc VL CDR1
KASQNVDTNVA

SEQ ID NO: 171 - protein, 7 aa, Artificial Sequence
  PM 76-B10.11 CC x I2C0-scFc VL CDR2
SASYVYW

SEQ ID NO: 172 - protein, 9 aa, Artificial Sequence
  PM 76-B10.11 CC x I2C0-scFc VL CDR3
QQYDQQLIT

SEQ ID NO: 173 - protein, 121 aa, Artificial Sequence
  PM 76-B10.11 CC x I2C0-scFc VH
QVQLVESGGGLVKPGESLRLSCAASGFTFSDYYMYWVRQAPGKCLEWVAIISDGGYYTYYSDIIKGRFTISRDNAKNSLYLQMNSLKAEDTAVYYCARGFPLLRHGAMDYWGQGTLVTVSS

SEQ ID NO: 174 - protein, 107 aa, Artificial Sequence
  PM 76-B10.11 CC x I2C0-scFc VL
DIQMTQSPSSLSASVGDRVTITCKASQNVDTNVAWYQQKPGQAPKSLIYSASYVYWDVPSRFSGSASGTDFTLTISSVQSEDFATYYCQQYDQQLITFGCGTKLEIK

SEQ ID NO: 175 - protein, 243 aa, Artificial Sequence
  PM 76-B10.11 CC x I2C0-scFc scFv
QVQLVESGGGLVKPGESLRLSCAASGFTFSDYYMYWVRQAPGKCLEWVAIISDGGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDTNVAWYQQKPGQAPKSLIYSASYVYWDVPSRFSGSASGTDFTLTISSVQSEDFATYYCQQYDQQLITFGCGTKLEIK

SEQ ID NO: 176 - protein, 498 aa, Artificial Sequence
  PM 76-B10.11 CC x I2C0-scFc bispecific molecule
QVQLVESGGGLVKPGESLRLSCAASGFTFSDYYMYWVRQAPGKCLEWVAIISDGGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDTNVAWYQQKPGQAPKSLIYSASYVYWDVPSRFSGSASGTDFTLTISSVQSEDFATYYCQQYDQQLITFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 177 - protein, 986 aa, Artificial Sequence
  PM 76-B10.11 CC x I2C0-scFc bispecific HLE molecule
QVQLVESGGGLVKPGESLRLSCAASGFTFSDYYMYWVRQAPGKCLEWVAIISDGGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDTNVAWYQQKPGQAPKSLIYSASYVYWDVPSRFSGSASGTDFTLTISSVQSEDFATYYCQQYDQQLITFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 178 - protein, 984 aa, Artificial Sequence
  PM 76-B10.11 CC x I2C0-scFc_delGK bispecific HLE molecule
QVQLVESGGGLVKPGESLRLSCAASGFTFSDYYMYWVRQAPGKCLEWVAIISDGGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDTNVAWYQQKPGQAPKSLIYSASYVYWDVPSRFSGSASGTDFTLTISSVQSEDFATYYCQQYDQQLITFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 179 - protein, 498 aa, Artificial Sequence
  PM 76-B10.11 CC x I2C0 CC (103/43)-scFc bispecific molecule
QVQLVESGGGLVKPGESLRLSCAASGFTFSDYYMYWVRQAPGKCLEWVAIISDGGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDTNVAWYQQKPGQAPKSLIYSASYVYWDVPSRFSGSASGTDFTLTISSVQSEDFATYYCQQYDQQLITFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYCGQGTLVTVSSGGGGSGGGGSGGGGSQTVVTQEPSLTVSPGGTVTLTCGSSTGAVTSGNYPNWVQQKPGQCPRGLIGGTKFLAPGTPARFSGSLLGGKAALTLSGVQPEDEAEYYCVLWYSNRWVFGGGTKLTVL

SEQ ID NO: 180 - protein, 986 aa, Artificial Sequence
  PM 76-B10.11 CC x I2C0 CC (103/43)-scFc bispecific HLE molecule
QVQLVESGGGLVKPGESLRLSCAASGFTFSDYYMYWVRQAPGKCLEWVAIISDGGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDTNVAWYQQKPGQAPKSLIYSASYVYWDVPSRFSGSASGTDFTLTISSVQSEDFATYYCQQYDQQLITFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYCGQGTLVTVSSGGGGSGGGGSGGGGSQTVVTQEPSLTVSPGGTVTLTCGSSTGAVTSGNYPNWVQQKPGQCPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 181 - protein, 984 aa, Artificial Sequence
  PM 76-B10.11 CC x I2C0 CC (103/43)-scFc_delGK bispecific HLE molecule
QVQLVESGGGLVKPGESLRLSCAASGFTFSDYYMYWVRQAPGKCLEWVAIISDGGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDTNVAWYQQKPGQAPKSLIYSASYVYWDVPSRFSGSASGTDFTLTISSVQSEDFATYYCQQYDQQLITFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYCGQGTLVTVSSGGGGSGGGGSGGGGSQTVVTQEPSLTVSPGGTVTLTCGSSTGAVTSGNYPNWVQQKPGQCPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 182 - protein, 5 aa, Artificial Sequence
  CD70_21D_CC VH CDR1
TYAMS

SEQ ID NO: 183 - protein, 17 aa, Artificial Sequence
  CD70_21D_CC VH CDR2
AISGSGGRTFYAESVEG

SEQ ID NO: 184 - protein, 11 aa, Artificial Sequence
  CD70_21D_CC VH CDR3
HDYSNYPYFDY

SEQ ID NO: 185 - protein, 12 aa, Artificial Sequence
  CD70_21D_CC VL CDR1
RASQSVRSTYLA

SEQ ID NO: 186 - protein, 7 aa, Artificial Sequence
  CD70_21D_CC VL CDR2
GASSRAT

SEQ ID NO: 187 - protein, 9 aa, Artificial Sequence
  CD70_21D_CC VL CDR3
QQYGDLPFT

SEQ ID NO: 188 - protein, 120 aa, Artificial Sequence
  CD70_21D_CC VH
EVQLLESGGGMVQPGGSLRLSCAASGFTFSTYAMSWVRQAPGKCLEWVSAISGSGGRTFYAESVEGRFTISRDNSKNTLYLQMNSLRAEDTAVYYCAKHDYSNYPYFDYWGQGTLVTVSS

SEQ ID NO: 189 - protein, 108 aa, Artificial Sequence
  CD70_21D_CC VL
EIVLTQSPGTLSLSPGERATLSCRASQSVRSTYLAWYQQKPGQAPRLLIYGASSRATGIPDRFSGSGSGTDFTLTISRLEPEDFAVYSCQQYGDLPFTFGCGTKLEIK

SEQ ID NO: 190 - protein, 986 aa, Artificial Sequence
  CD70_21D_CCx12C scFc
EVQLLESGGGMVQPGGSLRLSCAASGFTFSTYAMSWVRQAPGKCLEWVSAISGSGGRTFYAESVEGRFTISRDNSKNTLYLQMNSLRAEDTAVYYCAKHDYSNYPYFDYWGQGTLVTVSSGGGGSGGGGSGGGGSEIVLTQSPGTLSLSPGERATLSCRASQSVRSTYLAWYQQKPGQAPRLLIYGASSRATGIPDRFSGSGSGTDFTLTISRLEPEDFAVYSCQQYGDLPFTFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 191 - protein, 10 aa, Artificial Sequence
  hinge sequence
DKTHTCPPCP

SEQ ID NO: 192 - protein, 12 aa, Artificial Sequence
  hinge sequence
ERKCCVECPPCP

SEQ ID NO: 193 - protein, 16 aa, Artificial Sequence
  hinge sequence
ELKTPLDTTHTCPRCP

SEQ ID NO: 194 - protein, 17 aa, Artificial Sequence
  hinge sequence
ELKTPLGDTTHTCPRCP

SEQ ID NO: 195 - protein, 12 aa, Artificial Sequence
  hinge sequence
ESKYGPPCPSCP

SEQ ID NO: 196 - protein, 15 aa, Artificial Sequence
  hinge sequence
EPKSCDKTHTCPPCP

SEQ ID NO: 197 - protein, 5 aa, Artificial Sequence
  linker
GGGGS

SEQ ID NO: 198 - protein, 6 aa, Artificial Sequence
  His-tag
HHHHHH

SEQ ID NO: 199 - protein, 15 aa, Artificial Sequence
  linker
GGGGSGGGGSGGGGS

(19) United States
(12) Patent Application Publication
(10) Pub. No.: US 2024/0336685 A1
(43) Pub. Date: Oct. 10, 2024

(54) METHODS FOR REDUCING AGGREGATION OF BISPECIFIC ANTIBODIES

(71) Applicant: AMGEN INC., Thousand Oaks, CA (US)

(72) Inventors: Bharadwaj Jagannathan, Thousand Oaks, CA (US); Joon Huh, Thousand Oaks, CA (US); Michael J. Treuheit, Newbury Park, CA (US); Daxian Shan, Thousand Oaks, CA (US)

(21) Appl. No.: 17/281,896

(22) PCT Filed: Sep. 27, 2019
(86) PCT No.: PCT/US19/53462

§ 371 Date: Mar. 31, 2021

Related U.S. Application Data
(60) Provisional application No. 62/739,542, filed on Oct. 1, 2018.

(51) Int. Cl. C07K 16/28

(52) U.S. Cl. CPC C07K 16/2809; C07K 16/2803; C07K 16/2878; C07K 2317/10; C07K 2317/31

(57) ABSTRACT

The invention disclosed herein relates to methods for reducing aggregation of bispecific antibodies, for example reducing aggregation of bispecific T cell engager (BiTE) antibody constructs, resulting from storage under frozen conditions by holding the antibodies at certain temperatures after thaw.

This application is a U.S. National Stage of International Application No. PCT/US2019/0053462, filed Sep. 27, 2019, which claims the benefit of U.S. Provisional Application No. 62/739,542, filed on Oct. 1, 2018, the disclosures of which are incorporated herein by reference.

Incorporated by reference in its entirety is a computer-readable nucleotide/amino acid sequence listing submitted concurrently herewith and identified as follows: 55258_SubSeqListing.txt; Size: 394,750 bytes; Created: Apr. 22, 2024.

### FIELD OF THE INVENTION

The present application relates to methods for reducing aggregation of bispecific antibodies. More specifically, the present application relates to methods for reducing aggregation of bispecific antibodies disclosed herein such as bispecific T cell engager antibodies resulting from storage under frozen conditions.

### BACKGROUND OF THE INVENTION

Therapeutic proteins such as antibodies are an important class of medicines serving patients. Typically, therapeutic proteins are produced in Eukaryotic cells and purified in large quantities. Because proteins are sensitive to temperature change, drug substance of these therapeutic proteins are stored and shipped under frozen conditions at temperatures ranging from −20° C. to −80° C. Freezing extends shelf life of therapeutic proteins and provides flexibility for scheduling the final formulation and fill of the proteins into the marketed product packaging. Operational infrastructure and logistics for storage and shipment are favorable at higher temperatures in the range.

The freeze/thaw process can be a source of stress for proteins. For example, water crystalizes during the freezing process of a protein drug substance, which can lead the protein molecules to reach concentration levels several times that of the starting level. The concentration change can result in a loss of a protein's thermodynamic stability, leading to unfolding events and causing aggregation. See e.g., 18 Lam Philippe et al., Quality by Design for Biopharmaceutical Drug Product Development, pp. 159-189 (Jameel F. et al. ed., 2015). Similar stresses may be involved in the thawing process, and they may be exacerbated due to the higher temperatures required to melt the ice in a timely fashion. Id. Thus, protein aggregates (e.g., high molecular weight (HMW) aggregates) may result from freeze/thaw processes when drug substances comprising therapeutic proteins are stored and/or shipped under frozen conditions. However, the presence of aggregates in drug substances is undesirable as the aggregates may negatively impact the stability, immunogenicity and potency of the proteins. There is a need for methods of decreasing protein aggregation resulting from storage under frozen conditions.

### SUMMARY OF THE INVENTION

Provided herein are methods for reducing aggregation of bispecific antibodies, in particular, bispecific T cell engager antibodies (BiTEs), that results from storage under frozen conditions. The present application is directed to the surprising findings that holding the bispecific antibodies at certain temperatures for a period of time after thaw reduces aggregates formed during storage under frozen conditions.

In one embodiment, disclosed herein is a method for reducing aggregates of a bispecific antibody comprising holding a thawed bispecific antibody at a temperature of from about 5° C. to about 45° C.′ for at least 4 hours, wherein the bispecific antibody has been stored at a temperature of from about −20° C. to about −40° C. before thaw. In one embodiment, the thawed bispecific antibody is held at the temperature for a period of from about 4 hours to about 96 hours. In one embodiment, the thawed bispecific antibody is held at a temperature of from about 10° C. to about 30° C. for a period of from about 10 hours to about 48 hours. In one embodiment, the thawed bispecific antibody is held at a temperature of from about 10° C. to about 30° C. for a period of from 8 hours to 48 hours.

In one embodiment, the bispecific antibody is thawed at a temperature of from about 5° C. to about 45° C. In one embodiment, the bispecific antibody has been stored at a temperature of from about −20° C. to about −35° C. before thaw, in another embodiment, the bispecific antibody has been stored at about −30° C. before thaw. In one embodiment, the aggregates comprise high molecule weight (HMW) aggregates. In one embodiment, the HMW aggregates comprise dimers of the bispecific antibody. In one embodiment, the bispecific antibody comprises less than about 1% of the HMW aggregates after the holding period, in another embodiment, the bispecific antibody comprises less than about 0.5% of the HMW aggregates after the holding period.

In one embodiment, disclosed herein is a method for preparing a composition comprising a bispecific antibody, the method comprises thawing a drug substance comprising a bispecific antibody that has been stored at a temperature of from about −20° C. to about −40° C., and holding the thawed drug substance comprising the bispecific antibody at a temperature of from about 5° C. to about 45° C. for at least 4 hours. In one embodiment, the drug substance is held at the temperature for a period of from about 4 hours to about 96 hours. In another embodiment, the drug substance is held at a temperature of from about 10° C. to about 30° C. for a period of from about 10 hours to about 48 hours. In another embodiment, the drug substance is held at a temperature of from about 10° C. to about 30° C. for a period of from 8 hours to 48 hours. In one embodiment, the drug substance is thawed at a temperature of from about 5° C. to about 45° C. In one embodiment, the drug substance has been stored at a temperature of from about −20° C. to about −35° C., in another embodiment, the drug substance has been stored at about −30° C. In one embodiment, the method for preparing the composition further comprises filtering the drug substance, in another embodiment, the method for preparing the composition further comprises aliquoting the composition into a drug product form. In one embodiment, the composition is a pharmaceutical composition comprising the bispecific antibody.

In one embodiment, disclosed herein is a method for preparing a composition comprising a bispecific antibody, the method comprises holding a thawed drug substance comprising a bispecific antibody at a temperature of from about 5° C. to about 45° C. for at least 4 hours, wherein the drug substance has been frozen at or above the glass transition temperature (Tg′) of the drug substance before thaw. In one embodiment, the drug substance has been frozen at a temperature of from about −10° C. to at or above the glass transition temperature of the drug substance, in another embodiment, the drug substance has been frozen at about −32° C. In one embodiment, the drug substance is held at the temperature for a period of from about 4 hours to about 96 hours. In one embodiment, the drug substance is thawed at a temperature of from about 5° C. to about 45° C. In another embodiment, the drug substance is held at the same temperature as the temperature at which the drug substance is thawed. In another embodiment, the drug substance is thawed and held at the same temperature of from about 15° C. to about 30° C. for a period of from 30 hours to 50 hours. In another embodiment, the method for preparing the composition further comprises aliquoting the composition into a drug product form. In another embodiment, the method for preparing the composition further comprises lyophilizing the composition. In yet another embodiment, the method for preparing the composition further comprises spray drying the composition.

In one embodiment, the drug substance comprises less than about 1% of HMW aggregates after the holding period, in another embodiment, the drug substance comprises less than about 0.5% of the HMW aggregates. In one embodiment, the HMW aggregates comprise dimers of the bispecific antibody.

In one embodiment, the drug substance comprising the bispecific antibody at a concentration of from about 0.05 mg/mL to about 20 mg/mL.

In one embodiment, the bispecific antibody is a bispecific T cell engager antibody (BiTE). In one embodiment, the bispecific antibody comprises a first binding domain that binds to a target cell surface antigen, and a second binding domain that binds to human CD3, and wherein the bispecific antibody is in (scFv) 2 format. In one embodiment, the bispecific antibody comprises a first binding domain that binds to a target cell surface antigen selected from CD19, CD33 or BCMA, and a second binding domain that binds to human CD3, and wherein the bispecific antibody is in (scFv) 2 format.

In one embodiment, the bispecific antibody comprises a first binding domain and a second binding domain, wherein the first binding domain comprises a VH region and a VL region, and wherein: the VH comprises the amino acid sequence of SEQ ID NO: 77 and the VL comprises the amino acid sequence of SEQ ID NO: 78; or the VH comprises the amino acid sequence of SEQ ID NO: 28 and the VL comprises the amino acid sequence of SEQ ID NO: 32 or 33; or the VH comprises the amino acid sequence of SEQ ID NO: 132 and the VL comprises the amino acid sequence of SEQ ID NO: 133. In another embodiment, the bispecific antibody comprises the amino acid sequence of SEQ ID NO: 17, 40 or 135.

In one embodiment, the bispecific antibody is a BiTE, wherein the BiTE further comprises a third domain comprising two polypeptide monomers, each comprises a hinge, a CH2 and a CH3 domain, wherein the two polypeptide monomers are linked to each other via a peptide linker. In another embodiment, the third domain comprises in an amino to carboxyl order hinge-CH2-CH3-linker-hinge-CH2-CH3. In one embodiment, the third domain is a half-life extended (HLE) domain.

In one embodiment, the bispecific antibody comprises a first binding domain, a second binding domain, and a third domain, wherein the first binding domain binds to at least one target cell surface antigen selected from CD19, CD33, EGFRvIII, MSLN, CDH19, FLT3, DLL3, CDH3, CD70, BCMA or PSMA, the second binding domain binds to human CD3, and the third domain comprises two polypeptide monomers, each comprises a hinge, a CH2 and a CH3 domain, wherein the two polypeptide monomers are linked to each other via a peptide linker.

In one embodiment, the bispecific antibody comprises a first binding domain, a second binding domain, and a third domain, the first binding domain comprises a VH region and a VL region and binds to at least one target cell surface antigen selected from CD19, CD33, EGFRvIII, MSLN, CDH19, FLT3, DLL3, CDH3, CD70, BCMA or PSMA, the second binding domain binds to human CD3, and the third domain comprises two polypeptide monomers, each comprises a hinge, a CH2 and a CH3 domain, wherein the two polypeptide monomers are linked to each other via a peptide linker, and wherein: (a) the VH comprises the amino acid sequence of SEQ ID NO: 108 and the VL comprises the amino acid sequence of SEQ ID NO: 109; or (b) the VH comprises the amino acid sequence of SEQ ID NO: 27 and the VL comprises the amino acid sequence of SEQ ID NO: 32; or (c) the VH comprises the amino acid sequence of SEQ ID NO: 48 and the VL comprises the amino acid sequence of SEQ ID NO: 49; or (d) the VH comprises the amino acid sequence of SEQ ID NO: 59 and the VL comprises the amino acid sequence of SEQ ID NO: 60; or (c) the VH comprises the amino acid sequence of SEQ ID NO: 77 and the VL comprises the amino acid sequence of SEQ ID NO: 78; or (f) the VH comprises the amino acid sequence of SEQ ID NO: 108 and the VL comprises the amino acid sequence of SEQ ID NO: 112; or (g) the VH comprises the amino acid sequence of SEQ ID NO: 89 and the VL comprises the amino acid sequence of SEQ ID NO: 90; or (h) the VH comprises the amino acid sequence of SEQ ID NO: 100 and the VL comprises the amino acid sequence of SEQ ID NO: 101; or (i) the VH comprises the amino acid sequence of SEQ ID NO: 121 and the VL comprises the amino acid sequence of SEQ ID NO: 122; or (j) the VH comprises the amino acid sequence of SEQ ID NO: 188 and the VL comprises the amino acid sequence of SEQ ID NO: 189; or (k) the VH comprises the amino acid sequence of SEQ ID NO: 132 and the VL comprises the amino acid sequence of SEQ ID NO: 133; or (1) the VH comprises the amino acid sequence of SEQ ID NO: 173 and the VL comprises the amino acid sequence of SEQ ID NO: 174.

In one embodiment, the bispecific antibody comprises a first binding domain, a second binding domain, and a third domain, the first binding domain binds to at least one target cell surface antigen selected from CD19, CD33, EGFRvIII, MSLN, CDH19, FLT3, DLL3, CDH3, CD70, BCMA or PSMA, the second binding domain binds to human CD3, and the third domain comprises two polypeptide monomers, each comprises a hinge, a CH2 and a CH3 domain, wherein the two polypeptide monomers are linked to each other via a peptide linker, and wherein the bispecific antibody comprising or consisting of the amino acid sequence selected from SEQ ID NO: 63, 114, 41, 82, 136, 104, 93, 177, 125, 190 or 52.

In one embodiment, the bispecific antibody is a bispecific masked antigen binding protein. In one embodiment, the bispecific masked antigen binding protein comprises (a) a first antibody or antigen binding fragment thereof (AB1) that binds to a first antigen and a masking domain (MD1) coupled to AB1, wherein the MD1 comprises (1) a first masking peptide (MP1) that inhibits or reduces the binding of AB1 to its antigen, and (2) a protein recognition site (PR1), wherein binding to or cleavage of the PR1 by a protein or a protease increases AB1 binding to its antigen; (b) a second antibody or antigen binding fragment there of (AB2) that binds to a second antigen, and a second masking domain (MD2) coupled to AB2, wherein the MD2 comprises: (1) a second masking peptide (MPs) that inhibits or reduces the binding of AB2 to its antigen, and (2) a second protein recognition site (PR2), wherein binding to or cleavage of the PR2 by a protein or a protease increases AB2 binding to its antigen. In one embodiment, the PR1 and PR2 comprise the same protein recognition sequence. In another embodiment, the AB1 binds to human CD3 and the AB2 binds to human EGFR.

### BRIEF DESCRIPTION OF THE DRAWINGS

FIG. 1 shows the increase of aggregate levels (ΔHMW %) after various HLE BITE molecules being stored at −20° C. for one month.

FIG. 2A shows the increase of aggregate levels (ΔHMW %) of the DLL3×CD3 HLE BITE after storage at −20° C. for one month in compositions having different pH.

FIG. 2B shows the increase of aggregate levels (ΔHMW %) of the DLL3×CD3 HLE BITE after storage at different temperatures for one month.

FIGS. 3A, 3B, 3C and 3D show that HMW aggregate levels increased in various HLE BiTEs after storage at −20° C. or −30° C., and that holding at room temperature for 24 hours after thaw reduced the aggregate levels.

FIG. 4 shows hold time and hold temperature dependence of the decrease in HMW levels of BiTE Molecules.

FIG. 5 shows the stabilizing effect of benzyl alcohol.

### DETAILED DESCRIPTION OF THE INVENTION

### Methods

Described herein are methods for reducing aggregates of bispecific antibodies, in particular, aggregates formed when the bispecific antibodies are stored under frozen conditions. As used herein, the term “aggregate(s)” or “aggregation” refers to an association of two or more molecules. In certain embodiments, the aggregates are high molecular weight (HMW) aggregates that have molecular weight and/or dimensions larger than that of the non-aggregated molecule. In certain embodiments, the aggregates comprise an association of two or more antibody molecules. In certain embodiments, the aggregates comprise an association of two or more bispecific antibody molecules including dimers of the bispecific antibody. In certain embodiments, the bispecific antibody is a bispecific T cell engager antibody (BiTE).

The presence and/or level of aggregates can be determined by techniques known in the art such as techniques that determine the size of a molecule, e.g., size exclusion chromatography, cation exchange chromatography, X-ray diffraction, modulated differential scanning calorimetry (mDSC), and non-denaturing polyacrylamide gel electrophoresis (PAGE). In one embodiment, the presence and/or level of protein aggregates are determined by size exclusion HPLC (SE-HPLC). In another embodiment, the presence and/or level of protein aggregates are determined by size exclusion ultra HPLC (SE-UHPLC).

In one aspect, disclosed herein is a method for reducing aggregates of a bispecific antibody, the method comprises holding a thawed bispecific antibody at a certain temperature for at least 4 hours, wherein the bispecific antibody has been stored under frozen conditions before thaw. As used herein, the term “a thawed bispecific antibody” or “a thawed drug substance comprising a bispecific antibody” is understood to refer to a liquid state bispecific antibody or a liquid state drug substance comprising a bispecific antibody that comes from a frozen state as a result of exposure to warmth. In certain embodiments, the thawed bispecific antibody or the thawed drug substance comprising a bispecific antibody is a bispecific antibody or a drug substance comprising a bispecific antibody in a liquid state that is free of or substantially free of frozen material.

In certain embodiments, the bispecific antibody is a drug substance. As used herein, the term “drug substance” is understood to refer to a recombinant protein (e.g., a bispecific antibody) that has been sufficiently purified or isolated from contaminating proteins, lipids, and nucleic acids (e.g., contaminating proteins, lipids, and nucleic acids present in a liquid culture medium or from a host cell (e.g., from a mammalian, yeast, or bacterial host cell)) and biological contaminants (e.g., viral and bacterial contaminants), and can be formulated into a pharmaceutical composition without further substantial purification and/or decontamination steps. The term drug substance encompasses a composition comprising a sufficiently purified recombinant protein (e.g., a bispecific antibody) and one or more pharmaceutically suitable excipients.

In certain embodiments, the thawed drug substance is a composition comprising one or more excipients in addition to the recombinant protein (e.g., a bispecific antibody). Excipients suitable for suitable for pharmaceutical compositions can be used. Exemplary excipients include buffers (e.g., an acetate buffer, a glutamate buffer, a citrate buffer, a lactic buffer, a succinate buffer, a tartrate buffer, a fumarate buffer, a maleate buffer, a histidine buffer, or a phosphate buffer), saccharides (e.g., glucose, galactose, fructose, xylose, sucrose, lactose, maltose, trehalose, sorbitol, mannitol or xylitol), and surfactants (e.g., polysorbate 20 or polysorbate 80). In certain embodiments, the thawed drug substance is a composition comprising a recombinant protein (e.g., a bispecific antibody), a buffer (e.g., a glutamate buffer or a citrate buffer), a saccharide (e.g., sucrose) and optionally a surfactant (e.g., polysorbate 80). The pH of the thawed drug substance can be in the range of from about 3.0 to 7.0 or from about 4.0 to about 6.0.

In certain embodiments, the frozen bispecific antibody is thawed by exposing to elevated temperatures. In certain embodiments, the bispecific antibody is thawed at a temperature of from about 0° C. to about 50° C., or from about 0° C. to about 40° C., or from about 0° C. to about 30° C., or from about 5° C. to about 45° C., or from about 5° C. to about 30° C. or from about 10° C. to about 50° C., or from about 10° C. to about 40° C., or from about 10° C. to about 30° C., or from about 15° C. to about 50° C., or from about 15° C. to about 40° C., or from about 15° C. to about 30° C., or from about 20° C. to about 30° C., or from about 25° C. to about 30° C. In certain embodiments, the bispecific antibody is thawed at a temperature of from about 0° C. to about 25° C., or from about 5° C. to about 25° C., or from about 10° C. to about 25° C., or from about 15° C. to about 25° C. or from about 20° C. to about 25° C. In certain embodiments, the bispecific antibody is thawed at a temperature of about 0° C. about 5° C., about 10° C., about 15° C., about 20° C., about 25° C., or about 30° C., or about 40° C., or about 45° C., or about 50° C. As can be appreciated by one of ordinary skill in the art, the bispecific antibody may be mixed gently during thaw to ensure homogeneous distribution of temperature and/or disrupting concentration gradients formed during thaw. Gentle mixing may be achieved by, for example, using a tilting shaker or gently inverting the container of the bispecific antibody. Alternatively, the bispecific antibody may be mixed gently after thaw.

In certain embodiments, the method comprises holding the thawed bispecific antibody at a certain temperature for at least 4 hours. In certain embodiments, the holding temperature is from about 0° C. to about 50° C., or from about 0° C. to about 40° C., or from about 5° C. to about 50° C. or from about 5° C. to about 45° C. or from about 5° C. to about 40° C., or from about 5° C. to about 30° C., or from about 10° C. to about 50° C., or from about 10° C. to about 45° C., or from about 10° C. to about 40° C., or from about 10° C. to about 30° C., or from about 15° C. to about 40° C. or from about 15° C. to about 30° C. In certain embodiment, the holding temperature is from about 15° C. to about 25° C. In certain embodiments, the holding temperature is about 5° C., or about 10° C., or about 15° C., or about 17° C., or about 19° C., or about 20° C., or about 23° C., or about 25° C., or about 27° C. or about 30° C., or about 35° C., or about 40° C. or about 45° C. In certain embodiments, the thawed bispecific antibody is held at any one of the above temperatures for a period of from about 4 hours to about 120 hours, or from about 4 hours to about 96 hours, or from about 4 hours to about 72 hours, or from about 4 hours to about 48 hours, or from about 4 hours to about 24 hours, or from about 10 hours to about 120 hours, or from about 10 hours to about 96 hours, or from about 10 hours to about 72 hours, or from about 10 hours to about 48 hours, or from about 10 hours to about 24 hours, or from about 24 hours to about 120 hours, or from about 24 hours to about 100 hours, or from about 24 hours to about 96 hours, or from about 24 hours to about 72 hours, or from about 24 hours to about 48 hours, or from about 48 hours to about 100 hours, or from about 48 hours to about 96 hours, or from about 48 hours to about 72 hours, or from about 72 hours to about 100 hours, or from about 72 hours to about 96 hours.

In certain embodiments, the thawed bispecific antibody is held at a temperature of from about 5° C. to about 45° C. for a period of from about 4 hours to about 120 hours, or for a period of from about 4 hours to about 100 hours, or for a period of from about 4 hours to about 96 hours, or for a period of from about 4 hours to about 72 hours, or for a period of from about 4 hours to about 48 hours, or for a period of from about 4 hours to about 24 hours. In certain embodiments, the thawed bispecific antibody is held at a temperature of from about 10° C. to about 30° C. for a period of from about 4 hours to about 24 hours, or for a period of from about 10 hours to about 120 hours, or for a period of from about 10 hours to about 96 hours, or for a period of from about 10 hours to about 72 hours, or for a period of from about 10 hours to about 48 hours, or for a period of from about 10 hours to about 24 hours. In certain embodiments, the thawed bispecific antibody is held at a temperature of from about 30° C. to about 45° C. for a period of from about 4 hours to about 24 hours, or for a period of from about 4 hours to about 10 hours. In certain embodiments, the thawed bispecific antibody is held at a temperature of from about 15° C. to about 45° C. for a period of from about 4 hours to about 120 hours, or a period of from about 4 hours to about 50 hours, or for a period of from about 4 hours to about 24 hours, or for a period of from about 8 hours to about 50 hours, or a period of from about 15 hours to about 50 hours, or for a period of from about 4 hours to about 100 hours, or for a period of from about 8 hours to about 100 hours, or for a period of from about 15 hours to about 100 hours, or for a period of from about 24 hours to about 96 hours, or for a period of from about 24 hours to about 72 hours, or for a period of from about 24 hours to about 48 hours, or for a period of from about 48 hours to about 100 hours, or for a period of from about 48 hours to about 96 hours, or for a period of from about 48 hours to about 72 hours, or for a period of from about 72 hours to about 100 hours, or for a period of from about 72 hours to about 96 hours. In certain embodiments, the thawed bispecific antibody is held at a temperature of from about 15° C. to about 30° C. for a period of from about 4 hours to about 24 hours, or for a period of from about 10 hours to about 120 hours, or for a period of from about 10 hours to about 96 hours, or for a period of from about 10 hours to about 72 hours, or for a period of from about 10 hours to about 48 hours, or for a period of from about 10 hours to about 24 hours, for a period of from about 24 hours to about 96 hours, or for a period of from about 24 hours to about 72 hours, or for a period of from about 24 hours to about 48 hours, or for a period of from about 48 hours to about 100 hours, or for a period of from about 48 hours to about 96 hours, or for a period of from about 48 hours to about 72 hours, or for a period of from about 72 hours to about 100 hours, or for a period of from about 72 hours to about 96 hours. In certain embodiments, the thawed bispecific antibody is held at a temperature of from about 15° C. to about 25° C. for a period of from about 4 hours to about 24 hours, or for a period of from about 10 hours to about 48 hours, or for a period of from about 15 hours to about 30 hours, or for a period of from about 15 hours to about 24 hours, or for a period of from about 24 hours to about 120 hours, or for a period of from about 24 hours to about 96 hours, or for a period of from about 24 hours to about 72 hours, or for a period of from about 24 hours to about 48 hours, or for a period of from about 48 hours to about 100 hours, or for a period of from about 48 hours to about 96 hours, or for a period of from about 48 hours to about 72 hours, or for a period of from about 72 hours to about 100 hours, or for a period of from about 72 hours to about 96 hours. In another embodiment, the thawed bispecific antibody is held at a temperature of from about 15° C. to about 30° C. for a period of about 10 hours, or about 15 hours, or about 20 hours, or about 24 hours, or about 36 hours, or about 48 hours, or about 60 hours, or about 72 hours, or about 84 hours, or about 96 hours, or about 120 hours.

The bispecific antibody has been stored under frozen conditions before thaw. In certain embodiments, the bispecific antibody has been stored at a temperature of from about −20° C. to about −50° C. In certain embodiments, the bispecific antibody has been stored at a temperature of from about −20° C. to about −40° C. In certain embodiments, the bispecific antibody has been stored at a temperature of from about −25° C. to about −35° C. In certain embodiments, the bispecific antibody has been stored at a temperature of about −20° C., about −30° C., about −35° C., about −40° C., or about-50° C. In certain embodiments, the bispecific antibody has been stored at a temperature of about −30° C. In certain embodiments, the bispecific antibody has been stored at a temperature of from 0° C. to at or above the glass transition temperature (Tg′) of the antibody or the Tg′ of the composition comprising the antibody. In certain embodiments, the bispecific antibody has been stored at a temperature of from about −10° C. to at or above the Tg′ of the antibody or the Tg′ of the composition comprising the antibody.

In certain embodiments, the bispecific antibody has been stored at a temperature of from about −20° C. to about −40° C. for a period of from about one day to about 5 years. In certain embodiments, the bispecific antibody has been stored at a temperature of from about −20° C. to about −40° C. for a period of from about one week to about 5 years, or from about one month to about 5 years. In certain embodiments, the bispecific antibody has been stored at a temperature of from about −20° C. to about −40° C. for a period of about one week, about two weeks, about three weeks, about four weeks, about one month, about six months, about eighteen months, about one year, about two years, about three years, about four years, or about 5 years.

As used herein, the term “about,” when used to modify a particular value or range, is understood to mean that there can be variations in the given value or range, including 20 percent, e.g., 10 percent, 5 percent, 4 percent, 3 percent, 2 percent, or 1 percent above or below of the stated value or range.

As used herein, the term “store” or “stored” refers to place or leave a bispecific antibody under certain conditions for later use or further processing. In certain embodiments, the antibody is placed under any of the temperatures described above, which can be achieved by using, e.g., freezers, refrigerated trucks, or shipping equipment that can maintain the required temperature. As can be appreciated by those of ordinary skill in the art, antibodies are frozen when stored under the storage temperatures described above.

The bispecific antibody may be stored in any suitable container that can maintain its integrity under the storage temperature. Exemplary containers include vials, bottles, bags, and carboys. Such containers are well known in the art and are available commercially. In certain embodiments, the bispecific antibody is stored in single use containers such as flexible freeze thaw containers that are commercially available, e.g., the Celsius® FFT systems. The volume of the bispecific antibody under storage is determined by the volume of the container used for the storage. In certain embodiment, the volume of the container is 5 ml, 10 mL, 100 mL, 500 mL, 1 liter (L), 2 L, 3 L, 4 L, 5 L, 6 L, 7 L, 8 L, 9 L, 10 L, or 12 L. In certain embodiments, the volume of the bispecific antibody under storage is about 5 mL, about 10 mL, about 100 mL, about 500 mL, about 1 liter (L), about 2 L, about 3 L, about 4 L, about 5 L, about 6 L, about 7 L, about 8 L, about 9 L, about 10 L, or about 12 L.

In certain embodiments, the concentration of the bispecific antibody under storage is in the range of from about 0.01 mg/mL to about 25 mg/mL, or from about 0.05 mg/mL to about 25 mg/mL, or from about 0.1 mg/mL to about 25 mg/mL, or about 0.5 mg/mL to about 25 mg/mL, or from about 1 mg/mL to about 25 mg/mL. In certain embodiments, the concentration of the bispecific antibody under storage is in the range of from about 1 mg/mL to about 20 mg/mL, or from about 1 mg/mL to about 15 mg/mL, or from about 1 mg/mL to about 10 mg/mL, or from about 1 mg/mL to about 5 mg/mL. In certain embodiments, the concentration of the bispecific antibody under storage is about 0.01 mg/mL, about 0.05 mg/mL, about 0.1 mg/mL, about 0.5 mg/mL, about 1 mg/mL, about 2 mg/mL, about 3 mg/mL, about 4 mg/mL, about 5 mg/mL, about 6 mg/mL, about 7 mg/mL, about 8 mg/mL, about 9 mg/mL, about 10 mg/mL, about 11 mg/mL, about 12 mg/mL, about 13 mg/ml, about 14 mg/mL, about 15 mg/mL, about 16 mg/mL, about 17 mg/mL, about 18 mg/mL, about 19 mg/mL, about 20 mg/mL, or about 25 mg/mL.

Also disclosed herein is a method for preparing a composition comprising a bispecific antibody. In certain embodiments, the method comprises thawing a drug substance comprising a bispecific antibody that has been stored under frozen conditions, and holding the thawed drug substance at a certain temperature for at least 4 hours.

In certain embodiments, the drug substance comprising the bispecific antibody has been stored at a temperature of from about −20° C. to about −50° C. In certain embodiments, the drug substance comprising the bispecific antibody has been stored at a temperature of from about −20° C. to about −35° C. In certain embodiments, the drug substance comprising the bispecific antibody has been stored at a temperature of from about −25° C. to about −35° C. In certain embodiments, the drug substance comprising the bispecific antibody has been stored at a temperature of about −20° C., about −30° C., about −35° C., about −40° C.′ or about −50° C. In certain embodiments, the drug substance comprising the bispecific antibody has been stored at a temperature of about −30° C.

In certain embodiments, the drug substance comprising the bispecific antibody has been stored at a temperature of from about −20° C. to about −50° C. for a period of from about one day to about 5 years. In certain embodiments, the drug substance comprising the bispecific antibody has been stored at a temperature of from about −20° C. to about −40° C. for a period of from about one week to about 5 years, or from about one month to about 5 years. In certain embodiments, the drug substance comprising the bispecific antibody has been stored at a temperature of from about −20° C. to about −40° C. for a period of about one week, about two weeks, about three weeks, about four weeks, about one month, about six months, about eighteen months, about one year, about two years, about three years, about four years, or about 5 years.

In certain embodiments, the method for preparing a composition comprising a bispecific antibody comprises holding a thawed drug substance comprising a bispecific antibody at a certain temperature for at least 4 hours wherein the drug substance has been frozen at a temperature that is at or above the glass transition temperature (Tg′) of the drug substance before thaw. In certain embodiments, the drug substance comprising the bispecific antibody has been frozen at a temperature that is at a temperature of from about −10° C. to at or above the Tg′ of the drug substance. In certain embodiments, the drug substance comprising the bispecific antibody has been frozen at a temperature of from about −20° C. to at or above the Tg′ of the drug substance. In certain embodiments, the drug substance comprising the bispecific antibody has been frozen at a temperature that is at or above the Tg′ of the drug substance (e.g., from about −10° C. to at or above the Tg′) for a period of from about one day to about 5 years or from about one week to about 5 years. In certain embodiments, the drug substance comprising the bispecific antibody has been frozen at a temperature that is at or above the Tg′ of the drug substance (e.g., from about −10° C. to at or above the Tg′) for a period of about one month, about six months, about one year, about eighteen months, about 2 years, about three years, about 4 years or about 5 years. In certain embodiments, the Tg′ of the drug substance is about −30° C., or about −32° C., or about −35° C. In certain embodiments, the drug substance comprising the bispecific antibody has been frozen at a temperature that is about −32° C. for a period of about one month, about six months, about one year, about eighteen months, about 2 years, about three years, about 4 years or about 5 years.

As can be appreciated by those of ordinary skill in the art, the glass transition temperature of a bispecific antibody or a drug substance comprising a bispecific antibody may be determined by methods known in the art, e.g., differential scanning calorimetry (DSC), thermal mechanical analysis (TMA), dynamic mechanical analysis (DMA) and dilatometry.

The drug substance comprising the bispecific antibody may have been stored or may have been frozen in any suitable container that can maintain its integrity under the storage/frozen temperature. Exemplary containers include vials, bottles, bags, and carboys. Such containers are well known in the art and are available commercially. In certain embodiments, the drug substance comprising the bispecific antibody is stored in single use containers such as flexible freeze thaw containers that are commercially available. e.g., the Celsius® FFT systems. The volume of the drug substance comprising the bispecific antibody under storage is determined by the volume of the container used for the storage. In certain embodiment, the volume of the container is 1 L, 2 L, 3 L, 4 L, 5 L, 6 L, 7 L, 8 L, 9 L, 10 L, or 12 L.

The drug substance comprising the bispecific antibody can be thawed by exposing to elevated temperatures. In certain embodiments, the drug substance is thawed at a temperature of from about 0° C. to about 50° C., or from about 0° C. to about 40° C., or from about 0° C. to about 30° C. or from about 5° C. to about 45° C. or from about 5° C. to about 30° C., or from about 10° C. to about 30° C. or from about 15° C. to about 30° C., or from about 20° C. to about 30° C., or from about 25° C. to about 30° C. In certain embodiments, the drug substance is thawed at a temperature of from about 0° C. to about 25° C., or from about 5° C. to about 25° C. or from about 10° C. to about 25° C. or from about 15° C. to about 25° C., or from about 20° C. to about 25° C. In certain embodiments, the drug substance is thawed at a temperature of about 0° C., about 5° C., about 10° C., about 15° C., about 20° C., about 25° C., about 27° C., or about 30° C., or about 40° C., or about 45° C., or about 50° C.

In certain embodiments, the method comprises holding the thawed drug substance at a temperature of from about 0° C. to about 50° C., or from about 0° C. to about 40° C., or from about 5° C. to about 50° C., or from about 5° C. to about 45° C., or from about 10° C. to about 50° C., or from about 10° C. to about 45° C., or from about 10° C. to about 30° C., or from about 15° C. to about 40° C., or from about 15° C. to about 30° C. In certain embodiment, the thawed drug substance is held at a temperature of from about 15° C. to about 25° C. In certain embodiments, the thawed drug substance is held at a temperature of about 5° C., or about 10° C., or about 15° C., or about 17° C., or about 19° C., or about 21° C. or about 23° C., or about 25° C., or about 27° C., or about 29° C., or about 40° C. or about 45° C. In certain embodiments, the thawed drug substance is held at any one of the above temperatures for a period of from about 4 hours to about 150 hours, or for a period of from about 4 hours to about 100 hours, or for a period of from about 4 hours to about 72 hours, or for a period of from about 4 hours to about 48 hours, or for a period of from about 4 hours to about 24 hours, or for a period of from about 10 hours to about 150 hours, or for a period of from about 10 hours to about 100 hours, or for a period of from about 10 hours to about 72 hours, or for a period of from about 10 hours to about 48 hours, or for a period of from about 10 hours to about 24 hours, or for a period of from about 24 hours to about 150 hours, or for a period of from about 24 hours to about 120 hours, or for a period of from about 24 hours to about 100 hours, or for a period of from about 24 hours to about 96 hours, or for a period of from about 24 hours to about 72 hours, or for a period of from about 24 hours to about 48 hours, for a period of from about 48 hours to about 100 hours, or for a period of from about 48 hours to about 96 hours, or for a period of from about 48 hours to about 72 hours, or for a period of from about 72 hours to about 100 hours, or for a period of from about 72 hours to about 96 hours.

In certain embodiments, the thawed drug substance comprising the bispecific antibody is held at a temperature of from about 5° C. to about 45° C. for at least about 4 hours. In certain embodiments, the thawed drug substance comprising the bispecific antibody is held at a temperature of from about 5° C. to about 45° C. for a period of from about 4 hours to about 120 hours, or for a period of from about 4 hours to about 96 hours, or for a period of from about 4 hours to about 72 hours, or for a period of from about 4 hours to about 48 hours, or for a period of from about 4 hours to about 24 hours. In certain embodiments, the thawed drug substance comprising the bispecific antibody is held at a temperature of from about 10° C. to about 45° C. for a period of from about 4 hours to about 100 hours, or for a period of from about 4 hours to about 50 hours. In certain embodiments, the thawed drug substance comprising the bispecific antibody is held at a temperature of about 15° C. to about 40° C. for a period of from about 4 hours to about 150 hours, or for a period of from about 4 hours to about 120 hours, or for a period of from about 4 hours to about 96 hours, or for a period of from about 4 hours to about 72 hours, or for a period of from about 4 hours to about 48 hours, or for a period of from about 4 hours to about 24 hours, or for a period of from about 10 hours to about 120 hours, or for a period of from about 10 hours to about 96 hours, or for a period of from about 10 hours to about 72 hours, or for a period of from about 10 hours to about 48 hours, or for a period of from about 10 hours to about 24 hours. In certain embodiments, the thawed drug substance comprising the bispecific antibody is held at a temperature of from about 15° C. to about 30° C. for a period of from about 10 hours to about 96 hours, or for a period of from about 10 hours to about 72 hours, or for a period of from about 10 hours to about 48 hours, or for a period of from about 10 hours to about 24 hours, or for a period of from about 24 hours to about 96 hours, or for a period of from about 24 hours to about 72 hours, or for a period of from about 24 hours to about 48 hours, or for a period of from about 48 hours to about 100 hours, or for a period of from about 48 hours to about 96 hours, or for a period of from about 48 hours to about 72 hours, or for a period of from about 72 hours to about 100 hours, or for a period of from about 72 hours to about 96 hours. In certain embodiments, the thawed drug substance comprising the bispecific antibody is held at a temperature of from about 15° C. to about 25° C. for a period of from about 15 hours to about 96 hours, or for a period of from about 15 hours to about 72 hours, or for a period of from about 15 hours to about 48 hours, or for a period of from about 24 hours to about 96 hours, or for a period of from about 24 hours to about 72 hours, or for a period of from about 24 hours to about 48 hours, or for a period of from about 48 hours to about 100 hours, or for a period of from about 48 hours to about 96 hours, or for a period of from about 48 hours to about 72 hours, or for a period of from about 72 hours to about 100 hours, or for a period of from about 72 hours to about 96 hours. In one embodiment, the thawed drug substance comprising the bispecific antibody is held at a temperature of from about 10° C. to about 30° C. for a period of from about 10 hours to about 96 hours, or for a period of from about 10 hours to about 72 hours, or for a period of from about 10 hours to about 48 hours, or for a period of from about 10 hours to about 24 hours, or for a period of from about 24 hours to about 96 hours, or for a period of from about 24 hours to about 72 hours, or for a period of from about 24 hours to about 48 hours, or for a period of from about 48 hours to about 96 hours, or for a period of from about 72 hours to about 96 hours. In another embodiment, the thawed drug substance comprising the bispecific antibody is held at a temperature of from about 10° C. to about 30° C. for a period of about 4 hours, or about 10 hours, or about 24 hours, or about 48 hours, or about 60 hours, or about 72 hours, or about 96 hours, or about 120 hours, or about 150 hours.

In certain embodiments, the thawed drug substance comprising the bispecific antibody is held at a temperature of from about 5° C. to about 45° C. for a period of time such that the level of aggregates in the drug substance comprising the bispecific antibody decreases to the about same level before storage under frozen conditions. In certain embodiments, the thawed drug substance comprising the bispecific antibody is held at a temperature of from about 5° C. to about 45° C. for a period of from about 4 hours up to a time that the level of aggregates in the drug substance comprising the bispecific antibody decreases to the about same level before storage under frozen conditions. As can be appreciated by one of ordinary skill in the art, the time required for the level of aggregates in the thawed drug substance to decrease to about the same level before storage under frozen conditions under a particular temperature can be determined by measuring the level of aggregates in the drug substance before storage under frozen conditions and at various time points after thaw using methods known and used in the art (SE-UHPLC for example), see e.g. Example 5.

In certain embodiments, the drug substance is held at the same temperature as the temperature at which the drug substance is thawed. For instance, the drug substance comprising a bispecific antibody may be thawed at a temperature, e.g., any of the thaw and holding temperatures disclosed above such as a temperature of from about 5° C. to about 45° C., and then held at the same temperature for at least 4 hours after thaw (e.g., for a period of from about 4 hours to about 150 hours, or from about 4 hours to about 120 hours, or from about 4 hours to about 96 hours, or from about 4 hours to about 72 hours, or from about 4 hours to about 48 hours, or from about 4 hours to about 24 hours, or from about 8 hours to about 96 hours, or from about 8 hours to about 72 hours, or from about 8 hours to about 48 hours, or from about 24 hours to about 72 hours, or from about 24 hours to about 48 hours). Whether or not a drug substance is thawed can be readily determined by a person of ordinary in the art.

In certain embodiments, the drug substance is held at the same temperature as the temperature at which the drug substance is thawed, and the drug substance stays under the same temperature for a total period of time (time for thaw and hold) until the level of aggregates in the drug substance comprising the bispecific antibody decreases to the about same level before storage under frozen conditions. As shown in the examples, a person of ordinary skill in the art can determine the level of aggregate in the drug substance before frozen and at various time points after thaw using methods known in the art, e.g., SE-UHPLC. In one embodiment, the drug substance is thawed and held at the same temperature of from about 5° C. to about 45° C. for a total period of from about 30 hours to about 100 hours, or for a total period of from about 30 hours to about 90 hours, or for a total period of from about 30 hours to about 80 hours, or for a total period of from about 30 hours to about 70 hours, or for a total period of from about 30 hours to about 60 hours, or for a total period of from about 30 hours to about 50 hours. In one embodiment, the drug substance is thawed and held at the same temperature of from about 15° C. to about 30° C. for a total period of from about 30 hours to about 100 hours, or for a total period of from about 30 hours to about 90 hours, or for a total period of from about 30 hours to about 80 hours, or for a total period of from about 30 hours to about 70 hours, or for a total period of from about 30 hours to about 60 hours, or for a total period of from about 30 hours to about 50 hours.

In certain embodiments, the drug substance comprising the bispecific antibody is mixed gently during thaw. Gentle mixing may be achieved by, for example, using a tilting shaking or gently inverting the container of the drug substance. In certain embodiments, the drug substance comprising the bispecific antibody is not mixed during thaw, instead the drug substance is mixed gently after thaw.

In certain embodiments, the drug substance comprising the bispecific antibody at a concentration of from about 0.01 mg/mL to about 25 mg/mL, or from about 0.05 mg/mL to about 25 mg/mL, about 0.1 mg/mL to about 25 mg/mL, about 0.5 mg/mL to about 25 mg/mL, or from about 1 mg/mL to about 25 mg/mL. In certain embodiments, the drug substance comprising the bispecific antibody at a concentration of from about 1 mg/mL to about 20 mg/mL, or from about 1 mg/mL to about 15 mg/mL, or from about 1 mg/mL to about 10 mg/mL, or from about 1 mg/mL to about 5 mg/mL. In certain embodiments, the drug substance comprising the bispecific antibody at a concentration of about 0.01 mg/mL, about 0.05 mg/mL, about 0.1 mg/mL, about 0.5 mg/mL, about 1 mg/mL, about 2 mg/mL, about 3 mg/mL, about 4 mg/mL, about 5 mg/mL, about 6 mg/mL, about 7 mg/mL, about 8 mg/ml, about 9 mg/ml, about 10 mg/mL, about 11 mg/mL, about 12 mg/ml . . . about 13 mg/mL, about 14 mg/mL, about 15 mg/mL, about 16 mg/mL, about 17 mg/mL, about 18 mg/ml, about 19 mg/mL, about 20 mg/mL, or about 25 mg/mL.

In certain embodiments, the drug substance comprising the bispecific antibody has a pH in the range of from about pH 3.5 to about pH 7.5 or from about pH 4.0 to about pH 7.0. In certain embodiments, the drug substance comprising the bispecific antibody has a pH in the range of from about pH 4.0 to about pH 6.5. In certain embodiments, the drug substance comprising the bispecific antibody has a pH in the range of from about pH 4.0 to about pH 4.8. In certain embodiments, the drug substance comprising the bispecific antibody has a pH of about 3.5, about 4.0, about 4.2, about 4.4, about 4.6, about 4.8, about 5.0, about 5.2, about 5.4, about 5.6, about 5.8, about 6, about 6.2, about 6.4, about 6.6, about 7.0, or about 7.5.

In certain embodiments, the method further comprises filtering the drug substance. In certain embodiments, the filtering step comprises sterile filtration. Sterile filtration is well known and commonly used in the art. For example, sterile filtration can be carried out using Normal Flow Filtration (NFF) where the direction of the fluid stream is perpendicular to the filter medium (e.g., a membrane) and purified liquid passes through the filter medium. In certain embodiments, agents that can reduce aggregation of the bispecific antibody. e.g., amino acids, polyols such as benzyl alcohol, cyclodextrans, dextrans and polyethylene glycol (PEG) may be added to the drug substance.

In certain embodiments, the composition prepared by the method is a pharmaceutical composition. As used herein, the term “pharmaceutical composition” is understood to refer to a formulation comprising a bispecific antibody suitable for injection and/or administration into a patient (e.g., a human) in need thereof. More particularly, a pharmaceutical composition is substantially sterile and does not contain any agents that are unduly toxic or infectious to the recipient.

In certain embodiments, the method for preparing a composition comprising a bispecific antibody further comprising aliquoting the composition into a drug product form. Such drug product forms may be presented in unit dosage form, e.g., in ampoules, single-dose containers or in multi-dose containers. The drug product forms may, if desired, be presented in a vial, pack or dispenser device which may contain one or more unit dosage forms containing the bispecific antibody.

In certain embodiments, the method for preparing a composition comprising a bispecific antibody further comprising lyophilizing the composition. In certain embodiments, the lyophilizing step is performed after aliquoting the composition into a drug product form. Methods for lyophilizing pharmaceutical compositions are well known and commonly used in the art. See e.g., Cryopreservation and Freeze-Drying Protocols (J. G. Day and G. N. Stacey ed., Springer 2017). The lyophilizing step may be performed before or after the aliquoting step.

In certain embodiments, the method for preparing a composition comprising a bispecific antibody further comprising spray drying the composition. Methods for spray drying pharmaceutical compositions are well known and commonly used in the art. E.g., Niven, R., Prestrelski, S. J., Treuheit, M. J., Ip. A. Y. and Arakawa, T. Protein Nebulization II. Stabilization of G-CSF to air-jet nebulization and the role of protectants. (1996) Int. J. Pharm. 127, 191-20. The spray drying step may be performed before or after the aliquoting step.

Methods disclosed herein reduce aggregates of bispecific antibodies. The methods are based on the surprising finding that aggregates formed during storage under frozen conditions (e.g., about −20° C. to about −40° C.) is reduced when the thawed antibodies are held at a certain temperature, e.g., from about 5° C. to about 45° C., for a period of at least 4 hours (e.g., for a period of from about 4 hours to about 96 hours). Not wishing to be bound by any theory, it is believed that the aggregates reverse back to the unaggregated state after the holding period.

In certain embodiments, the aggregates comprise HMW aggregates. In certain embodiments, the bispecific antibody comprises less than about 5%, or less than about 3%, or less than about 2%, or less than about 1%, or less than about 0.5% of the HMW aggregates after the holding period. In certain embodiments, the HMW aggregates formed under frozen conditions is reduced to the same level or substantially the same level as before freezing. In certain embodiments, the HMW aggregates comprise dimers of the bispecific antibody. In certain embodiments, the bispecific antibody comprises less than about 19% of dimers of the bispecific antibody after the holding period. In certain embodiments, the bispecific antibody comprises less than about 0.5% of dimers of the bispecific antibody after the holding period.

In certain embodiments, the drug substance comprising the bispecific antibody comprises less than about 5%, or less than about 3%, or less than about 2%, or less than about 1%, or less than about 0.5% of HMW aggregates after the holding period. In certain embodiments, the HMW aggregates comprise dimers of the bispecific antibody. In certain embodiments, the drug substance comprising the bispecific antibody comprises less than about 1% of dimers of the bispecific antibody after the holding period. In certain embodiments, the drug substance comprising the bispecific antibody comprises less than about 0.5% of dimers of the bispecific antibody after the holding period.

Methods disclosed herein have no or substantially no impact on stability attributes of bispecific antibodies or drug substance comprising bispecific antibodies. In certain embodiments, the methods produce a bispecific antibody or a drug substance comprising a bispecific antibody having the same or substantially the same color and/or clarity compared to the bispecific antibody or the drug substance before freezing. In certain embodiments, the methods produce a bispecific antibody or a drug substance comprising a bispecific antibody having the same or substantially the same charge variants compared to the bispecific antibody or the drug substance before freezing. In certain embodiments, the methods produce a bispecific antibody or a drug substance comprising a bispecific antibody having the same or substantially the same potency compared to the bispecific antibody or the drug substance before freezing. In certain embodiments, the methods produce a bispecific antibody or a drug substance comprising a bispecific antibody having the same or substantially the same level of clipping compared to the bispecific antibody or the drug substance before freezing. In certain embodiments, the methods produce a bispecific antibody or a drug substance comprising a bispecific antibody having the same or substantially the same chemical modifications (e.g., glycosylation) compared to the bispecific antibody or the drug substance before freezing. In certain embodiments, the methods produce a bispecific antibody or a drug substance comprising a bispecific antibody having the same or substantially the same pH compared to the bispecific antibody of the drug substance before freezing.

### Bispecific Antibodies

Bispecific antibodies that may be used in the methods disclosed herein include those that tend to aggregate, e.g., due to hydrophobic interactions between different regions of the antibody, under frozen conditions, e.g., at temperatures in the range of −20° C. to −50° C. As used herein, the term “bispecific antibodies” is understood to refer to antibodies capable of specifically binding to two different antigens or targets or epitopes. In certain embodiments, the bispecific antibody comprises a first domain specifically binds to one antigen or target and a second domain specifically binds to another antigen or target. In certain embodiments, the first domain of the bispecific antibody specifically binds to a target cell surface antigen and the second binding domain of the bispecific antibody specifically binds to human CD3, a subunit of the T cell receptor complex on T cells. In certain preferred embodiments, the bispecific antibody is a bispecific T cell engager (BiTE) antibody construct. See e.g., WO2008119567 and WO2017134140.

As under herein, the term “domain specifically binds” or “domain that binds” is understood to refer to a domain that specifically binds to/interacts with/recognizes a given target or epitope. The binding domain of an antibody construct comprises the minimum structural requirements of an antibody which allow for the target binding. This minimum requirement may be defined by the presence of at least the three light chain CDRs (i.e. CDR1, CDR2 and CDR3 of the light chain variable region (VL) region) and/or the three heavy chain CDRs (i.e. CDR1, CDR2 and CDR3 of the heavy chain variable region (VH) region), preferably of all six CDRs. Preferably, those CDRs are comprised in the framework of an antibody VL and an antibody VH. The term includes fragments of full-length antibodies and antibody variants. Examples of antibody fragments, antibody variants or binding domains include (1) a Fab fragment, a monovalent fragment having the VL, VH, CL and CH1 domains; (2) a F(ab′)₂ fragment, a bivalent fragment having two Fab fragments linked by a disulfide bridge at the hinge region; (3) an Fd fragment having the two VH and CH1 domains; (4) an Fv fragment having the VL and VH domains of a single arm of an antibody, (5) a dAb fragment (Ward et al., (1989) Nature 341:544-546), which has a VH domain; (6) an isolated complementarity determining region (CDR), and (7) a single chain Fv (scFv), the latter being preferred (for example, derived from an scFV-library). Additional antibody fragments include VH, VHH, VL, (s) dAb, Fab′, and “r IgG” (“half antibody”).

The term “antibody construct” is understood to refer to a molecule in which the structure and/or function is/are based on the structure and/or function of an antibody, e.g., of a full-length or whole immunoglobulin molecule and/or is/are drawn from the variable heavy chain (VH) and/or variable light chain (VL) domains of an antibody or fragment thereof. An antibody construct is hence capable of binding to its specific target or antigen. Antibody construct also includes modified fragments of antibodies, also called antibody variants, such as scFv, di-scFv or bi(s)-scFv, scFv-Fc, scFv-zipper, scFab, Fab₂, Fab₃, diabodies, single chain diabodies, tandem diabodies (Tandab's), tandem di-scFv, tandem tri-scFv, “multibodies” such as triabodies or tetrabodies, and single domain antibodies such as nanobodies or single variable domain antibodies comprising merely one variable domain, which might be VHH, VH or VL, that specifically bind an antigen or epitope independently of other V regions or domains.

As used herein, the terms “single-chain Fv,” “single-chain antibodies” or “scFv” are understood to refer to single polypeptide chain antibody fragments that comprise the variable regions from both the heavy and light chains, but lack the constant regions. Generally, a single-chain antibody further comprises a polypeptide linker between the VH and VL domains which enables it to form the desired structure which would allow for antigen binding. Single chain antibodies are discussed in detail by Pluckthun in The Pharmacology of Monoclonal Antibodies, vol. 113, Rosenburg and Moore eds. Springer-Verlag, New York, pp. 269-315 (1994). Various methods of generating single chain antibodies are known, including those described in U.S. Pat. Nos. 4,694,778 and 5,260,203; International Patent Application Publication No. WO 88/01649; Bird (1988) Science 242:423-442; Huston et al. (1988) Proc. Natl. Acad. Sci. USA 85:5879-5883; Ward et al. (1989) Nature 334:54454; Skerra et al. (1988) Science 242:1038-1041. In specific embodiments, single-chain antibodies can also be bispecific human, and/or humanized and/or synthetic.

In certain embodiments, the first and the second domain of the bispecific antibody is a “bispecific single chain antibody construct”, more preferably a bispecific “single chain Fv” (scFv). Although the two domains of the Fv fragment, VL and VH, are coded for by separate genes, they can be joined, using recombinant methods, by a synthetic linker—as described herein—that enables them to be made as a single protein chain in which the VL and VH regions pair to form a monovalent molecule; see e.g., Huston et al. (1988) Proc. Natl. Acad. Sci USA 85:5879-5883). These antibody fragments are obtained using conventional techniques known to those with skill in the art, and the fragments are evaluated for function in the same manner as are whole or full-length antibodies. A scFv is hence a fusion protein of the variable region of the heavy chain (VH) and of the light chain (VL) of immunoglobulins, usually connected with a short linker peptide of about ten to about 25 amino acids, preferably about 15 to 20 amino acids. The linker is usually rich in glycine for flexibility, as well as serine or threonine for solubility, and can either connect the N-terminus of the VH with the C-terminus of the VL, or vice versa. The scFv retains the specificity of the original immunoglobulin, despite removal of the constant regions and introduction of the linker.

Bispecific single-chain variable fragments (bi-scFvs or di-scFvs having the format (scFv) 2 can be engineered by linking two scFv molecules (e.g. with linkers as described herein). The linking can be done by producing a single peptide chain with two VH regions and two VL regions, yielding tandem scFvs (see e.g. Kufer P. et al., (2004) Trends in Biotechnology 22 (5): 238-244). Another possibility is the creation of scFv molecules with linker peptides that are too short for the two variable regions to fold together (e.g. about five amino acids), forcing the scFvs to dimerize. This type is known as diabodies (see e.g. Hollinger, Philipp et al., (July 1993) Proceedings of the National Academy of Sciences of the United States of America 90 (14): 6444-8).

In certain embodiments, the first and the second domain of the bispecific antibody specifically binds to a target cell surface antigen and to human CD3, respectively. In certain embodiments, the first and second domain of the bispecific antibody form a bispecific antibody construct in a format selected from the group consisting of (scFv) 2, scFv-single domain mAb, diabody and oligomers of any of those formats.

In certain embodiments, either the first, the second or the first and the second domain may comprise a single domain antibody, respectively the variable domain or at least the CDRs of a single domain antibody. Single domain antibodies comprise merely one (monomeric) antibody variable domain which is able to bind selectively to a specific antigen, independently of other V regions or domains. The first single domain antibodies were engineered from heavy chain antibodies found in camelids, and these are called VHH fragments. Cartilaginous fishes also have heavy chain antibodies (IgNAR) from which single domain antibodies called V_{NAR} fragments can be obtained. An alternative approach is to split the dimeric variable domains from common immunoglobulins e.g. from humans or rodents into monomers, hence obtaining VH or VL as a single domain Ab. Although most research into single domain antibodies is currently based on heavy chain variable domains, nanobodies derived from light chains have also been shown to bind specifically to target epitopes. Examples of single domain antibodies are called sdAb, nanobodies or single variable domain antibodies.

In certain embodiments, the first (binding) domain of the bispecific antibody binds to a target cell surface antigen. In some embodiment, the target cell surface antigen is CD70. CD70 (also known as CD27L or TNFSF7) is a type II integral membrane protein whose normal expression is restricted to a subset of activated T and B cells, mature dendritic cells and thymic medullar epithelial cells.

In other embodiments, the target cell surface antigen is a tumor antigen. The term “tumor antigen” as used herein is understood to refer to those antigens that are presented on tumor cells. These antigens can be presented on the cell surface with an extracellular part, which is often combined with a transmembrane and cytoplasmic part of the molecule. These antigens can sometimes be presented only by tumor cells and not by the normal ones. Tumor antigens can be exclusively expressed on tumor cells or might represent a tumor specific mutation compared to normal cells. In this case, they are called tumor-specific antigens. More common are antigens that are presented by tumor cells and normal cells, and they are called tumor-associated antigens. These tumor-associated antigens can be overexpressed compared to normal cells or are accessible for antibody binding in tumor cells due to the less compact structure of the tumor tissue compared to normal tissue. In some embodiments, the second (binding) domain binds to tumor antigens selected from CD19, CD33, epidermal growth factor receptor variant iii (EGFRvIII), mesothelin (MSLN), cadherin 19 (CDH19), FMS-like tyrosine kinase 3 (FLT3), delta-like ligand 3 (DLL3), Placental-Cadherin (CDH3), B-cell maturation antigen (BCMA) or prostate-specific membrane antigen (PSMA). In some embodiments, the tumor antigens are human tumor antigens.

In certain embodiments, the second (binding) domain of the bispecific antibody binds to human CD3 epsilon on the surface of a T cell. In certain preferred embodiments, the second domain of the bispecific antibody binds to an extracellular epitope of the human CD38 chain. In some preferred embodiments, the second domain of the bispecific antibody that binds to an extracellular epitope of the human CD3 comprises a VL region comprising CDR-L1, CDR-L2 and CDR-L3 selected from:
(a) CDR-L1 as depicted in SEQ ID NO: 27 of WO 2008/119567, CDR-L2 as depicted in SEQ ID NO: 28 of WO 2008/119567 and CDR-L3 as depicted in SEQ ID NO: 29 of WO 2008/119567;
(b) CDR-L1 as depicted in SEQ ID NO: 117 of WO 2008/119567, CDR-L2 as depicted in SEQ ID NO: 118 of WO 2008/119567 and CDR-L3 as depicted in SEQ ID NO: 119 of WO 2008/119567; and
(c) CDR-L1 as depicted in SEQ ID NO: 153 of WO 2008/119567, CDR-L2 as depicted in SEQ ID NO: 154 of WO 2008/119567 and CDR-L3 as depicted in SEQ ID NO: 155 of WO 2008/119567.

In another preferred embodiment, the second domain of the bispecific antibody binds to an extracellular epitope of the human CD3 epsilon chain and comprises a VH region comprising CDR-H1, CDR-H2 and CDR-H3 selected from:
(a) CDR-H1 as depicted in SEQ ID NO: 12 of WO 2008/119567, CDR-H2 as depicted in SEQ ID NO: 13 of WO 2008/119567 and CDR-H3 as depicted in SEQ ID NO: 14 of WO 2008/119567;
(b) CDR-H1 as depicted in SEQ ID NO: 30 of WO 2008/119567, CDR-H2 as depicted in SEQ ID NO: 31 of WO 2008/119567 and CDR-H3 as depicted in SEQ ID NO: 32 of WO 2008/119567;
(c) CDR-H1 as depicted in SEQ ID NO: 48 of WO 2008/119567, CDR-H2 as depicted in SEQ ID NO: 49 of WO 2008/119567 and CDR-H3 as depicted in SEQ ID NO: 50 of WO 2008/119567;
(d) CDR-H1 as depicted in SEQ ID NO: 66 of WO 2008/119567, CDR-H2 as depicted in SEQ ID NO: 67 of WO 2008/119567 and CDR-H3 as depicted in SEQ ID NO: 68 of WO 2008/119567;
(e) CDR-H1 as depicted in SEQ ID NO: 84 of WO 2008/119567, CDR-H2 as depicted in SEQ ID NO: 85 of WO 2008/119567 and CDR-H3 as depicted in SEQ ID NO: 86 of WO 2008/119567;
(f) CDR-H1 as depicted in SEQ ID NO: 102 of WO 2008/119567, CDR-H2 as depicted in SEQ ID NO: 103 of WO 2008/119567 and CDR-H3 as depicted in SEQ ID NO: 104 of WO 2008/119567;
(g) CDR-H1 as depicted in SEQ ID NO: 120 of WO 2008/119567, CDR-H2 as depicted in SEQ ID NO: 121 of WO 2008/119567 and CDR-H3 as depicted in SEQ ID NO: 122 of WO 2008/119567;
(h) CDR-H1 as depicted in SEQ ID NO: 138 of WO 2008/119567, CDR-H2 as depicted in SEQ ID NO: 139 of WO 2008/119567 and CDR-H3 as depicted in SEQ ID NO: 140 of WO 2008/119567;
(i) CDR-H1 as depicted in SEQ ID NO: 156 of WO 2008/119567, CDR-H2 as depicted in SEQ ID NO: 157 of WO 2008/119567 and CDR-H3 as depicted in SEQ ID NO: 158 of WO 2008/119567; and
(j) CDR-H1 as depicted in SEQ ID NO: 174 of WO 2008/119567, CDR-H2 as depicted in SEQ ID NO: 175 of WO 2008/119567 and CDR-H3 as depicted in SEQ ID NO: 176 of WO 2008/119567.

In certain preferred embodiments, the above described three groups of VL CDRs are combined with the above described ten groups of VH CDRs within the second binding domain to form (30) groups, each comprising CDR 1-3.

It is also preferred that the second domain that binds to CD3 comprises a VH region selected from the group of VH regions as depicted in SEQ ID NO: 15, 19, 33, 37, 51, 55, 69, 73, 87, 91, 105, 109, 123, 127, 141, 145, 159, 163, 177 or 181 of WO 2008/119567 or as depicted in the present sequence listing as SEQ ID NO: 15 or 24.

It is preferred that the second domain which binds to CD3 comprises a VL region selected from the group of VL regions as depicted in SEQ ID NO: 17, 21, 35, 39, 53, 57, 71, 75, 89, 93, 107, 111, 125, 129, 143, 147, 161, 165, 179 or 183 of WO 2008/119567 or as depicted in the present sequence listing as SEQ ID NO: 16 or 25.

More preferably, the bispecific antibody is characterized by a second domain which binds to CD3 comprising a VL region and a VH region selected from:
(a) a VL region as depicted in SEQ ID NO: 17 or 21 of WO 2008/119567 and a VH region as depicted in SEQ ID NO: 15 or 19 of WO 2008/119567;
(b) a VL region as depicted in SEQ ID NO: 35 or 39 of WO 2008/119567 and a VH region as depicted in SEQ ID NO: 33 or 37 of WO 2008/119567;
(c) a VL region as depicted in SEQ ID NO: 53 or 57 of WO 2008/119567 and a VH region as depicted in SEQ ID NO: 51 or 55 of WO 2008/119567;
(d) a VL region as depicted in SEQ ID NO: 71 or 75 of WO 2008/119567 and a VH region as depicted in SEQ ID NO: 69 or 73 of WO 2008/119567;
(e) a VL region as depicted in SEQ ID NO: 89 or 93 of WO 2008/119567 and a VH region as depicted in SEQ ID NO: 87 or 91 of WO 2008/119567;
(f) a VL region as depicted in SEQ ID NO: 107 or 111 of WO 2008/119567 and a VH region as depicted in SEQ ID NO: 105 or 109 of WO 2008/119567;
(g) a VL region as depicted in SEQ ID NO: 125 or 129 of WO 2008/119567 and a VH region as depicted in SEQ ID NO: 123 or 127 of WO 2008/119567;
(h) a VL region as depicted in SEQ ID NO: 143 or 147 of WO 2008/119567 and a VH region as depicted in SEQ ID NO: 141 or 145 of WO 2008/119567;
(i) a VL region as depicted in SEQ ID NO: 161 or 165 of WO 2008/119567 and a VH region as depicted in SEQ ID NO: 159 or 163 of WO 2008/119567; or
(j) a VL region as depicted in SEQ ID NO: 179 or 183 of WO 2008/119567 and a VH region as depicted in SEQ ID NO: 177 or 181 of WO 2008/119567.

Also in a preferred embodiment, the bispecific antibody comprises a second domain which binds to CD3 comprising a VL region as depicted in SEQ ID NO: 16 or 25 and a VH region as depicted in the present sequence listing as SEQ ID NO: 15 or 24.

A preferred embodiment of the above described bispecific antibody is characterized by the second domain which binds to CD3 comprising an amino acid sequence selected from SEQ ID NOs: 23, 25, 41, 43, 59, 61, 77, 79, 95, 97, 113, 115, 131, 133, 149, 151, 167, 169, 185 or 187 of WO 2008/119567 or depicted in the present sequence listing as SEQ ID NO: 26.

According to a preferred embodiment, the first and/or the second domain have the following format: The pairs of VH regions and VL regions are in the format of a single chain antibody (scFv). The VH and VL regions are arranged in the order VH-VL or VL-VH. It is preferred that the VH-region is positioned N-terminally of a linker sequence, and the VL-region is positioned C-terminally of the linker sequence. In certain embodiments, the first and second domain of the bispecific antibody form a bispecific antibody in a format selected from (scFv) 2, scFv-single domain mAb, diabody or oligomers of any of those formats.

In certain preferred embodiments, the bispecific antibody further comprises a third domain. In certain embodiments, the third domain is a single-chain Fc (scFc) domain. In certain preferred embodiments, the scFc domain is a scFc half-life extended (HLE) domain.

The term “Fc” portion or “Fc” monomer is understood to refer to a polypeptide comprising at least one domain having the function of a CH2 domain and at least one domain having the function of a CH3 domain of an immunoglobulin molecule. The polypeptide comprising those CH domains is a “polypeptide monomer”. An Fc monomer can be a polypeptide comprising at least a fragment of the constant region of an immunoglobulin excluding the first constant region immunoglobulin domain of the heavy chain (CH1), but maintaining at least a functional part of one CH2 domain and a functional part of one CH3 domain, wherein the CH2 domain is amino terminal to the CH3 domain. In a preferred embodiment, an Fc monomer can be a polypeptide constant region comprising a portion of the Ig-Fc hinge region, a CH2 region and a CH3 region, wherein the hinge region is amino terminal to the CH2 domain. It is believed that the hinge region of the bispecific antibody promotes dimerization. Such Fc polypeptide molecules can be obtained by, e.g., papain digestion of an immunoglobulin region (of course resulting in a dimer of two Fc polypeptide). In another embodiment, an Fc monomer can be a polypeptide region comprising a portion of a CH2 region and a CH3 region. Such Fc polypeptide molecules can be obtained by, e.g., pepsin digestion of an immunoglobulin molecule. In one embodiment, the polypeptide sequence of an Fc monomer is substantially similar to an Fc polypeptide sequence of: an IgG1 Fc region, an IgG2 Fc region, an IgG3 Fc region, an IgG4 Fc region, an IgM Fc region, an IgA Fc region, an IgD Fc region and an IgE Fc region. (See, e.g., Padlan, Molecular Immunology, 31 (3), 169-217 (1993)). In one embodiment, the Fc monomer has the amino acid sequence as disclosed in WO2014/153063. Because there is some variation between immunoglobulins, and solely for clarity, Fc monomer is understood to refer to the last two heavy chain constant region immunoglobulin domains of IgA, IgD, and IgG, and the last three heavy chain constant region immunoglobulin domains of IgE and IgM. As mentioned, the Fc monomer can also include the flexible hinge N-terminal to these domains. For IgA and IgM, the Fc monomer may include the J chain. For IgG, the Fc portion comprises immunoglobulin domains CH2 and CH3 and the hinge between the first two domains and CH2. Although the boundaries of the Fc portion may vary, an example for a human IgG heavy chain Fc portion comprising a functional hinge, CH2 and CH3 domain can be defined e.g. to comprise residues D231 (of the hinge domain-corresponding to D234 in Table 1 below)) to P476, respectively L476 (for IgG4) of the carboxyl-terminus of the CH3 domain, wherein the numbering is according to Kabat. The two Fc portions or Fc monomers, which are fused to each other via a peptide linker define the third domain of the antibody construct of the invention, which may also be defined as scFc domain.

An IgG hinge region can be identified by analogy using the Kabat numbering as set forth in Table 1. It is envisaged that a hinge domain/region of the third domain comprises the amino acid residues corresponding to the IgG1 sequence stretch of D234 to P243 according to the Kabat numbering. It is likewise envisaged that a hinge domain/region of the third domain comprises or consists of the IgG1 hinge sequence DKTHTCPPCP (SEQ ID NO: 191) (corresponding to the stretch D234 to P243 as shown in Table 1 below-variations of the sequence are also envisaged provided that the hinge region still promotes dimerization). In a preferred embodiment, the glycosylation site at Kabat position 314 of the CH2 domains in the third domain of the antibody construct is removed by a N314X substitution, wherein X is any amino acid excluding Q. The substitution is preferably a N314G substitution. In a more preferred embodiment, said CH2 domain additionally comprises the following substitutions (position according to Kabat) V321C and R309C (these substitutions introduce the intra domain cysteine disulfide bridge at Kabat positions 309 and 321).

TABLE 1
Kabat numbering of the amino acid residues of the hinge region

| IMGT numbering | IgG₁ amino acid | Kabat |
|---|---|---|
| for the hinge | translation | numbering |
| 1 | (E) | 226 |
| 2 | P | 227 |
| 3 | K | 228 |
| 4 | S | 232 |
| 5 | C | 233 |
| 6 | D | 234 |
| 7 | K | 235 |
| 8 | T | 236 |
| 9 | H | 237 |
| 10 | T | 238 |
| 11 | C | 239 |
| 12 | P | 240 |
| 13 | P | 241 |
| 14 | C | 242 |
| 15 | P | 243 |

In some embodiments, the hinge domain/region comprises or consists of the IgG2 subtype hinge sequence ERKCCVECPPCP (SEQ ID NO: 192), the IgG3 subtype hinge sequence ELKTPLDTTHTCPRCP (SEQ ID NO: 193) or ELKTPLGDTTHTCPRCP (SEQ ID NO: 194), and/or the IgG4 subtype hinge sequence ESKYGPPCPSCP (SEQ ID NO: 195). The IgG1 subtype hinge sequence may be the following one EPKSCDKTHTCPPCP (as shown in Table 1 and SEQ ID NO: 196). These core hinge regions are thus also envisaged in the context of the bispecific antibody.

The location and sequence of the IgG CH2 and IgG CD3 domain can be identified by analogy using the Kabat numbering as set forth in Table 2:

TABLE 2
Kabat numbering of the amino acid residues
of the IgG CH2 and CH3 region

| IgG | CH2 aa | CH2 Kabat | CH3 aa | CH3 Kabat |
|---|---|---|---|---|
| subtype | translation | numbering | translation | numbering |
| IgG₁ | APE . . . KAK | 244 . . . 360 | GQP . . . PGK | 361 . . . 478 |
| IgG₂ | APP . . . KTK | 244 . . . 360 | GQP . . . PGK | 361 . . . 478 |
| IgG₃ | APE . . . KTK | 244 . . . 360 | GQP . . . PGK | 361 . . . 478 |
| IgG₄ | APE . . . KAK | 244 . . . 360 | GQP . . . LGK | 361 . . . 478 |

In one embodiment, the emphasized bold amino acid residues in the CH3 domain of the first or both Fc monomers are deleted.

In the event that a linker is used to fuse the first domain to the second domain, or the first or second domain to the third domain, the linker is preferably of a length and sequence sufficient to ensure that each of the first and second domains can, independently from one another, retain their differential binding specificities. For peptide linkers which connect the at least two binding domains (or two variable domains) in the bispecific antibody construct, those peptide linkers are preferred comprising only a few number of amino acid residues, e.g. 12 amino acid residues or less. Thus, peptide linkers of 12, 11, 10, 9, 8, 7, 6 or 5 amino acid residues are preferred. An envisaged peptide linker with less than 5 amino acids comprises 4, 3, 2 or one amino acid(s), wherein Gly-rich linkers are preferred.

A particularly preferred “single” amino acid “peptide linker” is Gly. Accordingly, the peptide linker may consist of the single amino acid Gly. In a preferred embodiment, a peptide linker is characterized by the amino acid sequence Gly-Gly-Gly-Gly-Ser, i.e. Gly4Ser (SEQ ID NO: 197), or polymers thereof, i.e. (Gly4Ser)x, where x is an integer of 1 or greater (e.g. 2 or 3). In another preferred embodiment, the peptide linker has the amino acid sequence Gly-Gly-Gly-Gly-Ser, i.e. Gly4Ser (SEQ ID NO: 197), or polymers thereof, i.e. (Gly4Ser)x, where x is an integer of 5 or greater (e.g. 5, 6, 7, 8 etc. or greater). In certain embodiments, x is 6 being preferred ((Gly4Ser)6). The characteristics of the peptide linker, which comprise the absence of the promotion of secondary structures, are known in the art and are described e.g. in Dall'Acqua et al. (Biochem. (1998) 37, 9266-9273), Cheadle et al. (Mol Immunol (1992) 29, 21-30) and Raag and Whitlow (FASEB (1995) 9 (1), 73-80). Peptide linkers do not promote any secondary structures are preferred. Methods for preparing fused and operatively linked bispecific single chain constructs and expressing them in mammalian cells or bacteria are well-known in the art (e.g. WO 99/54440 or Sambrook et al., Molecular Cloning: A Laboratory Manual, Cold Spring Harbor Laboratory Press, Cold Spring Harbor, New York, 2001).

A preferred embodiment of the peptide linker for a fusion of the first and the second domain has the amino acid sequence of Gly-Gly-Gly-Gly-Ser, i.e. Gly4Ser (SEQ ID NO: 197). A preferred linker embodiment of the peptide linker for a fusion the second and the third domain is a (Gly) 4-linker, respectively G4-linker.

The peptide linker, by which the polypeptide monomers (“Fc portion” or “Fc monomer”) of the third domain are fused to each other, preferably comprises at least 25 amino acid residues (25, 26, 27, 28, 29, 30 etc.). More preferably, this peptide linker comprises at least 30 amino acid residues (30, 31, 32, 33, 34, 35 etc.). It is also preferred that the linker comprises up to 40 amino acid residues, more preferably up to 35 amino acid residues, most preferably exactly 30 amino acid residues. A preferred embodiment of such peptide linker is characterized by the amino acid sequence Gly-Gly-Gly-Gly-Ser, i.e. Gly4Ser (SEQ ID NO: 197), or polymers thereof, i.e. (Gly4Ser)x, where x is an integer of 5 or greater (e.g. 6, 7 or 8). Preferably the integer is 6 or 7, more preferably the integer is 6.

In some preferred embodiments, the third domain of the bispecific antibody is a HLE domain with an amino to carboxyl order:
hinge-CH2-CH3-linker-hinge-CH2-CH3.

In certain embodiments, the CH2 domain of one or preferably each (both) polypeptide monomers of the third domain comprises an intra domain cysteine disulfide bridge. As known in the art the term “cysteine disulfide bridge” refers to a functional group with the general structure R—S—S—R. The linkage is also called an SS-bond or a disulfide bridge and is derived by the coupling of two thiol groups of cysteine residues. It is particularly preferred for the bispecific antibody that the cysteines forming the cysteine disulfide bridge in the mature antibody construct are introduced into the amino acid sequence of the CH2 domain corresponding to 309 and 321 (Kabat numbering).

In one embodiment, a glycosylation site in Kabat position 314 of the CH2 domain is removed. It is preferred that this removal of the glycosylation site is achieved by a N314X substitution, wherein X is any amino acid excluding Q. The substitution is preferably a N314G substitution. In a more preferred embodiment, said CH2 domain additionally comprises the following substitutions (position according to Kabat) V321C and R309C (these substitutions introduce the intra domain cysteine disulfide bridge at Kabat positions 309 and 321).

It is assumed that the preferred features of the bispecific compared e.g. to the bispecific heteroFc antibody construct known in the art may be inter alia related to the introduction of the above described modifications in the CH2 domain. Thus, it is preferred that the CH2 domains in the third domain of the bispecific antibody comprise the intra domain cysteine disulfide bridge at Kabat positions 309 and 321 and/or the glycosylation site at Kabat position 314 is removed by a N314X substitution as above, preferably by a N314G substitution.

In a further preferred embodiment, the CH2 domains in the third domain of the bispecific antibody comprise the intra domain cysteine disulfide bridge at Kabat positions 309 and 321 and the glycosylation site at Kabat position 314 is removed by a N314G substitution.

In certain embodiments, the third domain of the bispecific antibody comprises or consists in an amino to carboxyl order: DKTHTCPPCP (SEQ ID NO: 191) (i.e. hinge)-CH2-CH3-linker-DKTHTCPPCP (SEQ ID NO: 191) (i.e. hinge)-CH2-CH3. The peptide linker of the aforementioned bispecific antibody is in a preferred embodiment characterized by the amino acid sequence Gly-Gly-Gly-Gly-Ser, i.e. Gly4Ser (SEQ ID NO: 197, or polymers thereof, i.e. (Gly4Ser)x, where x is an integer of 5 or greater (e.g. 5, 6, 7, 8 etc. or greater), 6 being preferred ((Gly4Ser)6). The antibody may further comprise the aforementioned substitutions N314X, preferably N314G, and/or the further substitutions V321C and R309C.

The bispecific antibody may also comprise additional domains, which are e.g. helpful in the isolation of the molecule or relate to an adapted pharmacokinetic profile of the molecule. Domains helpful for the isolation of an antibody construct may be selected from peptide motives or secondarily introduced moieties, which can be captured in an isolation method, e.g. an isolation column. Non-limiting embodiments of such additional domains comprise peptide motives known as Myc-tag, HAT-tag, HA-tag, TAP-tag, GST-tag, chitin binding domain (CBD-tag), maltose binding protein (MBP-tag), Flag-tag, Strep-tag and variants thereof (e.g. StrepII-tag) and His-tag. All herein disclosed bispecific antibody may comprise a His-tag domain, which is generally known as a repeat of consecutive His residues in the amino acid sequence of a molecule, preferably of five, and more preferably of six His residues (hexa-histidine). The His-tag may be located e.g. at the N- or C-terminus of the antibody construct, preferably it is located at the C-terminus. Most preferably, a hexa-histidine tag (HHHHHH) (SEQ ID NO:198) is linked via peptide bond to the C-terminus of the bispecific antibody. Additionally, a conjugate system of PLGA-PEG-PLGA may be combined with a poly-histidine tag for sustained release application and improved pharmacokinetic profile.

In certain embodiments, the bispecific antibody comprises a first domain and a second domain, wherein:
(i) the first domain comprises two antibody variable domains and the second domain comprises two antibody variable domains;
(ii) the first domain comprises one antibody variable domain and the second domain comprises two antibody variable domains;
(iii) the first domain comprises two antibody variable domains and the second domain comprises one antibody variable domain; or
(iv) the first domain comprises one antibody variable domain and the second domain comprises one antibody variable domain.

Accordingly, the first and the second domain may be binding domains comprising each two antibody variable domains such as a VH and a VL domain. Examples for such binding domains comprising two antibody variable domains were described herein above and comprise e.g. Fv fragments, scFv fragments or Fab fragments described herein above. Alternatively, either one or both of those binding domains may comprise only a single variable domain. Examples for such single domain binding domains where described herein above and comprise e.g. nanobodies or single variable domain antibodies comprising merely one variable domain, which might be VHH, VH or VL, that specifically bind an antigen or epitope independently of other V regions or domains.

In some preferred embodiments, the bispecific antibody comprises a first domain, a second domain and a third domain, wherein the first domain binds to CD70 and the second domain binds to human CD3, and the third domain is a HLE domain with an amino to carboxyl order: hinge-CH2-CH3-linker-hinge-CH2-CH3. In other preferred embodiments, the bispecific antibody comprises a first domain, a second domain and a third domain, wherein the first domain binds to a tumor antigen selected from CD19, CD33, EGFRvIII, MSLN, CDH19, FLT3, DLL3, CDH3, BCMA or PSMA, and the second domain binds to human CD3, and the third domain is a HLE domain with an amino to carboxyl order: hinge-CH2-CH3-linker-hinge-CH2-CH3. In a preferred embodiment, the first and second domain are fused to the third domain via a peptide linker. Preferred peptide linker have been described herein above and are characterized by the amino acid sequence Gly-Gly-Gly-Gly-Ser, i.e. Gly4Ser, or polymers thereof, i.e. (Gly4Ser)x, where x is an integer of 1 or greater (e.g. 2, 3, 4, 5, 6, or 7).

In some embodiments, the bispecific antibody is characterized by having an amino acid sequence selected from:
(a) SEQ ID NOs: 37 to 41; CD33
(b) SEQ ID NOs: 51 and 52; EGFRvIII
(c) SEQ ID NOs: 62, 63 and 64; MSLN
(d) SEQ ID NOs: 74 to 82 CDH19
(e) SEQ ID NOs: 103 and 104 DLL3
(f) SEQ ID NOs: 17, 113 and 114 CD19
(g) SEQ ID NOs: 92 and 93 FLT3
(h) SEQ ID NOs: 124 and 125 CDH3
(i) SEQ ID NOs: 135 and 136 BCMA
(j) SEQ ID NOs: 146 to 151, 161 to 168 and 176 to 181 PSMA or
(k) SEQ ID NOs: 188 to 190 CD70

Any of the foregoing bispecific antibody may or may not be provided with a third domain, which is a half-life extended (HLE) domain, which preferably is a scFc domain or a heteroFc domain or an albumin binding domain. The second domain of the bispecific antibody which binds to human CD3 may be connected to the N-terminus or the C-terminus of the HLE domain (e.g., via a linker as described above).

In some embodiments, the bispecific antibody is a CD70×CD3 bispecific antibody, which comprises a first domain that binds to CD70 and a second domain that binds to CD3. In one embodiment, the first domain binds to CD70 and has the CDRs as depicted in SEQ ID NOs: 182 to 187, the second domain binds to CD3 and has the CDRs as depicted in SEQ ID NOs 9 to 14. In some embodiments, the bispecific antibody further comprises a HLE domain (third domain). In one embodiment, the bispecific antibody comprises a VH and a VL, wherein the VH comprising, consisting essentially of, or consisting of the amino acid sequence of SEQ ID NO: 188 and the VL comprising, consisting essentially of, or consisting of the amino acid sequence of SEQ ID NO: 189. In one embodiment, the CD70×CD3 bispecific antibody comprises, consists essentially of, or consists of the amino acid sequence of SEQ ID NO: 190.

In some embodiments, the bispecific antibody is a CD19×CD3 bispecific antibody, which comprises a first domain that binds to CD19 and a second domain that binds to CD3. In one embodiment, the first domain binds to CD19 and has the CDRs as depicted in SEQ ID NOs: 1 to 6, the second domain binds to CD3 and has the CDRs as depicted in SEQ ID NOs 9 to 14. In another embodiment, the first domain binds to CD19 and has the CDRs as depicted in SEQ ID NOs: 105 to 107 and 109 to 111, the second domain binds to CD3 and has the CDRs as depicted in SEQ ID NOs 9 to 14, and further comprises a HLE domain (third domain). In one embodiment, the CD19×CD3 bispecific antibody comprises, consists essentially of, or consists of the amino acid sequence of SEQ ID NO: 17. In another embodiment, CD19×CD3 bispecific antibody comprises, consists essentially of, or consists of the amino acid sequence of SEQ ID NO: 114.

In some embodiments, the bispecific antibody is a BCMA×CD3 bispecific antibody, which comprises a first domain that binds to BCMA and a second domain that binds to CD3. In some embodiments, the bispecific antibody further comprises a HLE domain (third domain). In one embodiment, the first domain binds to BCMA and has the CDRs as depicted in SEQ ID NOs: 126 to 131, the second domain binds to CD3 and has the CDRs as depicted in SEQ ID NOs 9 to 14. In one embodiment, the BCMA×CD3 bispecific antibody comprises a VH and a VL, wherein the VH comprising, consisting essentially of, or consisting of the amino acid sequence of SEQ ID NO: 132 and the VL comprising, consisting essentially of, or consisting of the amino acid sequence of SEQ ID NO: 133. In one embodiment, the BCMA×CD3 bispecific antibody comprises, consists essentially of, or consists of the amino acid sequence of SEQ ID NO: 135. In another embodiment, the BCMA×CD3 bispecific antibody comprises, consists essentially of, or consists of the amino acid sequence of SEQ ID NO: 136.

In some embodiments, the bispecific antibody is a CD33×CD3 bispecific antibody, which comprises a first domain that binds to CD33 and a second domain that binds to CD3. In some embodiments, the bispecific antibody further comprises a HLE domain (third domain). In one embodiment, the first domain binds to CD33 and has the CDRs as depicted in SEQ ID NOs: 29 to 31 and 34 to 36, the second domain binds to CD3 and has the CDRs as depicted in SEQ ID NOs 9 to 14. In one embodiment, the CD33×CD3 bispecific antibody comprises a VH and a VL, wherein the VH comprising, consisting essentially of, or consisting of the amino acid sequence of SEQ ID NO: 27 or 28 and the VL comprising, consisting essentially of, or consisting of the amino acid sequence of SEQ ID NO: 32 or 33. In one embodiment, the CD33×CD3 bispecific antibody comprises, consists essentially of, or consists of the amino acid sequence of SEQ ID NO: 40. In another embodiment, the BCMA×CD3 bispecific antibody comprises, consists essentially of, or consists of the amino acid sequence of SEQ ID NO: 41.

In some embodiments, the bispecific antibody is a EGFRvIII×CD3 bispecific antibody, which comprises a first domain that binds to EGFRvIII and a second domain that binds to CD3. In some embodiments, the bispecific antibody further comprises a HLE domain (third domain). In one embodiment, the first domain binds to EGFRvIII and has the CDRs as depicted in SEQ ID NOs: 42 to 47, the second domain binds to CD3 and has the CDRs as depicted in SEQ ID NOs 9 to 14. In one embodiment, the EGFRvIII×CD3 bispecific antibody comprises a VH and a VL, wherein the VH comprising, consisting essentially of, or consisting of the amino acid sequence of SEQ ID NO: 48 and the VL comprising, consisting essentially of, or consisting of the amino acid sequence of SEQ ID NO: 49. In one embodiment, the EGFRvIII×CD3 bispecific antibody comprises, consists essentially of, or consists of the amino acid sequence of SEQ ID NO: 52.

In some embodiments, the bispecific antibody is a MSLN×CD3 bispecific antibody, which comprises a first domain that binds to MSLN and a second domain that binds to CD3. In some embodiments, the bispecific antibody further comprises a HLE domain (third domain). In one embodiment, the first domain binds to MSLN and has the CDRs as depicted in SEQ ID NOs: 53 to 58, the second domain binds to CD3 and has the CDRs as depicted in SEQ ID NOs 9 to 14. In one embodiment, the MSLN×CD3 bispecific antibody comprises a VH and a VL, wherein the VH comprising, consisting essentially of, or consisting of the amino acid sequence of SEQ ID NO: 59 and the VL comprising, consisting essentially of, or consisting of the amino acid sequence of SEQ ID NO: 60. In one embodiment, the MSLN×CD3 bispecific antibody comprises, consists essentially of, or consists of the amino acid sequence of SEQ ID NO: 63. In one embodiment, the MSLN×CD3 bispecific antibody comprises, consisting essentially of, or consisting of the amino acid sequence of SEQ ID NO: 64.

In some embodiments, the bispecific antibody is a CDH19×CD3 bispecific antibody, which comprises a first domain that binds to CDH19 and a second domain that binds to CD3. In some embodiments, the bispecific antibody further comprises a HLE domain (third domain). In one embodiment, the first domain binds to CDH19 and has the CDRs as depicted in SEQ ID NOs: 65 to 70, the second domain binds to CD3 and has the CDRs as depicted in SEQ ID NOs 9 to 14. In one embodiment, the CDH19×CD3 bispecific antibody comprises a VH and a VL, wherein the VH comprising, consisting essentially of, or consisting of the amino acid sequence of SEQ ID NO: 71 and the VL comprising, consisting essentially of, or consisting of the amino acid sequence of SEQ ID NO: 72. In one embodiment, the CDH19×CD3 bispecific antibody comprises, consists essentially of, or consists of the amino acid sequence of any of SEQ ID NOs: 74-82. In one embodiment, the CDH19×CD3 bispecific antibody comprises, consists essentially of, or consists of the amino acid sequence of SEQ ID NO: 82.

In some embodiments, the bispecific antibody is a DLL3×CD3 bispecific antibody, which comprises a first domain that binds to DLL3 and a second domain that binds to CD3. In some embodiments, the bispecific antibody further comprises a HLE domain (third domain). In one embodiment, the first domain binds to DLL3 and has the CDRs as depicted in SEQ ID NOs: 94 to 99, the second domain binds to CD3 and has the CDRs as depicted in SEQ ID NOs 9 to 14. In one embodiment, the DLL3×CD3 bispecific antibody comprises a VH and a VL, wherein the VH comprising, consisting essentially of, or consisting of the amino acid sequence of SEQ ID NO: 100 and the VL comprising, consisting essentially of, or consisting of the amino acid sequence of SEQ ID NO: 101. In one embodiment, the DLL3×CD3 bispecific antibody comprises, consists essentially of, or consists of the amino acid sequence of SEQ ID NO: 104.

In some embodiments, the bispecific antibody is a FLT3×CD3 bispecific antibody, which comprises a first domain that binds to FLT3 and a second domain that binds to CD3. In some embodiments, the bispecific antibody further comprises a HLE domain (third domain). In one embodiment, the first domain binds to FLT3 and has the CDRs as depicted in SEQ ID NOs: 83 to 88, the second domain binds to CD3 and has the CDRs as depicted in SEQ ID NOs 9 to 14. In one embodiment, the FLT3×CD3 bispecific antibody comprises a VH and a VL, wherein the VH comprising, consisting essentially of, or consisting of the amino acid sequence of SEQ ID NO: 89 and the VL comprising, consisting essentially of, or consisting of the amino acid sequence of SEQ ID NO: 90. In one embodiment, the FTL3×CD3 bispecific antibody comprises, consists essentially of, or consists of the amino acid sequence of SEQ ID NO: 93.

In some embodiments, the bispecific antibody is a CDH3×CD3 bispecific antibody, which comprises a first domain that binds to CDH3 and a second domain that binds to CD3. In some embodiments, the bispecific antibody further comprises a HLE domain (third domain). In one embodiment, the first domain binds to CDH3 and has the CDRs as depicted in SEQ ID NOs: 115 to 120, the second domain binds to CD3 and has the CDRs as depicted in SEQ ID Nos: 9 to 14. In one embodiment, the CDH3×CD3 bispecific antibody comprises a VH and a VL, wherein the VH comprising, consisting essentially of, or consisting of the amino acid sequence of SEQ ID NO: 121 and the VL comprising, consisting essentially of, or consisting of the amino acid sequence of SEQ ID NO: 122. In one embodiment, the CDH3×CD3 bispecific antibody comprises, consists essentially of, or consists of the amino acid sequence of SEQ ID NO: 125.

In some embodiments, the bispecific antibody is a PSMA×CD3 bispecific antibody, which comprises a first domain that binds to PSMA and a second domain that binds to CD3. In some embodiments, the bispecific antibody further comprises a HLE domain (third domain). In one embodiment, the first domain binds to PSMA and has the CDRs as depicted in SEQ ID NOs: 137 to 142, the second domain binds to CD3 and has the CDRs as depicted in SEQ ID NOs 9 to 14. In one embodiment, the PSMA×CD3 bispecific antibody comprises a VH and a VL, wherein the VH comprising, consisting essentially of, or consisting of the amino acid sequence of SEQ ID NO: 143 and the VL comprising, consisting essentially of, or consisting of the amino acid sequence of SEQ ID NO: 144. In one embodiment, the PSMA×CD3 bispecific antibody comprises, consists essentially of, or consists of the amino acid sequence of any one of SEQ ID NOs: 146 to 151, 161 to 168 and 176 to 181. In one embodiment, the PSMA×CD3 bispecific antibody comprises, consists essentially of, or consists of the amino acid sequence of SEQ ID NO: 177.

The bispecific antibody disclosed herein can be prepared by methods known in the art. For example, the bispecific antibody can be prepared by methods disclosed in WO2008/119657 and WO2017/134140.

In some embodiments, the bispecific antibody is a bispecific masked antigen binding protein. Bispecific masked antigen binding proteins have been previously described. Sec, e.g., International Publication No. WO 2017/040344; U.S. Patent Publication No. 2015/0079088. A “bispecific masked antigen binding protein” or a “masked bispecific binding protein” is understood to refer to a masked antigen binding protein that binds two different antigens or epitopes. A “masked antigen binding protein” is a protein that includes a masking domain (MD) coupled (e.g., through covalent bond or a linker) to an antigen binding domain (AB) such that coupling of the MD inhibits or reduces the binding of the AB to its antigen. The MD further comprises a protein recognition site (PR) that includes a substrate or binding site for a protein or protease, such that when the protein or protease binds to and/or cleaves the protein recognition site the AB domain binds antigen or the binding to the antigen by the AB domain is increased or induced. Masked antigen binding proteins have been previously described in, e.g., International Publication No. WO 2017/040344, U.S. Pat. No. 9,540,440, U.S. Publication No. 20150118254, U.S. Pat. Nos. 9,127,053, 9,517,276, and 8,563,269. It will be apparent to the skilled artisan that in some embodiments a masked antigen binding protein can lack a MD due to cleavage of the PR by a protease, resulting in release of at least the MD (e.g., where the MD is not joined to the masked antigen binding protein by a covalent bond (e.g., a disulfide bond between cysteine residues)).

In some embodiments, the masked antigen binding protein is a Probody (as described in, for example, Polu K R and Lowman H B. Expert Opin Biol Ther. 2014 August; 14 (8): 1049-53; or Desnoyers L R et. al., Sci Transl Med. 2013 Oct. 16;5 (207)) or a ProTIA prodrug (as described in, for example, Schellenberger V. Amunix unveils next-generation immuno-oncologic cancer therapy platform. biopharmadealmakers.nature.com. September 2016 edition, B20; see also www.amunix.com/technology/pro-tia/). In some embodiments, the masked antigen binding protein has the structural arrangement from N-terminus to C-terminus as follows: MD-AB or AB-MD. In some embodiments, the masked antigen binding protein includes a linking peptide (LP), and the masked antigen binding protein has the structural arrangement from N-terminus to C-terminus as follows: MD-LP-AB or AB-LP-MD.

When the masked antigen binding protein is in the presence of the antigen in a naive state, binding of the AB to the antigen is reduced or inhibited, as compared to the binding of the AB to the antigen when the masked antigen binding protein is in the active state (i.e., when a protein or protease binds to the PR and/or removes or dislocates the MD). When compared to the binding of the AB not associated with an MD (i.e., when the masked antigen binding protein is in the active state), the ability of the masked antigen binding protein to bind antigen in the naïve state is reduced, for example, by at least about 30%, about 35%, about 40%, about 45%, about 50%, about 60%, about 70%, about 80%, about 90%, about 92%, about 93%, about 94%, about 95%, about 96%, about 97%, about 98%, about 99%, or even about 100% when measured in vitro and/or in vivo binding assays.

In some embodiments, the protein recognition site (PR) functions as a substrate for a protease, preferably an extracellular protease. The PR may be selected based on a protein or protease that is produced by a cell (including, e.g., tumor cells) in proximity to cells that express the antigen and/or produced by a cell (including, e.g., tumor cells) that is co-localized in tissue with the desired antigen of the AB of the masked antigen binding protein. In some embodiments, the protease is u-type plasminogen activator (uPA, also referred to as urokinase), legumain, and/or matriptase (also referred to as MT-SP1 or MTSP1). In some embodiments, the protease is a matrix metalloprotease (MMP). In some embodiments, the protease is one of the proteases described in Rawlings, N. and Salvesen, G. Handbook of Proteolytic Enzymes (Third Edition). Elsevier, 2013. ISBN: 978-0-12-382219-2.

In some embodiments, one antibody or antigen binding fragment thereof (AB1) domain of a bispecific antigen binding protein has specificity for a target antigen and another antibody or antigen binding fragment thereof (AB2) domain has specificity for another target antigen. In some embodiments, one antibody or antigen binding fragment thereof (AB1) domain of a bispecific antigen binding protein has specificity for an epitope of a target antigen and another antibody or antigen binding fragment thereof (AB2) domain has specificity for another epitope of the same target antigen.

The term “antibody fragment”, “antibody fragment thereof”, or “antigen binding antibody fragment” is understood to refer to a portion of an intact antibody. An “antigen binding fragment” or “antigen binding fragment thereof” refers to a portion of an intact antibody that binds to an antigen. An antigen binding fragment can contain the antigenic determining variable regions of an intact antibody. Examples of antibody fragments antigen binding fragment include, but are not limited to Fab, Fab′, F(ab′)2, and Fv fragments, linear antibodies, scFvs, and single chain antibodies.

A bispecific masked antigen binding protein includes at least one masking domain (MD) comprising a protein recognition site (PR), wherein the MDs inhibit or reduce the binding of AB to an antigen. The at least one MD comprises a protein recognition site (PR) that includes a substrate or binding site for a protein or protease, such that when the protein or protease binds to and/or cleaves the protease recognition site the AB domain binds antigen or binding is increased. For a bispecific masked antigen binding protein, the masked antigen binding protein preferably comprises two MDs (e.g., MD1 and MD2) that reduce the ability of each antigen binding domain (AB1 and AB2) to bind its respective antigen or epitope.

The masked bispecific masked antigen binding proteins provided herein are stable in circulation and activated at intended sites of therapy and/or diagnosis, but not in normal (i.e., healthy) tissue. When in an active state, the masked antigen binding proteins and/or bispecific or multispecific masked antigen binding proteins exhibit binding to an antigen that is at least comparable to the corresponding, unmodified antibody or bispecific or multispecific antigen binding protein (i.e., counterpart antibody which does not comprise a masking domain).

The bispecific masked antigen binding proteins described herein, in various embodiments, bind human CD3. For example, in various aspects, the bispecific masked antigen binding proteins activate T cells via engagement of CD38 on the T cells. That is, the antibodies agonize, stimulate, activate, and/or augment CD3-mediated T cell activation. Biological activities of CD3 include, for example, T cell activation and other signaling through interaction between CD3 and the antigen binding subunits of the T-Cell Receptor (TCR).

The bispecific masked antigen binding proteins disclosed herein optionally bind to CD38 with a binding constant (Kd) of ≤1 μM, for example, in some embodiments, ≤100 nM, ≤10 nM, or ≤1 nM.

In various aspects, the bispecific masked antigen binding protein binds epidermal growth factor receptor (EGFR). The bispecific masked antigen binding proteins disclosed herein optionally bind to human EGFR with a binding constant (Kd) of ≤1 μM, for example, in some embodiments, ≤100 nM, ≤10 nM, or ≤1 nM.

In preferred embodiments, the masked bispecific or multispecific antigen binding protein binds both CD3 and EGFR.

In some embodiments, the masked antigen binding protein is heterodimeric such that it includes an antigen binding domain that is a Fab (e.g., an IgG Fab) and an antigen binding domain that is a scFv. For example, in an exemplary embodiment, the masked antigen binding protein comprises a heavy and light chain (e.g., IgG heavy and light chain) that binds one target (e.g., EGFR) and a scFv domain that binds a second target (e.g., a T-cell surface antigen such as CD3). A single-chain antibody (scFv) is an antigen binding protein in which a VL and a VH region are joined via a linker (e.g., a synthetic sequence of amino acid residues usually about 15 to about 20 amino acids in length) to form a continuous protein chain wherein the linker is long enough to allow the protein chain to fold back on itself and form a monovalent antigen binding site (see, e.g., Bird et al., 1988, Science 242:423-26 and Huston et al., 1988, Proc. Natl. Acad. Sci. USA 85:5879-83). An example of a linker suitable for use in an scFv is GGGGSGGGGSGGGGS (SEQ ID NO: 199). Other exemplary linkers contain at least 4-5 amino acids, ranging from about 4 residues to about 20 residues.

An exemplary format for the masked antigen binding protein comprises (i) two heavy chains comprising an scFv operably attached near the N-terminus of one or both heavy chain(s) and (ii) two light chains which associate with the heavy chains to form an antigen binding domain, wherein the scFv binds CD3 and the Fab portions bind EGFR. In this example, the antigen binding protein comprises three (or four) antigen binding domains, which typically bind two or more different antigens (or epitopes). An example of a linker suitable for linking an scFv to a heavy chain variable region is GGGGS (SEQ ID NO: 197). Exemplary linkers contain at least 1 suitable linker having 4-5 amino acids.

The masked antigen binding protein comprises a masking domain (MD) that is coupled to the antibody (AB) (e.g., via covalent bond or another form of attachment). A masking domain (MD) comprises a masking peptide (or masking polypeptide) (MP) and a protein recognition site (PR). A masking peptide (or masking polypeptide) may be a stretch of amino acids that impedes binding of an antigen binding domain to its antigen. Generally, masking peptides, short sequences of 5-15 amino acids in length, are employed, although shorter and longer sequences (i.e., masking polypeptides) are also contemplated. Masking domains are further described in, e.g., International Publication No. Wo 2017/040344; U.S. Patent Publication No. 2015/0079088 (incorporated by reference in its entirety and in particular with respect to the disclosure of masking domains for use with antibodies that bind EGFR) and U.S. Patent Publication No. 2016/0194399 (incorporated by reference in its entirety and in particular with respect to the disclosure of masking domains for use with antibodies that bind CD3).

In some embodiments, the masking peptide (or masking polypeptide) (MP) is attached to the antigen binding domain (AB) via a protein recognition site (PR), which is optionally part of a larger linker sequence (i.e., a stretch of amino acids connecting the MP to the antigen binding protein). A PR functions as a substrate (or binding site) for a protein or a protease, preferably an extracellular protease. The PR may be selected based on a protein or a protease that is produced by a cell (including, e.g. tumor cells) that is in proximity to cells that express the target and/or produced by a cell (including, e.g. tumor cells) that is co-localized in tissue with the desired target of at least one AB of the masked antigen binding protein. In some embodiments, the protease is u-type plasminogen activator (uPA, also referred to as urokinase), legumain, and/or matriptase (also referred to as MT-SP1 or MTSP1). In some embodiments, the protease is a matrix metalloprotease (MMP). In some embodiments, the protease is one of the proteases described in Rawlings, N. and Salvesen, G. Handbook of Protcolytic Enzymes (Third Edition). Elsevier, 2013. ISBN: 978-0-12-382219-2. Alternatively, the MP is coupled to the antigen binding protein via a non-cleavable protein binding domain. In this regard, the PR is optionally an amino acid sequence that, upon interaction with, e.g., a protein or a protease, is conformationally changed such that the position of the MP is adjusted and the AB is free to bind the target.

In various aspects of the disclosure, a bispecific masked antigen binding protein comprises a MD for each antigen binding portion of the construct (e.g., AB1, AB2, AB3, etc.). For example, a bispecific masked antigen binding protein comprising two Fab portions and two scFvs can comprise two sets of MDs, which can independently be the same or different. To illustrate, a bispecific masked antigen binding protein comprises (i) two scFvs that bind CD3 and a MD1 (optionally the same MD for each scFv) attached to each scFv, and (ii) two Fab portions that bind EGFR and a MD2 attached to each Fab (optionally the same MD for each Fab). In various aspects, the PRs of MD1 and MD2 comprise the same protein recognition sequence, allowing release of the MD for each binding region in the same environment after engagement with a protease. The MD may be attached at the N-terminus or C-terminus of an antigen binding domain, so long as the MD is able to impede binding of the antigen binding domain to the target and the linker does not interfere with binding once the MD is released. When the antigen binding domain is a Fab, the MD may be operably linked to the heavy chain variable region or the light chain variable region. In instances where the bispecific masked antigen binding protein comprises an intact antibody having both a Fab antigen binding domain and an scFv fused to the heavy chain (i.e., a “stacked” conformation), the MD associated with the Fab antigen binding domain is preferably fused to the light chain variable region.

In some embodiments, one AB domain (e.g., AB1) in the bispecific masked antigen binding protein engages a T-cell surface ligand, such as CD3, and optionally takes the form of an scFv. Exemplary masked antigen binding proteins that engage CD3 include, but are not limited to, International Publication No. WO 2017/040344, U.S. Patent Publication No. 20160194399.

In some embodiments, one AB domain (e.g., AB2) in the antigen binding protein engages a tumor antigen, such as EGFR. Exemplary masked antigen binding proteins that engage EGFR include, but are not limited to, those antibodies disclosed in U.S. Patent Publication No. 20150079088.

In some embodiments, the masked antigen binding protein (including, e.g., a bispecific masked antigen binding protein) is modified to alter the isoelectric point of the antibody. For example, in some embodiments, one or more negatively charged, pH sensitive amino acids (e.g., aspartic acid or glutamic acid) in one or both masking domains is substituted with a positively charged amino acid (e.g., a lysine or arginine). In some embodiments, replacing an aspartic acid in one or more masking moieties of the antibody may increase the pI. In some embodiments, the one or more negatively charged, pH sensitive amino acids (e.g., aspartic acid or glutamic acid) in one or both masking domains is removed or substituted with a neutral amino acid.

While the masked antigen binding protein of the disclosure (including, e.g., a bispecific or a multispecific masked antigen binding protein) is often described herein as comprising an intact antibody structure, the disclosure also contemplates the use of antigen binding antibody fragments that lack at least a portion of the traditional two heavy chain/two light chain structure. The fragment employed as a masked antigen binding protein comprises an antigen binding domain (AB) that is operably linked to a masking domain (MD), as described above. For example, in some embodiments, the antigen binding protein comprises two scFvs connected by a suitable linker (e.g., stretch of amino acids of sufficient length to allow each scFv to bind its target). One or both scFvs are attached to a MD; when both scFvs are attached to MDs, the MDs may be the same or different (i.e., the MP and/or PR are different although, in various aspects, it is preferable for the PR to be the same).

Masked bispecific antibodies disclosed herein can be made by methods known in the art, e.g., as described in International Publication No. WO 2017/040344; U.S. Patent Publication No. 2015/0079088; and U.S. Patent Publication No. 2016/0194399.

Table 3 lists the sequences disclosed herein.

TABLE 3

| | | | | |
|---|---|---|---|---|
| 1. | CD19 VL CDR1 | artificial | aa | KASQSVDYDGDSYLN |
| 2. | CD19 VL CDR2 | artificial | aa | DASNLVS |
| 3. | CD19 VL CDR3 | artificial | aa | QQSTEDPWT |
| 4. | CD19 VH CDR1 | artificial | aa | SYWMN |
| 5. | CD19 VH CDR2 | artificial | aa | QIWPGDGDTNYNGKFKG |
| 6. | CD19 VH CDR3 | artificial | aa | RETTTVGRYYYAMDY |
| 7. | CD19 VL | artificial | aa | DIQLTQSPASLAVSLGQRATISCKASQSVDYDGDSYLNWYQ |
| | | | | QIPGQPPKLLIYDASNLVSGIPPRFSGSGSGTDFTLNIHPVEK |
| | | | | VDAATYHCQQSTEDPWTFGGGTKLEIK |
| 8. | CD19 VH | artificial | aa | QVQLQQSGAELVRPGSSVKISCKASGYAFSSYWMNWVKQ |
| | | | | RPGQGLEWIGQIWPGDGDTNYNGKFKGKATLTADESSSTA |
| | | | | YMQLSSLASEDSAVYFCARRETTTVGRYYYAMDYWGQGTT |
| | | | | VTVSS |
| 9. | CD3 VH CDR1 | artificial | aa | RYTMH |
| 10. | CD3 VH CDR2 | artificial | aa | YINPSRGYTNYNQKFKD |
| 11. | CD3 VH CDR3 | artificial | aa | YYDDHYCLDY |
| 12. | CD3 VL CDR1 | artificial | aa | RASSSVSYMN |
| 13. | CD3 VL CDR2 | artificial | aa | DTSKVAS |
| 14. | CD3 VL CDR3 | artificial | aa | QQWSSNPLT |
| 15. | CD3 VH | artificial | aa | DIKLQQSGAELARPGASVKMSCKTSGYTFTRYTMHWVKQR |
| | | | | PGQGLEWIGYINPSRGYTNYNQKFKDKATLTTDKSSSTAYM |
| | | | | QLSSLTSEDSAVYYCARYYDDHYCLDYWGQGTTLTVSS |
| 16. | CD3 VL | artificial | aa | VDDIQLTQSPAIMSASPGEKVTMTCRASSSVSYMNWYQQ |
| | | | | KSGTSPKRWIYDTSKVASGVPYRFSGSGSGTSYSLTISSMEA |
| | | | | EDAATYYCQQWSSNPLTFGAGTKLELK |
| 17. | CD19xCD3 scFv | artificial | aa | DIQLTQSPASLAVSLGQRATISCKASQSVDYDGDSYLNWYQ |
| | BLINCYTO incl | | | QIPGQPPKLLIYDASNLVSGIPPRFSGSGSGTDFTLNIHPVEK |
| | linker and his-tag | | | VDAATYHCQQSTEDPWTFGGGTKLEIKGGGGSGGGGSGG |
| | | | | GGSQVQLQQSGAELVRPGSSVKISCKASGYAFSSYWMNW |
| | | | | VKQRPGQGLEWIGQIWPGDGDTNYNGKFKGKATLTADES |
| | | | | SSTAYMQLSSLASEDSAVYFCARRETTTVGRYYYAMDYWG |
| | | | | QGTTVTVSSGGGGSDIKLQQSGAELARPGASVKMSCKTSG |
| | | | | YTFTRYTMHWVKQRPGQGLEWIGYINPSRGYTNYNQKFK |
| | | | | DKATLTTDKSSSTAYMQLSSLTSEDSAVYYCARYYDDHYCLD |
| | | | | YWGQGTTLTVSSVEGGSGGSGGSGGSGGVDDIQLTQSPAI |
| | | | | MSASPGEKVTMTCRASSSVSYMNWYQQKSGTSPKRWIYD |
| | | | | TSKVASGVPYRFSGSGSGTSYSLTISSMEAEDAATYYCQQW |
| | | | | SSNPLTFGAGTKLELKHHHHHH |
| 18. | CDR-L1 I2C | artificial | aa | GSSTGAVTSGNYPN |
| 19. | CDR-L2 12C | artificial | aa | GTKFLAP |
| 20. | CDR-L3 12C | artificial | aa | VLWYSNRWV |
| 21. | CDR-H1 I2C | artificial | aa | KYAMN |
| 22. | CDR-H2 12C | artificial | aa | RIRSKYNNYATYYADSVKD |
| 23. | CDR-H3 12C | artificial | aa | HGNFGNSYISYWAY |
| 24. | VH 12C | artificial | aa | EVQLVESGGGLVQPGGSLKLSCAASGFTFNKYAMNWVRQ |
| | | | | APGKGLEWVARIRSKYNNYATYYADSVKDRFTISRDDSKNT |
| | | | | AYLQMNNLKTEDTAVYYCVRHGNFGNSYISYWAYWGQGT |
| | | | | LVTVSS |
| 25. | VL 12C | artificial | aa | QTVVTQEPSLTVSPGGTVTLTCGSSTGAVTSGNYPNWVQQ |
| | | | | KPGQAPRGLIGGTKFLAPGTPARFSGSLLGGKAALTLSGVQP |
| | | | | EDEAEYYCVLWYSNRWVFGGGTKLTVL |
| 26. | VH-VL 12C | artificial | aa | EVQLVESGGGLVQPGGSLKLSCAASGFTFNKYAMNWVRQ |
| | | | | APGKGLEWVARIRSKYNNYATYYADSVKDRFTISRDDSKNT |
| | | | | AYLQMNNLKTEDTAVYYCVRHGNFGNSYISYWAYWGQGT |
| | | | | LVTVSSGGGGSGGGGSGGGGSQTVVTQEPSLTVSPGGTVT |
| | | | | LTCGSSTGAVTSGNYPNWVQQKPGQAPRGLIGGTKFLAPG |
| | | | | TPARFSGSLLGGKAALTLSGVQPEDEAEYYCVLWYSNRWVF |
| | | | | GGGTKLTVL |
| 27. | CD33 ccVH E11 | artificial | aa | QVQLVQSGAEVKKPGESVKVSCKASGYTFTNYGMNWVKQ |
| | | | | APGQCLEWMGWINTYTGEPTYADKFQGRVTMTTDTSTST |
| | | | | AYMEIRNLGGDDTAVYYCARWSWSDGYYVYFDYWGQGTS |
| | | | | VTVSS |
| 28. | CD33 VH E11 | Artificial | aa | QVQLVQSGAEVKKPGESVKVSCKASGYTFTNYGMNWVKQ |
| | | | | APGQGLEWMGWINTYTGEPTYADKFQGRVTMTTDTSTST |
| | | | | AYMEIRNLGGDDTAVYYCARWSWSDGYYVYFDYWGQGTS |
| | | | | VTVSS |
| 29. | CD33 HCDR1 E11 | artificial | aa | NYGMN |
| 30. | CD33 HCDR2 E11 | artificial | aa | WINTYTGEPTYADKFQG |
| 31. | CD33 HCDR3 E11 | artificial | aa | WSWSDGYYVYFDY |
| 32. | CD33 CC VL E11 | artificial | aa | DIVMTQSPDSLTVSLGERTTINCKSSQSVLDSSTNKNSLAWY |
| | | | | QQKPGQPPKLLLSWASTRESGIPDRFSGSGSGTDFTLTIDSP |
| | | | | QPEDSATYYCQQSAHFPITFGCGTRLEIK |
| 33. | CD33 VL E11 | artificial | aa | DIVMTQSPDSLTVSLGERTTINCKSSQSVLDSSTNKNSLAWY |
| | | | | QQKPGQPPKLLLSWASTRESGIPDRFSGSGSGTDFTLTIDSP |
| | | | | QPEDSATYYCQQSAHFPITFGQGTRLEIK |
| 34. | CD33 LCDR1 E11 | artificial | aa | KSSQSVLDSSTNKNSLA |
| 35. | CD33 LCDR2 E11 | artificial | aa | WASTRES |
| 36. | CD33 LCDR3 E11 | artificial | aa | QQSAHFPIT |
| 37. | CD33 HL CC E11 | artificial | aa | QVQLVQSGAEVKKPGESVKVSCKASGYTFTNYGMNWVKQ |
| | | | | APGQCLEWMGWINTYTGEPTYADKFQGRVTMTTDTSTST |
| | | | | AYMEIRNLGGDDTAVYYCARWSWSDGYYVYFDYWGQGTS |
| | | | | VTVSSGGGGSGGGGSGGGGSDIVMTQSPDSLTVSLGERTTI |
| | | | | NCKSSQSVLDSSTNKNSLAWYQQKPGQPPKLLLSWASTRES |
| | | | | GIPDRFSGSGSGTDFTLTIDSPQPEDSATYYCQQSAHFPITFG |
| | | | | CGTRLEIK |
| 38. | CD33 HL E11 | artificial | aa | QVQLVQSGAEVKKPGESVKVSCKASGYTFTNYGMNWVKQ |
| | | | | APGQGLEWMGWINTYTGEPTYADKFQGRVTMTTDTSTST |
| | | | | AYMEIRNLGGDDTAVYYCARWSWSDGYYVYFDYWGQGTS |
| | | | | VTVSSGGGGSGGGGSGGGGSDIVMTQSPDSLTVSLGERTTI |
| | | | | NCKSSQSVLDSSTNKNSLAWYQQKPGQPPKLLLSWASTRES |
| | | | | GIPDRFSGSGSGTDFTLTIDSPQPEDSATYYCQQSAHFPITFG |
| | | | | QGTRLEIK |
| 39. | CD33 CC E11 HL x | artificial | aa | QVQLVQSGAEVKKPGESVKVSCKASGYTFTNYGMNWVKQ |
| | 12C HL Bispecific | | | APGQCLEWMGWINTYTGEPTYADKFQGRVTMTTDTSTST |
| | molecule | | | AYMEIRNLGGDDTAVYYCARWSWSDGYYVYFDYWGQGTS |
| | | | | VTVSSGGGGSGGGGSGGGGSDIVMTQSPDSLTVSLGERTTI |
| | | | | NCKSSQSVLDSSTNKNSLAWYQQKPGQPPKLLLSWASTRES |
| | | | | GIPDRFSGSGSGTDFTLTIDSPQPEDSATYYCQQSAHFPITFG |
| | | | | CGTRLEIKSGGGGSEVQLVESGGGLVQPGGSLKLSCAASGFT |
| | | | | FNKYAMNWVRQAPGKGLEWVARIRSKYNNYATYYADSVK |
| | | | | DRFTISRDDSKNTAYLQMNNLKTEDTAVYYCVRHGNFGNS |
| | | | | YISYWAYWGQGTLVTVSSGGGGSGGGGSGGGGSQTVVTQ |
| | | | | EPSLTVSPGGTVTLTCGSSTGAVTSGNYPNWVQQKPGQAP |
| | | | | RGLIGGTKFLAPGTPARFSGSLLGGKAALTLSGVQPEDEAEY |
| | | | | YCVLWYSNRWVFGGGTKLTVL |
| 40. | CD33 E11 HL x | artificial | aa | MGWSCIILFLVATATGVHSQVQLVQSGAEVKKPGESVKVSC |
| | 12C HL | | | KASGYTFTNYGMNWVKQAPGQGLEWMGWINTYTGEPTY |
| | | | | ADKFQGRVTMTTDTSTSTAYMEIRNLGGDDTAVYYCARWS |
| | | | | WSDGYYVYFDYWGQGTSVTVSSGGGGSGGGGSGGGGSDI |
| | | | | VMTQSPDSLTVSLGERTTINCKSSQSVLDSSTNKNSLAWYQ |
| | | | | QKPGQPPKLLLSWASTRESGIPDRFSGSGSGTDFTLTIDSPQ |
| | | | | PEDSATYYCQQSAHFPITFGQGTRLEIKSGGGGSEVQLVESG |
| | | | | GGLVQPGGSLKLSCAASGFTFNKYAMNWVRQAPGKGLEW |
| | | | | VARIRSKYNNYATYYADSVKDRFTISRDDSKNTAYLQMNNL |
| | | | | KTEDTAVYYCVRHGNFGNSYISYWAYWGQGTLVTVSSGGG |
| | | | | GSGGGGSGGGGSQTVVTQEPSLTVSPGGTVTLTCGSSTGA |
| | | | | VTSGNYPNWVQQKPGQAPRGLIGGTKFLAPGTPARFSGSL |
| | | | | LGGKAALTLSGVQPEDEAEYYCVLWYSNRWVFGGGTKLTV |
| | | | | LHHHHHH |
| 41. | CD33 CC x 12C- | artificial | aa | QVQLVQSGAEVKKPGESVKVSCKASGYTFTNYGMNWVKQ |
| | scFc Bispecific | | | APGQCLEWMGWINTYTGEPTYADKFQGRVTMTTDTSTST |
| | HLE molecule | | | AYMEIRNLGGDDTAVYYCARWSWSDGYYVYFDYWGQGT |
| | | | | SVTVSSGGGGSGGGGSGGGGSDIVMTQSPDSLTVSLGERT |
| | | | | TINCKSSQSVLDSSTNKNSLAWYQQKPGQPPKLLLSWASTR |
| | | | | ESGIPDRFSGSGSGTDFTLTIDSPQPEDSATYYCQQSAHFPIT |
| | | | | FGCGTRLEIKSGGGGSEVQLVESGGGLVQPGGSLKLSCAAS |
| | | | | GFTFNKYAMNWVRQAPGKGLEWVARIRSKYNNYATYYAD |
| | | | | SVKDRFTISRDDSKNTAYLQMNNLKTEDTAVYYCVRHGNF |
| | | | | GNSYISYWAYWGQGTLVTVSSGGGGSGGGGSGGGGSQT |
| | | | | VVTQEPSLTVSPGGTVTLTCGSSTGAVTSGNYPNWVQQKP |
| | | | | GQAPRGLIGGTKFLAPGTPARFSGSLLGGKAALTLSGVQPE |
| | | | | DEAEYYCVLWYSNRWVFGGGTKLTVLGGGGDKTHTCPPC |
| | | | | PAPELLGGPSVFLFPPKPKDTLMISRTPEVTCVVVDVSHEDP |
| | | | | EVKFNWYVDGVEVHNAKTKPCEEQYGSTYRCVSVLTVLHQ |
| | | | | DWLNGKEYKCKVSNKALPAPIEKTISKAKGQPREPQVYTLPP |
| | | | | SREEMTKNQVSLTCLVKGFYPSDIAVEWESNGQPENNYKT |
| | | | | TPPVLDSDGSFFLYSKLTVDKSRWQQGNVFSCSVMHEALH |
| | | | | NHYTQKSLSLSPGKGGGGSGGGGSGGGGSGGGGSGGGG |
| | | | | SGGGGSDKTHTCPPCPAPELLGGPSVFLFPPKPKDTLMISRT |
| | | | | PEVTCVVVDVSHEDPEVKFNWYVDGVEVHNAKTKPCEEQ |
| | | | | YGSTYRCVSVLTVLHQDWLNGKEYKCKVSNKALPAPIEKTIS |
| | | | | KAKGQPREPQVYTLPPSREEMTKNQVSLTCLVKGFYPSDIA |
| | | | | VEWESNGQPENNYKTTPPVLDSDGSFFLYSKLTVDKSRWQ |
| | | | | QGNVFSCSVMHEALHNHYTQKSLSLSPGK |
| 42. | EGFRvIIIxCD3- | artificial | aa | NYGMH |
| | scFc VH CDR1 | | | |
| 43. | EGFRvIIIxCD3- | artificial | aa | VIWYDGSDKYYADSVRG |
| | scFc VH CDR2 | | | |
| 44. | EGFRvIIIxCD3- | artificial | aa | DGYDILTGNPRDFDY |
| | scFc VH CDR3 | | | |
| 45. | EGFRvIIIxCD3- | artificial | aa | RSSQSLVHSDGNTYLS |
| | scFc VL CDR1 | | | |
| 46. | EGFRvIIIxCD3- | artificial | aa | RISRRFS |
| | scFc VL CDR2 | | | |
| 47. | EGFRvIIIxCD3- | artificial | aa | MQSTHVPRT |
| | scFc VL CDR3 | | | |
| 48. | EGFRvIII_CCxCD3- | artificial | aa | QVQLVESGGGVVQSGRSLRLSCAASGFTFRNYGMHWVRQ |
| | scFc VH | | | APGKCLEWVAVIWYDGSDKYYADSVRGRFTISRDNSKNTLY |
| | | | | LQMNSLRAEDTAVYYCARDGYDILTGNPRDFDYWGQGTL |
| | | | | VTVSS |
| 49. | EGFRvIII_CCxCD3- | artificial | aa | DTVMTQTPLSSHVTLGQPASISCRSSQSLVHSDGNTYLSWL |
| | scFc VL | | | QQRPGQPPRLLIYRISRRFSGVPDRFSGSGAGTDFTLEISRVE |
| | | | | AEDVGVYYCMQSTHVPRTFGCGTKVEIK |
| 50. | EGFRvIII_CCxCD3- | artificial | aa | QVQLVESGGGVVQSGRSLRLSCAASGFTFRNYGMHWVRQ |
| | scFc scFv | | | APGKCLEWVAVIWYDGSDKYYADSVRGRFTISRDNSKNTLY |
| | | | | LQMNSLRAEDTAVYYCARDGYDILTGNPRDFDYWGQGTL |
| | | | | VTVSSGGGGSGGGGSGGGGSDTVMTQTPLSSHVTLGQPA |
| | | | | SISCRSSQSLVHSDGNTYLSWLQQRPGQPPRLLIYRISRRFSG |
| | | | | VPDRFSGSGAGTDFTLEISRVEAEDVGVYYCMQSTHVPRTF |
| | | | | GCGTKVEIK |
| 51. | EGFRvIII_CCxCD3- | artificial | aa | QVQLVESGGGVVQSGRSLRLSCAASGFTFRNYGMHWVRQ |
| | scFc Bispecific | | | APGKCLEWVAVIWYDGSDKYYADSVRGRFTISRDNSKNTLY |
| | molecule | | | LQMNSLRAEDTAVYYCARDGYDILTGNPRDFDYWGQGTL |
| | | | | VTVSSGGGGSGGGGSGGGGSDTVMTQTPLSSHVTLGQPA |
| | | | | SISCRSSQSLVHSDGNTYLSWLQQRPGQPPRLLIYRISRRFSG |
| | | | | VPDRFSGSGAGTDFTLEISRVEAEDVGVYYCMQSTHVPRTF |
| | | | | GCGTKVEIKSGGGGSEVQLVESGGGLVQPGGSLKLSCAASG |
| | | | | FTFNKYAMNWVRQAPGKGLEWVARIRSKYNNYATYYADS |
| | | | | VKDRFTISRDDSKNTAYLQMNNLKTEDTAVYYCVRHGNFG |
| | | | | NSYISYWAYWGQGTLVTVSSGGGGSGGGGSGGGGSQTV |
| | | | | VTQEPSLTVSPGGTVTLTCGSSTGAVTSGNYPNWVQQKPG |
| | | | | QAPRGLIGGTKFLAPGTPARFSGSLLGGKAALTLSGVQPEDE |
| | | | | AEYYCVLWYSNRWVFGGGTKLTVL |
| 52. | EGFRvIII_CCxCD3- | artificial | aa | QVQLVESGGGVVQSGRSLRLSCAASGFTFRNYGMHWVRQ |
| | scFc Bispecific | | | APGKCLEWVAVIWYDGSDKYYADSVRGRFTISRDNSKNTLY |
| | HLE molecule | | | LQMNSLRAEDTAVYYCARDGYDILTGNPRDFDYWGQGTL |
| | | | | VTVSSGGGGSGGGGSGGGGSDTVMTQTPLSSHVTLGQPA |
| | | | | SISCRSSQSLVHSDGNTYLSWLQQRPGQPPRLLIYRISRRFSG |
| | | | | VPDRFSGSGAGTDFTLEISRVEAEDVGVYYCMQSTHVPRTF |
| | | | | GCGTKVEIKSGGGGSEVQLVESGGGLVQPGGSLKLSCAASG |
| | | | | FTFNKYAMNWVRQAPGKGLEWVARIRSKYNNYATYYADS |
| | | | | VKDRFTISRDDSKNTAYLQMNNLKTEDTAVYYCVRHGNFG |
| | | | | NSYISYWAYWGQGTLVTVSSGGGGSGGGGSGGGGSQTV |
| | | | | VTQEPSLTVSPGGTVTLTCGSSTGAVTSGNYPNWVQQKPG |
| | | | | QAPRGLIGGTKFLAPGTPARFSGSLLGGKAALTLSGVQPEDE |
| | | | | AEYYCVLWYSNRWVFGGGTKLTVLGGGGDKTHTCPPCPA |
| | | | | PELLGGPSVFLFPPKPKDTLMISRTPEVTCVVVDVSHEDPEV |
| | | | | KFNWYVDGVEVHNAKTKPCEEQYGSTYRCVSVLTVLHQD |
| | | | | WLNGKEYKCKVSNKALPAPIEKTISKAKGQPREPQVYTLPPS |
| | | | | REEMTKNQVSLTCLVKGFYPSDIAVEWESNGQPENNYKTT |
| | | | | PPVLDSDGSFFLYSKLTVDKSRWQQGNVFSCSVMHEALHN |
| | | | | HYTQKSLSLSPGKGGGGGGGGSGGGGSGGGGSGGGGS |
| | | | | GGGGSDKTHTCPPCPAPELLGGPSVFLFPPKPKDTLMISRTP |
| | | | | EVTCVVVDVSHEDPEVKFNWYVDGVEVHNAKTKPCEEQY |
| | | | | GSTYRCVSVLTVLHQDWLNGKEYKCKVSNKALPAPIEKTISK |
| | | | | AKGQPREPQVYTLPPSREEMTKNQVSLTCLVKGFYPSDIAV |
| | | | | EWESNGQPENNYKTTPPVLDSDGSFFLYSKLTVDKSRWQQ |
| | | | | GNVFSCSVMHEALHNHYTQKSLSLSPGK |
| 53. | MSLN_5 VH | artificial | aa | DYYMT |
| | CDR1 | | | |
| 54. | MSLN_5 VH | artificial | aa | YISSSGSTIYYADSVKG |
| | CDR2 | | | |
| 55. | MSLN_5 VH | artificial | aa | DRNSHFDY |
| | CDR3 | | | |
| 56. | MSLN_5 VL CDR1 | artificial | aa | RASQGINTWLA |
| 57. | MSLN_5 VL CDR2 | artificial | aa | GASGLQS |
| 58. | MSLN_5 VL CDR3 | artificial | aa | QQAKSFPRT |
| 59. | MSLN _5 VH | artificial | aa | QVQLVESGGGLVKPGGSLRLSCAASGFTFSDYYMTWIRQA |
| | | | | PGKGLEWLSYISSSGSTIYYADSVKGRFTISRDNAKNSLFLQM |
| | | | | NSLRAEDTAVYYCARDRNSHFDYWGQGTLVTVSS |
| 60. | MSLN_5 VL | artificial | aa | DIQMTQSPSSVSASVGDRVTITCRASQGINTWLAWYQQKP |
| | | | | GKAPKLLIYGASGLQSGVPSRFSGSGSGTDFTLTISSLQPEDF |
| | | | | ATYYCQQAKSFPRTFGQGTKVEIK |
| 61. | MSLN_5 scFv | artificial | aa | QVQLVESGGGLVKPGGSLRLSCAASGFTFSDYYMTWIRQA |
| | | | | PGKGLEWLSYISSSGSTIYYADSVKGRFTISRDNAKNSLFLQM |
| | | | | NSLRAEDTAVYYCARDRNSHFDYWGQGTLVTVSSGGGGS |
| | | | | GGGGSGGGGSDIQMTQSPSSVSASVGDRVTITCRASQGIN |
| | | | | TWLAWYQQKPGKAPKLLIYGASGLQSGVPSRFSGSGSGTD |
| | | | | FTLTISSLQPEDFATYYCQQAKSFPRTFGQGTKVEIK |
| 62. | MSLN_5x12C0 | artificial | aa | QVQLVESGGGLVKPGGSLRLSCAASGFTFSDYYMTWIRQA |
| | bispecific | | | PGKGLEWLSYISSSGSTIYYADSVKGRFTISRDNAKNSLFLQM |
| | molecule | | | NSLRAEDTAVYYCARDRNSHFDYWGQGTLVTVSSGGGGS |
| | | | | GGGGSGGGGSDIQMTQSPSSVSASVGDRVTITCRASQGIN |
| | | | | TWLAWYQQKPGKAPKLLIYGASGLQSGVPSRFSGSGSGTD |
| | | | | FTLTISSLQPEDFATYYCQQAKSFPRTFGQGTKVEIKSGGGG |
| | | | | SEVQLVESGGGLVQPGGSLKLSCAASGFTFNKYAMNWVRQ |
| | | | | APGKGLEWVARIRSKYNNYATYYADSVKDRFTISRDDSKNT |
| | | | | AYLQMNNLKTEDTAVYYCVRHGNFGNSYISYWAYWGQGT |
| | | | | LVTVSSGGGGSGGGGSGGGGSQTVVTQEPSLTVSPGGTVT |
| | | | | LTCGSSTGAVTSGNYPNWVQQKPGQAPRGLIGGTKFLAPG |
| | | | | TPARFSGSLLGGKAALTLSGVQPEDEAEYYCVLWYSNRWVF |
| | | | | GGGTKLTVL |
| 63. | MSLN_5xCD3- | artificial | aa | QVQLVESGGGLVKPGGSLRLSCAASGFTFSDYYMTWIRQA |
| | scFc Bispecific | | | PGKGLEWLSYISSSGSTIYYADSVKGRFTISRDNAKNSLFLQM |
| | HLE molecule | | | NSLRAEDTAVYYCARDRNSHFDYWGQGTLVTVSSGGGGS |
| | | | | GGGGSGGGGSDIQMTQSPSSVSASVGDRVTITCRASQGIN |
| | | | | TWLAWYQQKPGKAPKLLIYGASGLQSGVPSRFSGSGSGTD |
| | | | | FTLTISSLQPEDFATYYCQQAKSFPRTFGQGTKVEIKSGGGG |
| | | | | SEVQLVESGGGLVQPGGSLKLSCAASGFTFNKYAMNWVRQ |
| | | | | APGKGLEWVARIRSKYNNYATYYADSVKDRFTISRDDSKNT |
| | | | | AYLQMNNLKTEDTAVYYCVRHGNFGNSYISYWAYWGQGT |
| | | | | LVTVSSGGGGSGGGGSGGGGSQTVVTQEPSLTVSPGGTVT |
| | | | | LTCGSSTGAVTSGNYPNWVQQKPGQAPRGLIGGTKFLAPG |
| | | | | TPARFSGSLLGGKAALTLSGVQPEDEAEYYCVLWYSNRWVF |
| | | | | GGGTKLTVLGGGGDKTHTCPPCPAPELLGGPSVFLFPPKPK |
| | | | | DTLMISRTPEVTCVVVDVSHEDPEVKFNWYVDGVEVHNAK |
| | | | | TKPCEEQYGSTYRCVSVLTVLHQDWLNGKEYKCKVSNKALP |
| | | | | APIEKTISKAKGQPREPQVYTLPPSREEMTKNQVSLTCLVKG |
| | | | | FYPSDIAVEWESNGQPENNYKTTPPVLDSDGSFFLYSKLTVD |
| | | | | KSRWQQGNVFSCSVMHEALHNHYTQKSLSLSPGKGGGGS |
| | | | | GGGGSGGGGSGGGGSGGGGSGGGGSDKTHTCPPCPAPEL |
| | | | | LGGPSVFLFPPKPKDTLMISRTPEVTCVVVDVSHEDPEVKFN |
| | | | | WYVDGVEVHNAKTKPCEEQYGSTYRCVSVLTVLHQDWLN |
| | | | | GKEYKCKVSNKALPAPIEKTISKAKGQPREPQVYTLPPSREE |
| | | | | MTKNQVSLTCLVKGFYPSDIAVEWESNGQPENNYKTTPPVL |
| | | | | DSDGSFFLYSKLTVDKSRWQQGNVFSCSVMHEALHNHYTQ |
| | | | | KSLSLSPGK |
| 64. | MSLN_5_CCxCD3- | artificial | aa | QVQLVESGGGLVKPGGSLRLSCAASGFTFSDHYMSWIRQA |
| | scFc Bispecific | | | PGKCLEWFSYISSSGGIIYYADSVKGRFTISRDNAKNSLYLQM |
| | HLE molecule | | | NSLRAEDTAVYYCARDVGSHFDYWGQGTLVTVSSGGGGS |
| | | | | GGGGSGGGGSDIQMTQSPSSVSASVGDRVTITCRASQDISR |
| | | | | WLAWYQQKPGKAPKLLISAASRLQSGVPSRFSGSGSGTDFT |
| | | | | LTISSLQPEDFAIYYCQQAKSFPRTFGCGTKVEIKSGGGGSEV |
| | | | | QLVESGGGLVQPGGSLKLSCAASGFTFNKYAMNWVRQAP |
| | | | | GKGLEWVARIRSKYNNYATYYADSVKDRFTISRDDSKNTAYL |
| | | | | QMNNLKTEDTAVYYCVRHGNFGNSYISYWAYWGQGTLVT |
| | | | | VSSGGGGGGGGSGGGGSQTVVTQEPSLTVSPGGTVTLTC |
| | | | | GSSTGAVTSGNYPNWVQQKPGQAPRGLIGGTKFLAPGTPA |
| | | | | RFSGSLLGGKAALTLSGVQPEDEAEYYCVLWYSNRWVFGG |
| | | | | GTKLTVLGGGGDKTHTCPPCPAPELLGGPSVFLFPPKPKDTL |
| | | | | MISRTPEVTCVVVDVSHEDPEVKFNWYVDGVEVHNAKTKP |
| | | | | CEEQYGSTYRCVSVLTVLHQDWLNGKEYKCKVSNKALPAPI |
| | | | | EKTISKAKGQPREPQVYTLPPSREEMTKNQVSLTCLVKGFYP |
| | | | | SDIAVEWESNGQPENNYKTTPPVLDSDGSFFLYSKLTVDKSR |
| | | | | WQQGNVFSCSVMHEALHNHYTQKSLSLSPGKGGGGSGG |
| | | | | GGSGGGGSGGGGSGGGGSGGGGSDKTHTCPPCPAPELLG |
| | | | | GPSVFLFPPKPKDTLMISRTPEVTCVVVDVSHEDPEVKFNW |
| | | | | YVDGVEVHNAKTKPCEEQYGSTYRCVSVLTVLHQDWLNGK |
| | | | | EYKCKVSNKALPAPIEKTISKAKGQPREPQVYTLPPSREEMTK |
| | | | | NQVSLTCLVKGFYPSDIAVEWESNGQPENNYKTTPPVLDSD |
| | | | | GSFFLYSKLTVDKSRWQQGNVFSCSVMHEALHNHYTQKSL |
| | | | | SLSPGK |
| 65. | CDR-H1 CDH19 | artificial | aa | SYGMH |
| | 65254.007 | | | |
| 66. | CDR-H2 CDH19 | artificial | aa | FIWYEGSNKYYAESVKD |
| | 65254.007 | | | |
| 67. | CDR-H3 CDH19 | artificial | aa | RAGIIGTIGYYYGMDV |
| | 65254.007 | | | |
| 68. | CDR-L1 CDH19 | artificial | aa | SGDRLGEKYTS |
| | 65254.007 | | | |
| 69. | CDR-L2 CDH19 | artificial | aa | QDTKRPS |
| | 65254.007 | | | |
| 70. | CDR-L3 CDH19 | artificial | aa | QAWESSTVV |
| | 65254.007 | | | |
| 71. | VH CDH19 | artificial | aa | QVQLVESGGGVVQPGGSLRLSCAASGFTFSSYGMHWVRQ |
| | 65254.007 | | | APGKGLEWVAFIWYEGSNKYYAESVKDRFTISRDNSKNTLYL |
| | | | | QMNSLRAEDTAVYYCARRAGIIGTIGYYYGMDVWGQGTTV |
| | | | | TVSS |
| 72. | VL CDH19 | artificial | aa | SYELTQPPSVSVSPGQTASITCSGDRLGEKYTSWYQQRPGQ |
| | 65254.007 | | | SPLLVIYQDTKRPSGIPERFSGSNSGNTATLTISGTQAMDEA |
| | | | | DYYCQAWESSTVVFGGGTKLTVLS |
| 73. | VH-VL CDH19 | artificial | aa | QVQLVESGGGVVQPGGSLRLSCAASGFTFSSYGMHWVRQ |
| | 65254.007 | | | APGKGLEWVAFIWYEGSNKYYAESVKDRFTISRDNSKNTLYL |
| | | | | QMNSLRAEDTAVYYCARRAGIIGTIGYYYGMDVWGQGTTV |
| | | | | TVSSGGGGSGGGGSGGGGSSYELTQPPSVSVSPGQTASITC |
| | | | | SGDRLGEKYTSWYQQRPGQSPLLVIYQDTKRPSGIPERFSGS |
| | | | | NSGNTATLTISGTQAMDEADYYCQAWESSTVVFGGGTKLT |
| | | | | VLS |
| 74. | CDH19 | artificial | aa | QVQLVESGGGVVQPGGSLRLSCAASGFTFSSYGMHWVRQ |
| | 65254.007 x 12C | | | APGKGLEWVAFIWYEGSNKYYAESVKDRFTISRDNSKNTLYL |
| | | | | QMNSLRAEDTAVYYCARRAGIIGTIGYYYGMDVWGQGTTV |
| | | | | TVSSGGGGSGGGGSGGGGSSYELTQPPSVSVSPGQTASITC |
| | | | | SGDRLGEKYTSWYQQRPGQSPLLVIYQDTKRPSGIPERFSGS |
| | | | | NSGNTATLTISGTQAMDEADYYCQAWESSTVVFGGGTKLT |
| | | | | VLSGGGGSEVQLVESGGGLVQPGGSLKLSCAASGFTFNKYA |
| | | | | MNWVRQAPGKGLEWVARIRSKYNNYATYYADSVKDRFTIS |
| | | | | RDDSKNTAYLQMNNLKTEDTAVYYCVRHGNFGNSYISYWA |
| | | | | YWGQGTLVTVSSGGGGSGGGGSGGGGSQTVVTQEPSLTV |
| | | | | SPGGTVTLTCGSSTGAVTSGNYPNWVQQKPGQAPRGLIGG |
| | | | | TKFLAPGTPARFSGSLLGGKAALTLSGVQPEDEAEYYCVLWY |
| | | | | SNRWVFGGGTKLTVLHHHHHH |
| 75. | CDH19 | artificial | aa | QVQLVESGGGVVQPGGSLRLSCAASGFTFSSYGMHWVRQ |
| | 65254.007 x 12C- | | | APGKGLEWVAFIWYEGSNKYYAESVKDRFTISRDNSKNTLY |
| | scFc Bispecific | | | LQMNSLRAEDTAVYYCARRAGIIGTIGYYYGMDVWGQGTT |
| | HLE molecule | | | VTVSSGGGGSGGGGSGGGGSSYELTQPPSVSVSPGQTASIT |
| | | | | CSGDRLGEKYTSWYQQRPGQSPLLVIYQDTKRPSGIPERFS |
| | | | | GSNSGNTATLTISGTQAMDEADYYCQAWESSTVVFGGGTK |
| | | | | LTVLSGGGGSEVQLVESGGGLVQPGGSLKLSCAASGFTFNK |
| | | | | YAMNWVRQAPGKGLEWVARIRSKYNNYATYYADSVKDRF |
| | | | | TISRDDSKNTAYLQMNNLKTEDTAVYYCVRHGNFGNSYISY |
| | | | | WAYWGQGTLVTVSSGGGGSGGGGSGGGGSQTVVTQEPS |
| | | | | LTVSPGGTVTLTCGSSTGAVTSGNYPNWVQQKPGQAPRG |
| | | | | LIGGTKFLAPGTPARFSGSLLGGKAALTLSGVQPEDEAEYYC |
| | | | | VLWYSNRWVFGGGTKLTVLGGGGDKTHTCPPCPAPELLG |
| | | | | GPSVFLFPPKPKDTLMISRTPEVTCVVVDVSHEDPEVKFNW |
| | | | | YVDGVEVHNAKTKPCEEQYGSTYRCVSVLTVLHQDWLNGK |
| | | | | EYKCKVSNKALPAPIEKTISKAKGQPREPQVYTLPPSREEMT |
| | | | | KNQVSLTCLVKGFYPSDIAVEWESNGQPENNYKTTPPVLDS |
| | | | | DGSFFLYSKLTVDKSRWQQGNVFSCSVMHEALHNHYTQKS |
| | | | | LSLSPGKGGGGSGGGGSGGGGSGGGGSGGGGGGGGSD |
| | | | | KTHTCPPCPAPELLGGPSVFLFPPKPKDTLMISRTPEVTCVV |
| | | | | VDVSHEDPEVKFNWYVDGVEVHNAKTKPCEEQYGSTYRC |
| | | | | VSVLTVLHQDWLNGKEYKCKVSNKALPAPIEKTISKAKGQP |
| | | | | REPQVYTLPPSREEMTKNQVSLTCLVKGFYPSDIAVEWESN |
| | | | | GQPENNYKTTPPVLDSDGSFFLYSKLTVDKSRWQQGNVFS |
| | | | | CSVMHEALHNHYTQKSLSLSPGK |
| 76. | CDH19 | artificial | aa | QVQLVESGGGVVQPGGSLRLSCAASGFTFSSYGMHWVRQ |
| | 65254.007 x 12C- | | | APGKGLEWVAFIWYEGSNKYYAESVKDRFTISRDNSKNTLY |
| | scFc_delGK | | | LQMNSLRAEDTAVYYCARRAGIIGTIGYYYGMDVWGQGTT |
| | Bispecific HLE | | | VTVSSGGGGSGGGGSGGGGSSYELTQPPSVSVSPGQTASIT |
| | molecule | | | CSGDRLGEKYTSWYQQRPGQSPLLVIYQDTKRPSGIPERFS |
| | | | | GSNSGNTATLTISGTQAMDEADYYCQAWESSTVVFGGGTK |
| | | | | LTVLSGGGGSEVQLVESGGGLVQPGGSLKLSCAASGFTFNK |
| | | | | YAMNWVRQAPGKGLEWVARIRSKYNNYATYYADSVKDRF |
| | | | | TISRDDSKNTAYLQMNNLKTEDTAVYYCVRHGNFGNSYISY |
| | | | | WAYWGQGTLVTVSSGGGGGGGGSGGGGSQTVVTQEPS |
| | | | | LTVSPGGTVTLTCGSSTGAVTSGNYPNWVQQKPGQAPRG |
| | | | | LIGGTKFLAPGTPARFSGSLLGGKAALTLSGVQPEDEAEYYC |
| | | | | VLWYSNRWVFGGGTKLTVLGGGGDKTHTCPPCPAPELLG |
| | | | | GPSVFLFPPKPKDTLMISRTPEVTCVVVDVSHEDPEVKFNW |
| | | | | YVDGVEVHNAKTKPCEEQYGSTYRCVSVLTVLHQDWLNGK |
| | | | | EYKCKVSNKALPAPIEKTISKAKGQPREPQVYTLPPSREEMT |
| | | | | KNQVSLTCLVKGFYPSDIAVEWESNGQPENNYKTTPPVLDS |
| | | | | DGSFFLYSKLTVDKSRWQQGNVFSCSVMHEALHNHYTQKS |
| | | | | LSLSPGGGGGGGGSGGGGSGGGGSGGGGSGGGGSDKT |
| | | | | HTCPPCPAPELLGGPSVFLFPPKPKDTLMISRTPEVTCVVVD |
| | | | | VSHEDPEVKFNWYVDGVEVHNAKTKPCEEQYGSTYRCVSV |
| | | | | LTVLHQDWLNGKEYKCKVSNKALPAPIEKTISKAKGQPREP |
| | | | | QVYTLPPSREEMTKNQVSLTCLVKGFYPSDIAVEWESNGQP |
| | | | | ENNYKTTPPVLDSDGSFFLYSKLTVDKSRWQQGNVFSCSV |
| | | | | MHEALHNHYTQKSLSLSPGK |
| 77. | CDH19 | artificial | aa | QVQLVESGGGVVQPGGSLRLSCAASGFTFSSYGMHWVRQ |
| | 65254.007_CC x | | | APGKCLEWVAFIWYEGSNKYYAESVKDRFTISRDNSKNTLYL |
| | I2C-scFc VH | | | QMNSLRAEDTAVYYCARRAGIIGTIGYYYGMDVWGQGTT |
| | | | | VTVSS |
| 78. | CDH19 | artificial | aa | SYELTQPPSVSVSPGQTASITCSGDRLGEKYTSWYQQRPGQ |
| | 65254.007_CC x | | | SPLLVIYQDTKRPSGIPERFSGSNSGNTATLTISGTQAMDEA |
| | I2C-scFc VL | | | DYYCQAWESSTVVFGCGTKLTVL |
| 79. | CDH19 | artificial | aa | QVQLVESGGGVVQPGGSLRLSCAASGFTFSSYGMHWVRQ |
| | 65254.007_CC x | | | APGKCLEWVAFIWYEGSNKYYAESVKDRFTISRDNSKNTLYL |
| | I2C-scFc scFv | | | QMNSLRAEDTAVYYCARRAGIIGTIGYYYGMDVWGQGTT |
| | | | | VTVSSGGGGSGGGGSGGGGSSYELTQPPSVSVSPGQTASIT |
| | | | | CSGDRLGEKYTSWYQQRPGQSPLLVIYQDTKRPSGIPERFS |
| | | | | GSNSGNTATLTISGTQAMDEADYYCQAWESSTVVFGCGTK |
| | | | | LTVL |
| 80. | CDH19 | artificial | aa | QVQLVESGGGVVQPGGSLRLSCAASGFTFSSYGMHWVRQ |
| | 65254.007_CC x | | | APGKCLEWVAFIWYEGSNKYYAESVKDRFTISRDNSKNTLYL |
| | I2C-scFc | | | QMNSLRAEDTAVYYCARRAGIIGTIGYYYGMDVWGQGTT |
| | Bispecific | | | VTVSSGGGGSGGGGSGGGGSSYELTQPPSVSVSPGQTASIT |
| | molecule | | | CSGDRLGEKYTSWYQQRPGQSPLLVIYQDTKRPSGIPERFS |
| | | | | GSNSGNTATLTISGTQAMDEADYYCQAWESSTVVFGCGTK |
| | | | | LTVLSGGGGSEVQLVESGGGLVQPGGSLKLSCAASGFTFNK |
| | | | | YAMNWVRQAPGKGLEWVARIRSKYNNYATYYADSVKDRF |
| | | | | TISRDDSKNTAYLQMNNLKTEDTAVYYCVRHGNFGNSYISY |
| | | | | WAYWGQGTLVTVSSGGGGSGGGGSGGGGSQTVVTQEPS |
| | | | | LTVSPGGTVTLTCGSSTGAVTSGNYPNWVQQKPGQAPRG |
| | | | | LIGGTKFLAPGTPARFSGSLLGGKAALTLSGVQPEDEAEYYC |
| | | | | VLWYSNRWVFGGGTKLTVL |
| 81. | CDH19 | artificial | aa | QVQLVESGGGVVQPGGSLRLSCAASGFTFSSYGMHWVRQ |
| | 65254.007_CC x | | | APGKCLEWVAFIWYEGSNKYYAESVKDRFTISRDNSKNTLYL |
| | I2C-scFc | | | QMNSLRAEDTAVYYCARRAGIIGTIGYYYGMDVWGQGTT |
| | Bispecific HLE | | | VTVSSGGGGSGGGGSGGGGSSYELTQPPSVSVSPGQTASIT |
| | molecule | | | CSGDRLGEKYTSWYQQRPGQSPLLVIYQDTKRPSGIPERFS |
| | | | | GSNSGNTATLTISGTQAMDEADYYCQAWESSTVVFGCGTK |
| | | | | LTVLSGGGGSEVQLVESGGGLVQPGGSLKLSCAASGFTFNK |
| | | | | YAMNWVRQAPGKGLEWVARIRSKYNNYATYYADSVKDRF |
| | | | | TISRDDSKNTAYLQMNNLKTEDTAVYYCVRHGNFGNSYISY |
| | | | | WAYWGQGTLVTVSSGGGGSGGGGSGGGGSQTVVTQEPS |
| | | | | LTVSPGGTVTLTCGSSTGAVTSGNYPNWVQQKPGQAPRG |
| | | | | LIGGTKFLAPGTPARFSGSLLGGKAALTLSGVQPEDEAEYYC |
| | | | | VLWYSNRWVFGGGTKLTVLGGGGDKTHTCPPCPAPELLG |
| | | | | GPSVFLFPPKPKDTLMISRTPEVTCVVVDVSHEDPEVKFNW |
| | | | | YVDGVEVHNAKTKPCEEQYGSTYRCVSVLTVLHQDWLNGK |
| | | | | EYKCKVSNKALPAPIEKTISKAKGQPREPQVYTLPPSREEMT |
| | | | | KNQVSLTCLVKGFYPSDIAVEWESNGQPENNYKTTPPVLDS |
| | | | | DGSFFLYSKLTVDKSRWQQGNVFSCSVMHEALHNHYTQKS |
| | | | | LSLSPGKGGGGSGGGGSGGGGSGGGGSGGGGSGGGGSD |
| | | | | KTHTCPPCPAPELLGGPSVFLFPPKPKDTLMISRTPEVTCVV |
| | | | | VDVSHEDPEVKFNWYVDGVEVHNAKTKPCEEQYGSTYRC |
| | | | | VSVLTVLHQDWLNGKEYKCKVSNKALPAPIEKTISKAKGQP |
| | | | | REPQVYTLPPSREEMTKNQVSLTCLVKGFYPSDIAVEWESN |
| | | | | GQPENNYKTTPPVLDSDGSFFLYSKLTVDKSRWQQGNVFS |
| | | | | CSVMHEALHNHYTQKSLSLSPGK |
| 82. | CDH19 | artificial | aa | QVQLVESGGGVVQPGGSLRLSCAASGFTFSSYGMHWVRQ |
| | 65254.007_CC x | | | APGKCLEWVAFIWYEGSNKYYAESVKDRFTISRDNSKNTLYL |
| | I2C-scFc_delGK | | | QMNSLRAEDTAVYYCARRAGIIGTIGYYYGMDVWGQGTT |
| | Bispecific HLE | | | VTVSSGGGGSGGGGSGGGGSSYELTQPPSVSVSPGQTASIT |
| | molecule | | | CSGDRLGEKYTSWYQQRPGQSPLLVIYQDTKRPSGIPERFS |
| | | | | GSNSGNTATLTISGTQAMDEADYYCQAWESSTVVFGCGTK |
| | | | | LTVLSGGGGSEVQLVESGGGLVQPGGSLKLSCAASGFTFNK |
| | | | | YAMNWVRQAPGKGLEWVARIRSKYNNYATYYADSVKDRF |
| | | | | TISRDDSKNTAYLQMNNLKTEDTAVYYCVRHGNFGNSYISY |
| | | | | WAYWGQGTLVTVSSGGGGSGGGGSGGGGSQTVVTQEPS |
| | | | | LTVSPGGTVTLTCGSSTGAVTSGNYPNWVQQKPGQAPRG |
| | | | | LIGGTKFLAPGTPARFSGSLLGGKAALTLSGVQPEDEAEYYC |
| | | | | VLWYSNRWVFGGGTKLTVLGGGGDKTHTCPPCPAPELLG |
| | | | | GPSVFLFPPKPKDTLMISRTPEVTCVVVDVSHEDPEVKFNW |
| | | | | YVDGVEVHNAKTKPCEEQYGSTYRCVSVLTVLHQDWLNGK |
| | | | | EYKCKVSNKALPAPIEKTISKAKGQPREPQVYTLPPSREEMT |
| | | | | KNQVSLTCLVKGFYPSDIAVEWESNGQPENNYKTTPPVLDS |
| | | | | DGSFFLYSKLTVDKSRWQQGNVFSCSVMHEALHNHYTQKS |
| | | | | LSLSPGGGGSGGGGSGGGGSGGGGSGGGGSGGGGSDKT |
| | | | | HTCPPCPAPELLGGPSVFLFPPKPKDTLMISRTPEVTCVVVD |
| | | | | VSHEDPEVKFNWYVDGVEVHNAKTKPCEEQYGSTYRCVSV |
| | | | | LTVLHQDWLNGKEYKCKVSNKALPAPIEKTISKAKGQPREP |
| | | | | QVYTLPPSREEMTKNQVSLTCLVKGFYPSDIAVEWESNGQP |
| | | | | ENNYKTTPPVLDSDGSFFLYSKLTVDKSRWQQGNVFSCSV |
| | | | | MHEALHNHYTQKSLSLSPGK |
| 83. | FLT3_7 A8xCD3- | artificial | aa | NARMGVS |
| | scFc VH CDR1 | | | |
| 84. | FLT3_7 A8xCD3- | artificial | aa | HIFSNDEKSYSTSLKN |
| | scFc VH CDR2 | | | |
| 85. | FLT3_7 A8xCD3- | artificial | aa | IVGYGSGWYGFFDY |
| | scFc VH CDR3 | | | |
| 86. | FLT3_7 A8xCD3- | artificial | aa | RASQGIRNDLG |
| | scFc VL CDR1 | | | |
| 87. | FLT3_7 A8xCD3- | artificial | aa | AASTLQS |
| | scFc VL CDR2 | | | |
| 88. | FLT3_7 A8xCD3- | artificial | aa | LQHNSYPLT |
| | scFc VL CDR3 | | | |
| 89. | FLT3_7 A8xCD3- | artificial | aa | QVTLKESGPTLVKPTETLTLTCTLSGFSLNNARMGVSWIRQ |
| | scFc VH | | | PPGKCLEWLAHIFSNDEKSYSTSLKNRLTISKDSSKTQVVLT |
| | | | | MTNVDPVDTATYYCARIVGYGSGWYGFFDYWGQGTLVTV |
| | | | | SS |
| 90. | FLT3_ A8-scFc VL | artificial | aa | DIQMTQSPSSLSASVGDRVTITCRASQGIRNDLGWYQQKP |
| | | | | GKAPKRLIYAASTLQSGVPSRFSGSGSGTEFTLTISSLQPEDF |
| | | | | ATYYCLQHNSYPLTFGCGTKVEIK |
| 91. | FLT3_7 A8xCD3- | artificial | aa | QVTLKESGPTLVKPTETLTLTCTLSGFSLNNARMGVSWIRQ |
| | scFv | | | PPGKCLEWLAHIFSNDEKSYSTSLKNRLTISKDSSKTQVVLT |
| | | | | MTNVDPVDTATYYCARIVGYGSGWYGFFDYWGQGTLVTV |
| | | | | SSGGGGSGGGGSGGGGSDIQMTQSPSSLSASVGDRVTITC |
| | | | | RASQGIRNDLGWYQQKPGKAPKRLIYAASTLQSGVPSRFSG |
| | | | | SGSGTEFTLTISSLQPEDFATYYCLQHNSYPLTFGCGTKVEIK |
| 92. | FLT3_7 A8xCD3 | artificial | aa | QVTLKESGPTLVKPTETLTLTCTLSGFSLNNARMGVSWIRQ |
| | Bispecific | | | PPGKCLEWLAHIFSNDEKSYSTSLKNRLTISKDSSKTQVVLT |
| | molecule | | | MTNVDPVDTATYYCARIVGYGSGWYGFFDYWGQGTLVTV |
| | | | | SSGGGGSGGGGSGGGGSDIQMTQSPSSLSASVGDRVTITC |
| | | | | RASQGIRNDLGWYQQKPGKAPKRLIYAASTLQSGVPSRFSG |
| | | | | SGSGTEFTLTISSLQPEDFATYYCLQHNSYPLTFGCGTKVEIK |
| | | | | SGGGGSEVQLVESGGGLVQPGGSLKLSCAASGFTFNKYAM |
| | | | | NWVRQAPGKGLEWVARIRSKYNNYATYYADSVKDRFTISR |
| | | | | DDSKNTAYLQMNNLKTEDTAVYYCVRHGNFGNSYISYWAY |
| | | | | WGQGTLVTVSSGGGGSGGGGSGGGGSQTVVTQEPSLTVS |
| | | | | PGGTVTLTCGSSTGAVTSGNYPNWVQQKPGQAPRGLIGG |
| | | | | TKFLAPGTPARFSGSLLGGKAALTLSGVQPEDEAEYYCVLW |
| | | | | YSNRWVFGGGTKLTVL |
| 93. | FLT3_7 A8xCD3- | artificial | aa | QVTLKESGPTLVKPTETLTLTCTLSGFSLNNARMGVSWIRQ |
| | scFc Bispecific | | | PPGKCLEWLAHIFSNDEKSYSTSLKNRLTISKDSSKTQVVLT |
| | HLE molecule | | | MTNVDPVDTATYYCARIVGYGSGWYGFFDYWGQGTLVTV |
| | | | | SSGGGGSGGGGSGGGGSDIQMTQSPSSLSASVGDRVTITC |
| | | | | RASQGIRNDLGWYQQKPGKAPKRLIYAASTLQSGVPSRFSG |
| | | | | SGSGTEFTLTISSLQPEDFATYYCLQHNSYPLTFGCGTKVEIK |
| | | | | SGGGGSEVQLVESGGGLVQPGGSLKLSCAASGFTFNKYAM |
| | | | | NWVRQAPGKGLEWVARIRSKYNNYATYYADSVKDRFTISR |
| | | | | DDSKNTAYLQMNNLKTEDTAVYYCVRHGNFGNSYISYWAY |
| | | | | WGQGTLVTVSSGGGGSGGGGSGGGGSQTVVTQEPSLTVS |
| | | | | PGGTVTLTCGSSTGAVTSGNYPNWVQQKPGQAPRGLIGG |
| | | | | TKFLAPGTPARFSGSLLGGKAALTLSGVQPEDEAEYYCVLW |
| | | | | YSNRWVFGGGTKLTVLGGGGDKTHTCPPCPAPELLGGPSV |
| | | | | FLFPPKPKDTLMISRTPEVTCVVVDVSHEDPEVKFNWYVDG |
| | | | | VEVHNAKTKPCEEQYGSTYRCVSVLTVLHQDWLNGKEYKC |
| | | | | KVSNKALPAPIEKTISKAKGQPREPQVYTLPPSREEMTKNQV |
| | | | | SLTCLVKGFYPSDIAVEWESNGQPENNYKTTPPVLDSDGSF |
| | | | | FLYSKLTVDKSRWQQGNVFSCSVMHEALHNHYTQKSLSLS |
| | | | | PGKGGGGSGGGGSGGGGSGGGGSGGGGSGGGGSDKTH |
| | | | | TCPPCPAPELLGGPSVFLFPPKPKDTLMISRTPEVTCVVVDV |
| | | | | SHEDPEVKFNWYVDGVEVHNAKTKPCEEQYGSTYRCVSVL |
| | | | | TVLHQDWLNGKEYKCKVSNKALPAPIEKTISKAKGQPREPQ |
| | | | | VYTLPPSREEMTKNQVSLTCLVKGFYPSDIAVEWESNGQPE |
| | | | | NNYKTTPPVLDSDGSFFLYSKLTVDKSRWQQGNVFSCSVM |
| | | | | HEALHNHYTQKSLSLSPGK |
| 94. | VH CDR1 | artificial | aa | SYYWS |
| | DLL3_1_CC_delG | | | |
| | K | | | |
| 95. | VH CDR2 | artificial | aa | YVYYSGTTNYNPSLKS |
| | DLL3_1_CC_delG | | | |
| | K | | | |
| 96. | VH CDR3 | artificial | aa | IAVTGFYFDY |
| | DLL3_1_CC_delG | | | |
| | K | | | |
| 97. | VL CDR1 | artificial | aa | RASQRVNNNYLA |
| | DLL3_1_CC_delG | | | |
| | K | | | |
| 98. | VL CDR2 | artificial | aa | GASSRAT |
| | DLL3_1_CC_delG | | | |
| | K | | | |
| 99. | VL CDR3 | artificial | aa | QQYDRSPLT |
| | DLL3_1_CC_delG | | | |
| | K | | | |
| 100. | VH | artificial | aa | QVQLQESGPGLVKPSETLSLTCTVSGGSISSYYWSWIRQPP |
| | DLL3_1_CC_delG | | | GKCLEWIGYVYYSGTTNYNPSLKSRVTISVDTSKNQFSLKLSS |
| | K | | | VTAADTAVYYCASIAVTGFYFDYWGQGTLVTVSS |
| 101. | VL | artificial | aa | EIVLTQSPGTLSLSPGERVTLSCRASQRVNNNYLAWYQQRP |
| | DLL3_1_CC_delG | | | GQAPRLLIYGASSRATGIPDRFSGSGSGTDFTLTISRLEPEDF |
| | K | | | AVYYCQQYDRSPLTFGCGTKLEIK |
| 102. | DLL3_1_CC_delG | artificial | aa | QVQLQESGPGLVKPSETLSLTCTVSGGSISSYYWSWIRQPP |
| | K | | | GKCLEWIGYVYYSGTTNYNPSLKSRVTISVDTSKNQFSLKLSS |
| | | | | VTAADTAVYYCASIAVTGFYFDYWGQGTLVTVSSGGGGSG |
| | | | | GGGSGGGGSEIVLTQSPGTLSLSPGERVTLSCRASQRVNNN |
| | | | | YLAWYQQRPGQAPRLLIYGASSRATGIPDRFSGSGSGTDFT |
| | | | | LTISRLEPEDFAVYYCQQYDRSPLTFGCGTKLEIK |
| 103. | DLL3_1_CCxCD3_ | artificial | aa | QVQLQESGPGLVKPSETLSLTCTVSGGSISSYYWSWIRQPP |
| | delGK Bispecific | | | GKCLEWIGYVYYSGTTNYNPSLKSRVTISVDTSKNQFSLKLSS |
| | molecule | | | VTAADTAVYYCASIAVTGFYFDYWGQGTLVTVSSGGGGSG |
| | | | | GGGSGGGGSEIVLTQSPGTLSLSPGERVTLSCRASQRVNNN |
| | | | | YLAWYQQRPGQAPRLLIYGASSRATGIPDRFSGSGSGTDFT |
| | | | | LTISRLEPEDFAVYYCQQYDRSPLTFGCGTKLEIKSGGGGSEV |
| | | | | QLVESGGGLVQPGGSLKLSCAASGFTFNKYAMNWVRQAP |
| | | | | GKGLEWVARIRSKYNNYATYYADSVKDRFTISRDDSKNTAY |
| | | | | LQMNNLKTEDTAVYYCVRHGNFGNSYISYWAYWGQGTLV |
| | | | | TVSSGGGGSGGGGSGGGGSQTVVTQEPSLTVSPGGTVTLT |
| | | | | CGSSTGAVTSGNYPNWVQQKPGQAPRGLIGGTKFLAPGTP |
| | | | | ARFSGSLLGGKAALTLSGVQPEDEAEYYCVLWYSNRWVFG |
| | | | | GGTKLTVL |
| | | | | QVQLQESGPGLVKPSETLSLTCTVSGGSISSYYWSWIRQPP |
| | | | | GKCLEWIGYVYYSGTTNYNPSLKSRVTISVDTSKNQFSLKLSS |
| | | | | VTAADTAVYYCASIAVTGFYFDYWGQGTLVTVSSGGGGSG |
| | | | | GGGSGGGGSEIVLTQSPGTLSLSPGERVTLSCRASQRVNNN |
| | | | | YLAWYQQRPGQAPRLLIYGASSRATGIPDRFSGSGSGTDFT |
| | | | | LTISRLEPEDFAVYYCQQYDRSPLTFGCGTKLEIKSGGGGSEV |
| 104. | DLL3_1_CCxCD3- | artificial | aa | QLVESGGGLVQPGGSLKLSCAASGFTFNKYAMNWVRQAP |
| | scFc_delGK | | | GKGLEWVARIRSKYNNYATYYADSVKDRFTISRDDSKNTAY |
| | Bispecific HLE | | | LQMNNLKTEDTAVYYCVRHGNFGNSYISYWAYWGQGTLV |
| | molecule | | | TVSSGGGGSGGGGSGGGGSQTVVTQEPSLTVSPGGTVTLT |
| | | | | CGSSTGAVTSGNYPNWVQQKPGQAPRGLIGGTKFLAPGTP |
| | | | | ARFSGSLLGGKAALTLSGVQPEDEAEYYCVLWYSNRWVFG |
| | | | | GGTKLTVLGGGGDKTHTCPPCPAPELLGGPSVFLFPPKPKD |
| | | | | TLMISRTPEVTCVVVDVSHEDPEVKFNWYVDGVEVHNAKT |
| | | | | KPCEEQYGSTYRCVSVLTVLHQDWLNGKEYKCKVSNKALPA |
| | | | | PIEKTISKAKGQPREPQVYTLPPSREEMTKNQVSLTCLVKGF |
| | | | | YPSDIAVEWESNGQPENNYKTTPPVLDSDGSFFLYSKLTVD |
| | | | | KSRWQQGNVFSCSVMHEALHNHYTQKSLSLSPGGGGSGG |
| | | | | GGSGGGGGGGGSGGGGSGGGGSDKTHTCPPCPAPELLG |
| | | | | GPSVFLFPPKPKDTLMISRTPEVTCVVVDVSHEDPEVKFNW |
| | | | | YVDGVEVHNAKTKPCEEQYGSTYRCVSVLTVLHQDWLNGK |
| | | | | EYKCKVSNKALPAPIEKTISKAKGQPREPQVYTLPPSREEMT |
| | | | | KNQVSLTCLVKGFYPSDIAVEWESNGQPENNYKTTPPVLDS |
| | | | | DGSFFLYSKLTVDKSRWQQGNVFSCSVMHEALHNHYTQKS |
| | | | | LSLSPGK |
| 105. | VH CDR1 CD19 | artificial | aa | SYGMH |
| | 97-G1RE-C2 | | | |
| 106. | VH CDR2 CD19 | artificial | aa | VISYEGSNKYYAESVKG |
| | 97-G1RE-C2 | | | |
| 107. | VH CDR3 CD19 | artificial | aa | DRGTIFGNYGLEV |
| | 97-G1RE-C2 | | | |
| 108. | VH CD19 97- | artificial | aa | QVQLVESGGGVVQPGRSLRLSCAASGFTFSSYGMHWVRQ |
| | G1RE-C2 CC | | | APGKCLEWVAVISYEGSNKYYAESVKGRFTISRDNSKNTLYL |
| | | | | QMNSLRDEDTAVYYCARDRGTIFGNYGLEVWGQGTTVTV |
| | | | | SS |
| 109. | VL CDR1 CD19 | artificial | aa | RSSQSLLHKNAFNYLD |
| | 97-G1RE-C2 | | | |
| 110. | VL CDR2 CD19 | artificial | aa | LGSNRAS |
| | 97-G1RE-C2 | | | |
| 111. | VL CDR3 CD19 | artificial | aa | MQALQTPFT |
| | 97-G1RE-C2 | | | |
| 112. | VL CD19 97- | artificial | aa | DIVMTQSPLSLPVISGEPASISCRSSQSLLHKNAFNYLDWYL |
| | G1RE-C2 CC | | | QKPGQSPQLLIYLGSNRASGVPDRFSGSGSGTDFTLKISRVE |
| | | | | AEDVGVYYCMQALQTPFTFGCGTKVDIK |
| 113. | CD19 97-G1RE- | artificial | aa | MDMRVPAQLLGLLLLWLRGARCDIVMTQSPLSLPVISGEP |
| | C2 CC x I2C0 | | | ASISCRSSQSLLHKNAFNYLDWYLQKPGQSPQLLIYLGSNRA |
| | | | | SGVPDRFSGSGSGTDFTLKISRVEAEDVGVYYCMQALQTPF |
| | | | | TFGCGTKVDIKGGGGSGGGGSGGGGSQVQLVESGGGVVQ |
| | | | | PGRSLRLSCAASGFTFSSYGMHWVRQAPGKCLEWVAVISY |
| | | | | EGSNKYYAESVKGRFTISRDNSKNTLYLQMNSLRDEDTAVY |
| | | | | YCARDRGTIFGNYGLEVWGQGTTVTVSSGGGGSEVQLVES |
| | | | | GGGLVQPGGSLKLSCAASGFTFNKYAMNWVRQAPGKGLE |
| | | | | WVARIRSKYNNYATYYADSVKDRFTISRDDSKNTAYLQMN |
| | | | | NLKTEDTAVYYCVRHGNFGNSYISYWAYWGQGTLVTVSSG |
| | | | | GGGSGGGGSGGGGSQTVVTQEPSLTVSPGGTVTLTCGSST |
| | | | | GAVTSGNYPNWVQQKPGQAPRGLIGGTKFLAPGTPARFS |
| | | | | GSLLGGKAALTLSGVQPEDEAEYYCVLWYSNRWVFGGGTK |
| | | | | LTVL |
| 114. | CD19 97-G1RE- | artificial | aa | MDMRVPAQLLGLLLLWLRGARCDIVMTQSPLSLPVISGEP |
| | C2 CC x 12C0 | | | ASISCRSSQSLLHKNAFNYLDWYLQKPGQSPQLLIYLGSNRA |
| | | | | SGVPDRFSGSGSGTDFTLKISRVEAEDVGVYYCMQALQTPF |
| | | | | TFGCGTKVDIKGGGGSGGGGSGGGGSQVQLVESGGGVVQ |
| | | | | PGRSLRLSCAASGFTFSSYGMHWVRQAPGKCLEWVAVISY |
| | | | | EGSNKYYAESVKGRFTISRDNSKNTLYLQMNSLRDEDTAVY |
| | | | | YCARDRGTIFGNYGLEVWGQGTTVTVSSGGGGSEVQLVES |
| | | | | GGGLVQPGGSLKLSCAASGFTFNKYAMNWVRQAPGKGLE |
| | | | | WVARIRSKYNNYATYYADSVKDRFTISRDDSKNTAYLQMN |
| | | | | NLKTEDTAVYYCVRHGNFGNSYISYWAYWGQGTLVTVSSG |
| | | | | GGGSGGGGSGGGGSQTVVTQEPSLTVSPGGTVTLTCGSST |
| | | | | GAVTSGNYPNWVQQKPGQAPRGLIGGTKFLAPGTPARFS |
| | | | | GSLLGGKAALTLSGVQPEDEAEYYCVLWYSNRWVFGGGTK |
| | | | | LTVLGGGGDKTHTCPPCPAPELLGGPSVFLFPPKPKDTLMIS |
| | | | | RTPEVTCVVVDVSHEDPEVKFNWYVDGVEVHNAKTKPCEE |
| | | | | QYGSTYRCVSVLTVLHQDWLNGKEYKCKVSNKALPAPIEKTI |
| | | | | SKAKGQPREPQVYTLPPSREEMTKNQVSLTCLVKGFYPSDI |
| | | | | AVEWESNGQPENNYKTTPPVLDSDGSFFLYSKLTVDKSRW |
| | | | | QQGNVFSCSVMHEALHNHYTQKSLSLSPGKGGGGSGGGG |
| | | | | SGGGGSGGGGSGGGGSGGGGSDKTHTCPPCPAPELLGGP |
| | | | | SVFLFPPKPKDTLMISRTPEVTCVVVDVSHEDPEVKFNWYV |
| | | | | DGVEVHNAKTKPCEEQYGSTYRCVSVLTVLHQDWLNGKEY |
| | | | | KCKVSNKALPAPIEKTISKAKGQPREPQVYTLPPSREEMTKN |
| | | | | QVSLTCLVKGFYPSDIAVEWESNGQPENNYKTTPPVLDSDG |
| | | | | SFFLYSKLTVDKSRWQQGNVFSCSVMHEALHNHYTQKSLS |
| | | | | LSPGK |
| 115. | VH CDR1 CDH3 | artificial | aa | SYPIN |
| | G8A 6-B12 | | | |
| 116. | VH CDR2 CDH3 | artificial | aa | VIWTGGGTNYASSVKG |
| | G8A 6-B12 | | | |
| 117. | VH CDR3 CDH3 | artificial | aa | SRGVYDFDGRGAMDY |
| | G8A 6-B12 | | | |
| 118. | VL CDR1 CDH3 | artificial | aa | KSSQSLLYSSNQKNYFA |
| | G8A 6-B12 | | | |
| 119. | VL CDR2 CDH3 | artificial | aa | WASTRES |
| | G8A 6-B12 | | | |
| 120. | VL CDR3 CDH3 | artificial | aa | QQYYSYPYT |
| | G8A 6-B12 | | | |
| 121. | VH CDH3 G8A 6- | artificial | aa | EVQLLESGGGLVQPGGSLRLSCAASGFSFSSYPINWVRQAP |
| | B12 | | | GKGLEWVGVIWTGGGTNYASSVKGRFTISRDNSKNTVYLQ |
| | | | | MNSLRAEDTAVYYCAKSRGVYDFDGRGAMDYWGQGTLV |
| | | | | TVSS |
| 122. | VL CDH3 G8A 6- | artificial | aa | DIVMTQSPDSLAVSLGERATINCKSSQSLLYSSNQKNYFAW |
| | B12 | | | YQQKPGQPPKLLIYWASTRESGVPDRFSGSGSGTDFTLTISS |
| | | | | LQAEDVAVYYCQQYYSYPYTFGQGTKLEIK |
| 123. | CDH3 G8A 6-B12 | artificial | aa | EVQLLESGGGLVQPGGSLRLSCAASGFSFSSYPINWVRQAP |
| | scFv | | | GKGLEWVGVIWTGGGTNYASSVKGRFTISRDNSKNTVYLQ |
| | | | | MNSLRAEDTAVYYCAKSRGVYDFDGRGAMDYWGQGTLV |
| | | | | TVSSGGGGSGGGGSGGGGSDIVMTQSPDSLAVSLGERATI |
| | | | | NCKSSQSLLYSSNQKNYFAWYQQKPGQPPKLLIYWASTRES |
| | | | | GVPDRFSGSGSGTDFTLTISSLQAEDVAVYYCQQYYSYPYTF |
| | | | | GQGTKLEIK |
| 124. | CDH3 G8A 6-B12 | artificial | aa | EVQLLESGGGLVQPGGSLRLSCAASGFSFSSYPINWVRQAP |
| | x I2C0 bispecific | | | GKGLEWVGVIWTGGGTNYASSVKGRFTISRDNSKNTVYLQ |
| | molecule | | | MNSLRAEDTAVYYCAKSRGVYDFDGRGAMDYWGQGTLV |
| | | | | TVSSGGGGSGGGGSGGGGSDIVMTQSPDSLAVSLGERATI |
| | | | | NCKSSQSLLYSSNQKNYFAWYQQKPGQPPKLLIYWASTRES |
| | | | | GVPDRFSGSGSGTDFTLTISSLQAEDVAVYYCQQYYSYPYTF |
| | | | | GQGTKLEIKSGGGGSEVQLVESGGGLVQPGGSLKLSCAASG |
| | | | | FTFNKYAMNWVRQAPGKGLEWVARIRSKYNNYATYYADS |
| | | | | VKDRFTISRDDSKNTAYLQMNNLKTEDTAVYYCVRHGNFG |
| | | | | NSYISYWAYWGQGTLVTVSSGGGGSGGGGSGGGGSQTV |
| | | | | VTQEPSLTVSPGGTVTLTCGSSTGAVTSGNYPNWVQQKPG |
| | | | | QAPRGLIGGTKFLAPGTPARFSGSLLGGKAALTLSGVQPEDE |
| | | | | AEYYCVLWYSNRWVFGGGTKLTVL |
| 125. | CDH3 G8A 6-B12 | artificial | aa | EVQLLESGGGLVQPGGSLRLSCAASGFSFSSYPINWVRQAP |
| | x I2C0 bispecific | | | GKGLEWVGVIWTGGGTNYASSVKGRFTISRDNSKNTVYLQ |
| | molecule HLE | | | MNSLRAEDTAVYYCAKSRGVYDFDGRGAMDYWGQGTLV |
| | | | | TVSSGGGGSGGGGSGGGGSDIVMTQSPDSLAVSLGERATI |
| | | | | NCKSSQSLLYSSNQKNYFAWYQQKPGQPPKLLIYWASTRES |
| | | | | GVPDRFSGSGSGTDFTLTISSLQAEDVAVYYCQQYYSYPYTF |
| | | | | GQGTKLEIKSGGGGSEVQLVESGGGLVQPGGSLKLSCAASG |
| | | | | FTFNKYAMNWVRQAPGKGLEWVARIRSKYNNYATYYADS |
| | | | | VKDRFTISRDDSKNTAYLQMNNLKTEDTAVYYCVRHGNFG |
| | | | | NSYISYWAYWGQGTLVTVSSGGGGSGGGGSGGGGSQTV |
| | | | | VTQEPSLTVSPGGTVTLTCGSSTGAVTSGNYPNWVQQKPG |
| | | | | QAPRGLIGGTKFLAPGTPARFSGSLLGGKAALTLSGVQPEDE |
| | | | | AEYYCVLWYSNRWVFGGGTKLTVLGGGGDKTHTCPPCPA |
| | | | | PELLGGPSVFLFPPKPKDTLMISRTPEVTCVVVDVSHEDPEV |
| | | | | KFNWYVDGVEVHNAKTKPCEEQYGSTYRCVSVLTVLHQD |
| | | | | WLNGKEYKCKVSNKALPAPIEKTISKAKGQPREPQVYTLPPS |
| | | | | REEMTKNQVSLTCLVKGFYPSDIAVEWESNGQPENNYKTT |
| | | | | PPVLDSDGSFFLYSKLTVDKSRWQQGNVFSCSVMHEALHN |
| | | | | HYTQKSLSLSPGKGGGGSGGGGSGGGGSGGGGSGGGGS |
| | | | | GGGGSDKTHTCPPCPAPELLGGPSVFLFPPKPKDTLMISRTP |
| | | | | EVTCVVVDVSHEDPEVKFNWYVDGVEVHNAKTKPCEEQY |
| | | | | GSTYRCVSVLTVLHQDWLNGKEYKCKVSNKALPAPIEKTISK |
| | | | | AKGQPREPQVYTLPPSREEMTKNQVSLTCLVKGFYPSDIAV |
| | | | | EWESNGQPENNYKTTPPVLDSDGSFFLYSKLTVDKSRWQQ |
| | | | | GNVFSCSVMHEALHNHYTQKSLSLSPGK |
| 126. | BCMA A7 27-C4- | artificial | aa | NHIIH |
| | G7 CDR1 VH | | | |
| 127. | BCMA A7 27-C4- | artificial | aa | YINPYPGYHAYNEKFQG |
| | G7 CDR2 VH | | | |
| 128. | BCMA A7 27-C4- | artificial | aa | DGYYRDTDVLDY |
| | G7 CDR3 VH | | | |
| 129. | BCMA A7 27-C4- | artificial | aa | QASQDISNYLN |
| | G7 CDR1 VL | | | |
| 130. | BCMA A7 27-C4- | artificial | aa | YTSRLHT |
| | G7 CDR2 VL | | | |
| 131. | BCMA A7 27-C4- | artificial | aa | QQGNTLPWT |
| | G7 CDR3 VL | | | |
| 132. | BCMA A7 27-C4- | artificial | aa | QVQLVQSGAEVKKPGASVKVSCKASGYTFTNHIIHWVRQA |
| | G7 CC (44/100) | | | PGQCLEWMGYINPYPGYHAYNEKFQGRATMTSDTSTSTV |
| | VH | | | YMELSSLRSEDTAVYYCARDGYYRDTDVLDYWGQGTLVTV |
| | | | | SS |
| 133. | BCMA A7 27-C4- | artificial | aa | DIQMTQSPSSLSASVGDRVTITCQASQDISNYLNWYQQKP |
| | G7 CC (44/100) | | | GKAPKLLIYYTSRLHTGVPSRFSGSGSGTDFTFTISSLEPEDIA |
| | VL | | | TYYCQQGNTLPWTFGCGTKLEIK |
| 134. | BCMA A7 27-C4- | artificial | aa | QVQLVQSGAEVKKPGASVKVSCKASGYTFTNHIIHWVRQA |
| | G7 CC (44/100) | | | PGQCLEWMGYINPYPGYHAYNEKFQGRATMTSDTSTSTV |
| | scFv | | | YMELSSLRSEDTAVYYCARDGYYRDTDVLDYWGQGTLVTV |
| | | | | SSGGGGGGGGSGGGGSDIQMTQSPSSLSASVGDRVTITC |
| | | | | QASQDISNYLNWYQQKPGKAPKLLIYYTSRLHTGVPSRFSG |
| | | | | SGSGTDFTFTISSLEPEDIATYYCQQGNTLPWTFGCGTKLEIK |
| 135. | BCMA A7 27-C4- | artificial | aa | QVQLVQSGAEVKKPGASVKVSCKASGYTFTNHIIHWVRQA |
| | G7 CC (44/100) x | | | PGQCLEWMGYINPYPGYHAYNEKFQGRATMTSDTSTSTV |
| | I2C0 bispecific | | | YMELSSLRSEDTAVYYCARDGYYRDTDVLDYWGQGTLVTV |
| | molecule | | | SSGGGGSGGGGSGGGGSDIQMTQSPSSLSASVGDRVTITC |
| | | | | QASQDISNYLNWYQQKPGKAPKLLIYYTSRLHTGVPSRFSG |
| | | | | SGSGTDFTFTISSLEPEDIATYYCQQGNTLPWTFGCGTKLEIK |
| | | | | SGGGGSEVQLVESGGGLVQPGGSLKLSCAASGFTFNKYAM |
| | | | | NWVRQAPGKGLEWVARIRSKYNNYATYYADSVKDRFTISR |
| | | | | DDSKNTAYLQMNNLKTEDTAVYYCVRHGNFGNSYISYWAY |
| | | | | WGQGTLVTVSSGGGGSGGGGSGGGGSQTVVTQEPSLTVS |
| | | | | PGGTVTLTCGSSTGAVTSGNYPNWVQQKPGQAPRGLIGG |
| | | | | TKFLAPGTPARFSGSLLGGKAALTLSGVQPEDEAEYYCVLW |
| | | | | YSNRWVFGGGTKLTVL |
| 136. | BCMA A7 27-C4- | artificial | aa | QVQLVQSGAEVKKPGASVKVSCKASGYTFTNHIIHWVRQA |
| | G7 CC (44/100) x | | | PGQCLEWMGYINPYPGYHAYNEKFQGRATMTSDTSTSTV |
| | I2C0-scFc | | | YMELSSLRSEDTAVYYCARDGYYRDTDVLDYWGQGTLVTV |
| | bispecific | | | SSGGGGSGGGGSGGGGSDIQMTQSPSSLSASVGDRVTITC |
| | molecule HLE | | | QASQDISNYLNWYQQKPGKAPKLLIYYTSRLHTGVPSRFSG |
| | | | | SGSGTDFTFTISSLEPEDIATYYCQQGNTLPWTFGCGTKVEI |
| | | | | KSGGGGSEVQLVESGGGLVQPGGSLKLSCAASGFTFNKYA |
| | | | | MNWVRQAPGKGLEWVARIRSKYNNYATYYADSVKDRFTIS |
| | | | | RDDSKNTAYLQMNNLKTEDTAVYYCVRHGNFGNSYISYW |
| | | | | AYWGQGTLVTVSSGGGGSGGGGSGGGGSQTVVTQEPSLT |
| | | | | VSPGGTVTLTCGSSTGAVTSGNYPNWVQQKPGQAPRGLIG |
| | | | | GTKFLAPGTPARFSGSLLGGKAALTLSGVQPEDEAEYYCVL |
| | | | | WYSNRWVFGGGTKLTVLGGGGDKTHTCPPCPAPELLGGP |
| | | | | SVFLFPPKPKDTLMISRTPEVTCVVVDVSHEDPEVKFNWYV |
| | | | | DGVEVHNAKTKPCEEQYGSTYRCVSVLTVLHQDWLNGKEY |
| | | | | KCKVSNKALPAPIEKTISKAKGQPREPQVYTLPPSREEMTKN |
| | | | | QVSLTCLVKGFYPSDIAVEWESNGQPENNYKTTPPVLDSDG |
| | | | | SFFLYSKLTVDKSRWQQGNVFSCSVMHEALHNHYTQKSLS |
| | | | | LSPGKGGGGSGGGGSGGGGSGGGGSGGGGSGGGGSDKT |
| | | | | HTCPPCPAPELLGGPSVFLFPPKPKDTLMISRTPEVTCVVVD |
| | | | | VSHEDPEVKFNWYVDGVEVHNAKTKPCEEQYGSTYRCVSV |
| | | | | LTVLHQDWLNGKEYKCKVSNKALPAPIEKTISKAKGQPREP |
| | | | | QVYTLPPSREEMTKNQVSLTCLVKGFYPSDIAVEWESNGQP |
| | | | | ENNYKTTPPVLDSDGSFFLYSKLTVDKSRWQQGNVFSCSV |
| | | | | MHEALHNHYTQKSLSLSPGK |
| 137. | PM 76-B10.17 CC | artificial | aa | DYYMY |
| | VH CDR1 | | | |
| 138. | PM 76-B10.17 CC | artificial | aa | IISDAGYYTYYSDIIKG |
| | VH CDR2 | | | |
| 139. | PM 76-B10.17 CC | artificial | aa | GFPLLRHGAMDY |
| | VH CDR3 | | | |
| 140. | PM 76-B10.17 CC | artificial | aa | KASQNVDANVA |
| | VL CDR1 | | | |
| 141. | PM 76-B10.17 CC | artificial | aa | SASYVYW |
| | VL CDR2 | | | |
| 142. | PM 76-B10.17 CC | artificial | aa | QQYDQQLIT |
| | VL CDR3 | | | |
| 143. | PM 76-B10.17 CC | artificial | aa | QVQLVESGGGLVKPGESLRLSCAASGFTFSDYYMYWVRQA |
| | VH | | | PGKCLEWVAIISDAGYYTYYSDIIKGRFTISRDNAKNSLYLQM |
| | | | | NSLKAEDTAVYYCARGFPLLRHGAMDYWGQGTLVTVSS |
| 144. | PM 76-B10.17 CC | artificial | aa | DIQMTQSPSSLSASVGDRVTITCKASQNVDANVAWYQQK |
| | VL | | | PGQAPKSLIYSASYVYWDVPSRFSGSASGTDFTLTISSVQSE |
| | | | | DFATYYCQQYDQQLITFGCGTKLEIK |
| 145. | PM 76-B10.17 CC | artificial | aa | QVQLVESGGGLVKPGESLRLSCAASGFTFSDYYMYWVRQA |
| | x I2C0 bispecific | | | PGKCLEWVAIISDAGYYTYYSDIIKGRFTISRDNAKNSLYLQM |
| | molecule | | | NSLKAEDTAVYYCARGFPLLRHGAMDYWGQGTLVTVSSG |
| | | | | GGGSGGGGSGGGGSDIQMTQSPSSLSASVGDRVTITCKAS |
| | | | | QNVDANVAWYQQKPGQAPKSLIYSASYVYWDVPSRFSGS |
| | | | | ASGTDFTLTISSVQSEDFATYYCQQYDQQLITFGCGTKLEIK |
| 146. | PM 76-B10.17 CC | artificial | aa | QVQLVESGGGLVKPGESLRLSCAASGFTFSDYYMYWVRQA |
| | | | | PGKCLEWVAIISDAGYYTYYSDIIKGRFTISRDNAKNSLYLQM |
| | | | | NSLKAEDTAVYYCARGFPLLRHGAMDYWGQGTLVTVSSG |
| | | | | GGGSGGGGSGGGGSDIQMTQSPSSLSASVGDRVTITCKAS |
| | | | | QNVDANVAWYQQKPGQAPKSLIYSASYVYWDVPSRFSGS |
| | | | | ASGTDFTLTISSVQSEDFATYYCQQYDQQLITFGCGTKLEIKS |
| | | | | GGGGSEVQLVESGGGLVQPGGSLKLSCAASGFTFNKYAMN |
| | | | | WVRQAPGKGLEWVARIRSKYNNYATYYADSVKDRFTISRD |
| | | | | DSKNTAYLQMNNLKTEDTAVYYCVRHGNFGNSYISYWAY |
| | | | | WGQGTLVTVSSGGGGSGGGGSGGGGSQTVVTQEPSLTVS |
| | | | | PGGTVTLTCGSSTGAVTSGNYPNWVQQKPGQAPRGLIGG |
| | | | | TKFLAPGTPARFSGSLLGGKAALTLSGVQPEDEAEYYCVLW |
| | | | | YSNRWVFGGGTKLTVL |
| 147. | pm 76-B0.17 CC | artificial | aa | QVQLVESGGGLVKPGESLRLSCAASGFTFSDYYMYWVRQA |
| | x I2C0-scFc | | | PGKCLEWVAIISDAGYYTYYSDIIKGRFTISRDNAKNSLYLQM |
| | bispecific | | | NSLKAEDTAVYYCARGFPLLRHGAMDYWGQGTLVTVSSG |
| | HLE molecule | | | GGGSGGGGSGGGGSDIQMTQSPSSLSASVGDRVTITCKAS |
| | | | | QNVDANVAWYQQKPGQAPKSLIYSASYVYWDVPSRFSGS |
| | | | | ASGTDFTLTISSVQSEDFATYYCQQYDQQLITFGCGTKLEIKS |
| | | | | GGGGSEVQLVESGGGLVQPGGSLKLSCAASGFTFNKYAMN |
| | | | | WVRQAPGKGLEWVARIRSKYNNYATYYADSVKDRFTISRD |
| | | | | DSKNTAYLQMNNLKTEDTAVYYCVRHGNFGNSYISYWAY |
| | | | | WGQGTLVTVSSGGGGSGGGGSGGGGSQTVVTQEPSLTVS |
| | | | | PGGTVTLTCGSSTGAVTSGNYPNWVQQKPGQAPRGLIGG |
| | | | | TKFLAPGTPARFSGSLLGGKAALTLSGVQPEDEAEYYCVLW |
| | | | | YSNRWVFGGGTKLTVLGGGGDKTHTCPPCPAPELLGGPSV |
| | | | | FLFPPKPKDTLMISRTPEVTCVVVDVSHEDPEVKFNWYVDG |
| | | | | VEVHNAKTKPCEEQYGSTYRCVSVLTVLHQDWLNGKEYKC |
| | | | | KVSNKALPAPIEKTISKAKGQPREPQVYTLPPSREEMTKNQV |
| | | | | SLTCLVKGFYPSDIAVEWESNGQPENNYKTTPPVLDSDGSF |
| | | | | FLYSKLTVDKSRWQQGNVFSCSVMHEALHNHYTQKSLSLS |
| | | | | PGKGGGGSGGGGSGGGGSGGGGSGGGGSGGGGSDKTH |
| | | | | TCPPCPAPELLGGPSVFLFPPKPKDTLMISRTPEVTCVVVDV |
| | | | | SHEDPEVKFNWYVDGVEVHNAKTKPCEEQYGSTYRCVSVL |
| | | | | TVLHQDWLNGKEYKCKVSNKALPAPIEKTISKAKGQPREPQ |
| | | | | VYTLPPSREEMTKNQVSLTCLVKGFYPSDIAVEWESNGQPE |
| | | | | NNYKTTPPVLDSDGSFFLYSKLTVDKSRWQQGNVFSCSVM |
| | | | | HEALHNHYTQKSLSLSPGK |
| 148. | PM 76-B10.17 CC | artificial | aa | QVQLVESGGGLVKPGESLRLSCAASGFTFSDYYMYWVRQA |
| | × I2C0- | | | PGKCLEWVAIISDAGYYTYYSDIIKGRFTISRDNAKNSLYLQM |
| | scFc_delGK | | | NSLKAEDTAVYYCARGFPLLRHGAMDYWGQGTLVTVSSG |
| | bispecific | | | GGGSGGGGSGGGGSDIQMTQSPSSLSASVGDRVTITCKAS |
| | HLE molecule | | | QNVDANVAWYQQKPGQAPKSLIYSASYVYWDVPSRFSGS |
| | | | | ASGTDFTLTISSVQSEDFATYYCQQYDQQLITFGCGTKLEIKS |
| | | | | GGGGSEVQLVESGGGLVQPGGSLKLSCAASGFTFNKYAMN |
| | | | | WVRQAPGKGLEWVARIRSKYNNYATYYADSVKDRFTISRD |
| | | | | DSKNTAYLQMNNLKTEDTAVYYCVRHGNFGNSYISYWAY |
| | | | | WGQGTLVTVSSGGGGSGGGGSGGGGSQTVVTQEPSLTVS |
| | | | | PGGTVTLTCGSSTGAVTSGNYPNWVQQKPGQAPRGLIGG |
| | | | | TKFLAPGTPARFSGSLLGGKAALTLSGVQPEDEAEYYCVLW |
| | | | | YSNRWVFGGGTKLTVLGGGGDKTHTCPPCPAPELLGGPSV |
| | | | | FLFPPKPKDTLMISRTPEVTCVVVDVSHEDPEVKFNWYVDG |
| | | | | VEVHNAKTKPCEEQYGSTYRCVSVLTVLHQDWLNGKEYKC |
| | | | | KVSNKALPAPIEKTISKAKGQPREPQVYTLPPSREEMTKNQV |
| | | | | SLTCLVKGFYPSDIAVEWESNGQPENNYKTTPPVLDSDGSF |
| | | | | FLYSKLTVDKSRWQQGNVFSCSVMHEALHNHYTQKSLSLS |
| | | | | PGGGGGGGGSGGGGSGGGGSGGGGSGGGGSDKTHTCP |
| | | | | PCPAPELLGGPSVFLFPPKPKDTLMISRTPEVTCVVVDVSHE |
| | | | | DPEVKFNWYVDGVEVHNAKTKPCEEQYGSTYRCVSVLTVL |
| | | | | HQDWLNGKEYKCKVSNKALPAPIEKTISKAKGQPREPQVYT |
| | | | | LPPSREEMTKNQVSLTCLVKGFYPSDIAVEWESNGQPENN |
| | | | | YKTTPPVLDSDGSFFLYSKLTVDKSRWQQGNVFSCSVMHE |
| | | | | ALHNHYTQKSLSLSPGK |
| 149. | PM 76-B10.17 CC | artificial | aa | QVQLVESGGGLVKPGESLRLSCAASGFTFSDYYMYWVRQA |
| | × I2C0 CC | | | PGKCLEWVAIISDAGYYTYYSDIIKGRFTISRDNAKNSLYLQM |
| | (103/43)-scFc | | | NSLKAEDTAVYYCARGFPLLRHGAMDYWGQGTLVTVSSG |
| | bispecific | | | GGGSGGGGSGGGGSDIQMTQSPSSLSASVGDRVTITCKAS |
| | molecule | | | QNVDANVAWYQQKPGQAPKSLIYSASYVYWDVPSRFSGS |
| | | | | ASGTDFTLTISSVQSEDFATYYCQQYDQQLITFGCGTKLEIKS |
| | | | | GGGGSEVQLVESGGGLVQPGGSLKLSCAASGFTFNKYAMN |
| | | | | WVRQAPGKGLEWVARIRSKYNNYATYYADSVKDRFTISRD |
| | | | | DSKNTAYLQMNNLKTEDTAVYYCVRHGNFGNSYISYWAYC |
| | | | | GQGTLVTVSSGGGGSGGGGSGGGGSQTVVTQEPSLTVSP |
| | | | | GGTVTLTCGSSTGAVTSGNYPNWVQQKPGQCPRGLIGGT |
| | | | | KFLAPGTPARFSGSLLGGKAALTLSGVQPEDEAEYYCVLWY |
| | | | | SNRWVFGGGTKLTVL |
| 150. | PM 76-B10.17 CC | artificial | aa | QVQLVESGGGLVKPGESLRLSCAASGFTFSDYYMYWVRQA |
| | x I2C0 CC | | | PGKCLEWVAIISDAGYYTYYSDIIKGRFTISRDNAKNSLYLQM |
| | (103/43)-scFc | | | NSLKAEDTAVYYCARGFPLLRHGAMDYWGQGTLVTVSSG |
| | bispecific | | | GGGSGGGGSGGGGSDIQMTQSPSSLSASVGDRVTITCKAS |
| | HLE molecule | | | QNVDANVAWYQQKPGQAPKSLIYSASYVYWDVPSRFSGS |
| | | | | ASGTDFTLTISSVQSEDFATYYCQQYDQQLITFGCGTKLEIKS |
| | | | | GGGGSEVQLVESGGGLVQPGGSLKLSCAASGFTFNKYAMN |
| | | | | WVRQAPGKGLEWVARIRSKYNNYATYYADSVKDRFTISRD |
| | | | | DSKNTAYLQMNNLKTEDTAVYYCVRHGNFGNSYISYWAYC |
| | | | | GQGTLVTVSSGGGGSGGGGSGGGGSQTVVTQEPSLTVSP |
| | | | | GGTVTLTCGSSTGAVTSGNYPNWVQQKPGQCPRGLIGGT |
| | | | | KFLAPGTPARFSGSLLGGKAALTLSGVQPEDEAEYYCVLWY |
| | | | | SNRWVFGGGTKLTVLGGGGDKTHTCPPCPAPELLGGPSVF |
| | | | | LFPPKPKDTLMISRTPEVTCVVVDVSHEDPEVKFNWYVDG |
| | | | | VEVHNAKTKPCEEQYGSTYRCVSVLTVLHQDWLNGKEYKC |
| | | | | KVSNKALPAPIEKTISKAKGQPREPQVYTLPPSREEMTKNQV |
| | | | | SLTCLVKGFYPSDIAVEWESNGQPENNYKTTPPVLDSDGSF |
| | | | | FLYSKLTVDKSRWQQGNVFSCSVMHEALHNHYTQKSLSLS |
| | | | | PGKGGGGSGGGGSGGGGSGGGGSGGGGSGGGGSDKTH |
| | | | | TCPPCPAPELLGGPSVFLFPPKPKDTLMISRTPEVTCVVVDV |
| | | | | SHEDPEVKFNWYVDGVEVHNAKTKPCEEQYGSTYRCVSVL |
| | | | | TVLHQDWLNGKEYKCKVSNKALPAPIEKTISKAKGQPREPQ |
| | | | | VYTLPPSREEMTKNQVSLTCLVKGFYPSDIAVEWESNGQPE |
| | | | | NNYKTTPPVLDSDGSFFLYSKLTVDKSRWQQGNVFSCSVM |
| | | | | HEALHNHYTQKSLSLSPGK |
| 151. | PM 76-B10.17 CC | artificial | aa | QVQLVESGGGLVKPGESLRLSCAASGFTFSDYYMYWVRQA |
| | x I2C0 CC | | | PGKCLEWVAIISDAGYYTYYSDIIKGRFTISRDNAKNSLYLQM |
| | (103/43)-scFc | | | NSLKAEDTAVYYCARGFPLLRHGAMDYWGQGTLVTVSSG |
| | delGK bispecific | | | GGGSGGGGSGGGGSDIQMTQSPSSLSASVGDRVTITCKAS |
| | HLE molecule | | | QNVDANVAWYQQKPGQAPKSLIYSASYVYWDVPSRFSGS |
| | | | | ASGTDFTLTISSVQSEDFATYYCQQYDQQLITFGCGTKLEIKS |
| | | | | GGGGSEVQLVESGGGLVQPGGSLKLSCAASGFTFNKYAMN |
| | | | | WVRQAPGKGLEWVARIRSKYNNYATYYADSVKDRFTISRD |
| | | | | DSKNTAYLQMNNLKTEDTAVYYCVRHGNFGNSYISYWAYC |
| | | | | GQGTLVTVSSGGGGSGGGGSGGGGSQTVVTQEPSLTVSP |
| | | | | GGTVTLTCGSSTGAVTSGNYPNWVQQKPGQCPRGLIGGT |
| | | | | KFLAPGTPARFSGSLLGGKAALTLSGVQPEDEAEYYCVLWY |
| | | | | SNRWVFGGGTKLTVLGGGGDKTHTCPPCPAPELLGGPSVF |
| | | | | LFPPKPKDTLMISRTPEVTCVVVDVSHEDPEVKFNWYVDG |
| | | | | VEVHNAKTKPCEEQYGSTYRCVSVLTVLHQDWLNGKEYKC |
| | | | | KVSNKALPAPIEKTISKAKGQPREPQVYTLPPSREEMTKNQV |
| | | | | SLTCLVKGFYPSDIAVEWESNGQPENNYKTTPPVLDSDGSF |
| | | | | FLYSKLTVDKSRWQQGNVFSCSVMHEALHNHYTQKSLSLS |
| | | | | PGGGGSGGGGSGGGGSGGGGSGGGGSGGGGSDKTHTCP |
| | | | | PCPAPELLGGPSVFLFPPKPKDTLMISRTPEVTCVVVDVSHE |
| | | | | DPEVKFNWYVDGVEVHNAKTKPCEEQYGSTYRCVSVLTVL |
| | | | | HQDWLNGKEYKCKVSNKALPAPIEKTISKAKGQPREPQVYT |
| | | | | LPPSREEMTKNQVSLTCLVKGFYPSDIAVEWESNGQPENN |
| | | | | YKTTPPVLDSDGSFFLYSKLTVDKSRWQQGNVFSCSVMHE |
| | | | | ALHNHYTQKSLSLSPGK |
| 152. | PM 76-B10.11 CC | artificial | aa | DYYMY |
| | VH CDR1 | | | |
| 153. | PM 76-B10.11 CC | artificial | aa | IISDGGYYTYYSDIIKG |
| | VH CDR2 | | | |
| 154. | PM 76-B10.11 CC | artificial | aa | GFPLLRHGAMDY |
| | VH CDR3 | | | |
| 155. | PM 76-B10.11 CC | artificial | aa | KASQNVDTNVA |
| | VL CDR1 | | | |
| 156. | PM 76-B10.11 CC | artificial | aa | SASYVYW |
| | VL CDR2 | | | |
| 157. | PM 76-B10.11 CC | artificial | aa | QQYDQQLIT |
| | VL CDR3 | | | |
| 158. | PM 76-B10.11 CC | artificial | aa | QVQLVESGGGLVKPGESLRLSCAASGFTFSDYYMYWVRQA |
| | VH | | | PGKGLEWVAIISDGGYYTYYSDIIKGRFTISRDNAKNSLYLQ |
| | | | | MNSLKAEDTAVYYCARGFPLLRHGAMDYWGQGTLVTVSS |
| 159. | PM 76-B10.11 CC | artificial | aa | DIQMTQSPSSLSASVGDRVTITCKASQNVDTNVAWYQQKP |
| | VL | | | GQAPKSLIYSASYVYWDVPSRFSGSASGTDFTLTISSVQSED |
| | | | | FATYYCQQYDQQLITFGGGTKLEIK |
| 160. | PM 76-B10.11 CC | artificial | aa | QVQLVESGGGLVKPGESLRLSCAASGFTFSDYYMYWVRQA |
| | scFv | | | PGKGLEWVAIISDGGYYTYYSDIIKGRFTISRDNAKNSLYLQ |
| | | | | MNSLKAEDTAVYYCARGFPLLRHGAMDYWGQGTLVTVSS |
| | | | | GGGGSGGGGSGGGGSDIQMTQSPSSLSASVGDRVTITCKA |
| | | | | SQNVDTNVAWYQQKPGQAPKSLIYSASYVYWDVPSRFSGS |
| | | | | ASGTDFTLTISSVQSEDFATYYCQQYDQQLITFGGGTKLEIK |
| 161. | PM 76-B10.11 CC | artificial | aa | QVQLVESGGGLVKPGESLRLSCAASGFTFSDYYMYWVRQA |
| | x I2C0 bispecific | | | PGKGLEWVAIISDGGYYTYYSDIIKGRFTISRDNAKNSLYLQ |
| | molecule | | | MNSLKAEDTAVYYCARGFPLLRHGAMDYWGQGTLVTVSS |
| | | | | GGGGSGGGGSGGGGSDIQMTQSPSSLSASVGDRVTITCKA |
| | | | | SQNVDTNVAWYQQKPGQAPKSLIYSASYVYWDVPSRFSGS |
| | | | | ASGTDFTLTISSVQSEDFATYYCQQYDQQLITFGGGTKLEIKS |
| | | | | GGGGSEVQLVESGGGLVQPGGSLKLSCAASGFTFNKYAMN |
| | | | | WVRQAPGKGLEWVARIRSKYNNYATYYADSVKDRFTISRD |
| | | | | DSKNTAYLQMNNLKTEDTAVYYCVRHGNFGNSYISYWAY |
| | | | | WGQGTLVTVSSGGGGSGGGGSGGGGSQTVVTQEPSLTVS |
| | | | | PGGTVTLTCGSSTGAVTSGNYPNWVQQKPGQAPRGLIGG |
| | | | | TKFLAPGTPARFSGSLLGGKAALTLSGVQPEDEAEYYCVLW |
| | | | | YSNRWVFGGGTKLTVL |
| 162. | PM 76-B10.11 CC | artificial | aa | QVQLVESGGGLVKPGESLRLSCAASGFTFSDYYMYWVRQA |
| | x I2C0-scFc | | | PGKGLEWVAIISDGGYYTYYSDIIKGRFTISRDNAKNSLYLQ |
| | bispecific | | | MNSLKAEDTAVYYCARGFPLLRHGAMDYWGQGTLVTVSS |
| | HLE molecule | | | GGGGSGGGGSGGGGSDIQMTQSPSSLSASVGDRVTITCKA |
| | | | | SQNVDTNVAWYQQKPGQAPKSLIYSASYVYWDVPSRFSGS |
| | | | | ASGTDFTLTISSVQSEDFATYYCQQYDQQLITFGGGTKLEIKS |
| | | | | GGGGSEVQLVESGGGLVQPGGSLKLSCAASGFTFNKYAMN |
| | | | | WVRQAPGKGLEWVARIRSKYNNYATYYADSVKDRFTISRD |
| | | | | DSKNTAYLQMNNLKTEDTAVYYCVRHGNFGNSYISYWAY |
| | | | | WGQGTLVTVSSGGGGSGGGGSGGGGSQTVVTQEPSLTVS |
| | | | | PGGTVTLTCGSSTGAVTSGNYPNWVQQKPGQAPRGLIGG |
| | | | | TKFLAPGTPARFSGSLLGGKAALTLSGVQPEDEAEYYCVLW |
| | | | | YSNRWVFGGGTKLTVLGGGGDKTHTCPPCPAPELLGGPSV |
| | | | | FLFPPKPKDTLMISRTPEVTCVVVDVSHEDPEVKFNWYVDG |
| | | | | VEVHNAKTKPCEEQYGSTYRCVSVLTVLHQDWLNGKEYKC |
| | | | | KVSNKALPAPIEKTISKAKGQPREPQVYTLPPSREEMTKNQV |
| | | | | SLTCLVKGFYPSDIAVEWESNGQPENNYKTTPPVLDSDGSF |
| | | | | FLYSKLTVDKSRWQQGNVFSCSVMHEALHNHYTQKSLSLS |
| | | | | PGKGGGGSGGGGSGGGGSGGGGSGGGGSGGGGSDKTH |
| | | | | TCPPCPAPELLGGPSVFLFPPKPKDTLMISRTPEVTCVVVDV |
| | | | | SHEDPEVKFNWYVDGVEVHNAKTKPCEEQYGSTYRCVSVL |
| | | | | TVLHQDWLNGKEYKCKVSNKALPAPIEKTISKAKGQPREPQ |
| | | | | VYTLPPSREEMTKNQVSLTCLVKGFYPSDIAVEWESNGQPE |
| | | | | NNYKTTPPVLDSDGSFFLYSKLTVDKSRWQQGNVFSCSVM |
| | | | | HEALHNHYTQKSLSLSPGK |
| 163. | PM 76-B10.11 CC | artificial | aa | QVQLVESGGGLVKPGESLRLSCAASGFTFSDYYMYWVRQA |
| | × I2C0- | | | PGKGLEWVAIISDGGYYTYYSDIIKGRFTISRDNAKNSLYLQ |
| | scFc_delGK | | | MNSLKAEDTAVYYCARGFPLLRHGAMDYWGQGTLVTVSS |
| | bispecific | | | GGGGSGGGGSGGGGSDIQMTQSPSSLSASVGDRVTITCKA |
| | HLE molecule | | | SQNVDTNVAWYQQKPGQAPKSLIYSASYVYWDVPSRFSGS |
| | | | | ASGTDFTLTISSVQSEDFATYYCQQYDQQLITFGGGTKLEIKS |
| | | | | GGGGSEVQLVESGGGLVQPGGSLKLSCAASGFTFNKYAMN |
| | | | | WVRQAPGKGLEWVARIRSKYNNYATYYADSVKDRFTISRD |
| | | | | DSKNTAYLQMNNLKTEDTAVYYCVRHGNFGNSYISYWAY |
| | | | | WGQGTLVTVSSGGGGSGGGGSGGGGSQTVVTQEPSLTVS |
| | | | | PGGTVTLTCGSSTGAVTSGNYPNWVQQKPGQAPRGLIGG |
| | | | | TKFLAPGTPARFSGSLLGGKAALTLSGVQPEDEAEYYCVLW |
| | | | | YSNRWVFGGGTKLTVLGGGGDKTHTCPPCPAPELLGGPSV |
| | | | | FLFPPKPKDTLMISRTPEVTCVVVDVSHEDPEVKFNWYVDG |
| | | | | VEVHNAKTKPCEEQYGSTYRCVSVLTVLHQDWLNGKEYKC |
| | | | | KVSNKALPAPIEKTISKAKGQPREPQVYTLPPSREEMTKNQV |
| | | | | SLTCLVKGFYPSDIAVEWESNGQPENNYKTTPPVLDSDGSF |
| | | | | FLYSKLTVDKSRWQQGNVFSCSVMHEALHNHYTQKSLSLS |
| | | | | PGGGGSGGGGSGGGGSGGGGSGGGGSGGGGSDKTHTCP |
| | | | | PCPAPELLGGPSVFLFPPKPKDTLMISRTPEVTCVVVDVSHE |
| | | | | DPEVKFNWYVDGVEVHNAKTKPCEEQYGSTYRCVSVLTVL |
| | | | | HQDWLNGKEYKCKVSNKALPAPIEKTISKAKGQPREPQVYT |
| | | | | LPPSREEMTKNQVSLTCLVKGFYPSDIAVEWESNGQPENN |
| | | | | YKTTPPVLDSDGSFFLYSKLTVDKSRWQQGNVFSCSVMHE |
| | | | | ALHNHYTQKSLSLSPGK |
| 164. | PM 76-B10.11 CC | artificial | aa | QVQLVESGGGLVKPGESLRLSCAASGFTFSDYYMYWVRQA |
| | x 12C0 CC | | | PGKGLEWVAIISDGGYYTYYSDIIKGRFTISRDNAKNSLYLQ |
| | (103/43)-scFc | | | MNSLKAEDTAVYYCARGFPLLRHGAMDYWGQGTLVTVSS |
| | bispecific | | | GGGGSGGGGSGGGGSDIQMTQSPSSLSASVGDRVTITCKA |
| | molecule | | | SQNVDTNVAWYQQKPGQAPKSLIYSASYVYWDVPSRFSGS |
| | | | | ASGTDFTLTISSVQSEDFATYYCQQYDQQLITFGGGTKLEIKS |
| | | | | GGGGSEVQLVESGGGLVQPGGSLKLSCAASGFTFNKYAMN |
| | | | | WVRQAPGKGLEWVARIRSKYNNYATYYADSVKDRFTISRD |
| | | | | DSKNTAYLQMNNLKTEDTAVYYCVRHGNFGNSYISYWAYC |
| | | | | GQGTLVTVSSGGGGSGGGGSGGGGSQTVVTQEPSLTVSP |
| | | | | GGTVTLTCGSSTGAVTSGNYPNWVQQKPGQCPRGLIGGT |
| | | | | KFLAPGTPARFSGSLLGGKAALTLSGVQPEDEAEYYCVLWY |
| | | | | SNRWVFGGGTKLTVL |
| 165. | PM 76-B10.11 CC | artificial | aa | QVQLVESGGGLVKPGESLRLSCAASGFTFSDYYMYWVRQA |
| | x I2C0 CC | | | PGKGLEWVAIISDGGYYTYYSDIIKGRFTISRDNAKNSLYLQ |
| | (103/43)-scFc | | | MNSLKAEDTAVYYCARGFPLLRHGAMDYWGQGTLVTVSS |
| | bispecific | | | GGGGSGGGGSGGGGSDIQMTQSPSSLSASVGDRVTITCKA |
| | HLE molecule | | | SQNVDTNVAWYQQKPGQAPKSLIYSASYVYWDVPSRFSGS |
| | | | | ASGTDFTLTISSVQSEDFATYYCQQYDQQLITFGGGTKLEIKS |
| | | | | GGGGSEVQLVESGGGLVQPGGSLKLSCAASGFTFNKYAMN |
| | | | | WVRQAPGKGLEWVARIRSKYNNYATYYADSVKDRFTISRD |
| | | | | DSKNTAYLQMNNLKTEDTAVYYCVRHGNFGNSYISYWAYC |
| | | | | GQGTLVTVSSGGGGSGGGGSGGGGSQTVVTQEPSLTVSP |
| | | | | GGTVTLTCGSSTGAVTSGNYPNWVQQKPGQCPRGLIGGT |
| | | | | KFLAPGTPARFSGSLLGGKAALTLSGVQPEDEAEYYCVLWY |
| | | | | SNRWVFGGGTKLTVLGGGGDKTHTCPPCPAPELLGGPSVF |
| | | | | LFPPKPKDTLMISRTPEVTCVVVDVSHEDPEVKFNWYVDG |
| | | | | VEVHNAKTKPCEEQYGSTYRCVSVLTVLHQDWLNGKEYKC |
| | | | | KVSNKALPAPIEKTISKAKGQPREPQVYTLPPSREEMTKNQV |
| | | | | SLTCLVKGFYPSDIAVEWESNGQPENNYKTTPPVLDSDGSF |
| | | | | FLYSKLTVDKSRWQQGNVFSCSVMHEALHNHYTQKSLSLS |
| | | | | PGKGGGGSGGGGSGGGGSGGGGSGGGGSGGGGSDKTH |
| | | | | TCPPCPAPELLGGPSVFLFPPKPKDTLMISRTPEVTCVVVDV |
| | | | | SHEDPEVKFNWYVDGVEVHNAKTKPCEEQYGSTYRCVSVL |
| | | | | TVLHQDWLNGKEYKCKVSNKALPAPIEKTISKAKGQPREPQ |
| | | | | VYTLPPSREEMTKNQVSLTCLVKGFYPSDIAVEWESNGQPE |
| | | | | NNYKTTPPVLDSDGSFFLYSKLTVDKSRWQQGNVFSCSVM |
| | | | | HEALHNHYTQKSLSLSPGK |
| 166. | PM 76-B10.11 CC | artificial | aa | QVQLVESGGGLVKPGESLRLSCAASGFTFSDYYMYWVRQA |
| | x I2C0 CC | | | PGKGLEWVAIISDGGYYTYYSDIIKGRFTISRDNAKNSLYLQ |
| | (103/43)- | | | MNSLKAEDTAVYYCARGFPLLRHGAMDYWGQGTLVTVSS |
| | scFc_delGK | | | GGGGSGGGGSGGGGSDIQMTQSPSSLSASVGDRVTITCKA |
| | bispecific | | | SQNVDTNVAWYQQKPGQAPKSLIYSASYVYWDVPSRFSGS |
| | HLE molecule | | | ASGTDFTLTISSVQSEDFATYYCQQYDQQLITFGGGTKLEIKS |
| | | | | GGGGSEVQLVESGGGLVQPGGSLKLSCAASGFTFNKYAMN |
| | | | | WVRQAPGKGLEWVARIRSKYNNYATYYADSVKDRFTISRD |
| | | | | DSKNTAYLQMNNLKTEDTAVYYCVRHGNFGNSYISYWAYC |
| | | | | GQGTLVTVSSGGGGSGGGGSGGGGSQTVVTQEPSLTVSP |
| | | | | GGTVTLTCGSSTGAVTSGNYPNWVQQKPGQCPRGLIGGT |
| | | | | KFLAPGTPARFSGSLLGGKAALTLSGVQPEDEAEYYCVLWY |
| | | | | SNRWVFGGGTKLTVLGGGGDKTHTCPPCPAPELLGGPSVF |
| | | | | LFPPKPKDTLMISRTPEVTCVVVDVSHEDPEVKFNWYVDG |
| | | | | VEVHNAKTKPCEEQYGSTYRCVSVLTVLHQDWLNGKEYKC |
| | | | | KVSNKALPAPIEKTISKAKGQPREPQVYTLPPSREEMTKNQV |
| | | | | SLTCLVKGFYPSDIAVEWESNGQPENNYKTTPPVLDSDGSF |
| | | | | FLYSKLTVDKSRWQQGNVFSCSVMHEALHNHYTQKSLSLS |
| | | | | PGGGGSGGGGSGGGGSGGGGSGGGGSGGGGSDKTHTCP |
| | | | | PCPAPELLGGPSVFLFPPKPKDTLMISRTPEVTCVVVDVSHE |
| | | | | DPEVKFNWYVDGVEVHNAKTKPCEEQYGSTYRCVSVLTVL |
| | | | | HQDWLNGKEYKCKVSNKALPAPIEKTISKAKGQPREPQVYT |
| | | | | LPPSREEMTKNQVSLTCLVKGFYPSDIAVEWESNGQPENN |
| | | | | YKTTPPVLDSDGSFFLYSKLTVDKSRWQQGNVFSCSVMHE |
| | | | | ALHNHYTQKSLSLSPGK |
| 167. | PM 76-B10.11 CC | artificial | aa | DYYMY |
| | x I2C0-scFc VH | | | |
| | CDR1 | | | |
| 168. | PM 76-B10.11 CC | artificial | aa | IISDGGYYTYYSDIIKG |
| | x I2C0-scFc VH | | | |
| | CDR2 | | | |
| 169. | PM 76-B10.11 CC | artificial | aa | GFPLLRHGAMDY |
| | x 12C0-scFc VH | | | |
| | CDR3 | | | |
| 170. | PM 76-B10.11 CC | artificial | aa | KASQNVDTNVA |
| | x I2C0-scFc VL | | | |
| | CDR1 | | | |
| 171. | PM 76-B10.11 CC | artificial | aa | SASYVYW |
| | x I2C0-scFc VL | | | |
| | CDR2 | | | |
| 172. | PM 76-B10.11 CC | artificial | aa | QQYDQQLIT |
| | x I2C0-scFc VL | | | |
| | CDR3 | | | |
| 173. | PM 76-B10.11 CC | artificial | aa | QVQLVESGGGLVKPGESLRLSCAASGFTFSDYYMYWVRQA |
| | x I2C0-scFc VH | | | PGKCLEWVAIISDGGYYTYYSDIIKGRFTISRDNAKNSLYLQ |
| | | | | MNSLKAEDTAVYYCARGFPLLRHGAMDYWGQGTLVTVSS |
| 174. | PM 76-B10.11 CC | artificial | aa | DIQMTQSPSSLSASVGDRVTITCKASQNVDTNVAWYQQKP |
| | x I2C0-scFc VL | | | GQAPKSLIYSASYVYWDVPSRFSGSASGTDFTLTISSVQSED |
| | | | | FATYYCQQYDQQLITFGCGTKLEIK |
| 175. | PM 76-B10.11 CC | artificial | aa | QVQLVESGGGLVKPGESLRLSCAASGFTFSDYYMYWVRQA |
| | x I2C0-scFc scFv | | | PGKCLEWVAIISDGGYYTYYSDIIKGRFTISRDNAKNSLYLQ |
| | | | | MNSLKAEDTAVYYCARGFPLLRHGAMDYWGQGTLVTVSS |
| | | | | GGGGSGGGGSGGGGSDIQMTQSPSSLSASVGDRVTITCKA |
| | | | | SQNVDTNVAWYQQKPGQAPKSLIYSASYVYWDVPSRFSGS |
| | | | | ASGTDFTLTISSVQSEDFATYYCQQYDQQLITFGCGTKLEIK |
| 176. | PM 76-B10.11 CC | artificial | aa | QVQLVESGGGLVKPGESLRLSCAASGFTFSDYYMYWVRQA |
| | x I2C0-scFc | | | PGKCLEWVAIISDGGYYTYYSDIIKGRFTISRDNAKNSLYLQ |
| | bispecific | | | MNSLKAEDTAVYYCARGFPLLRHGAMDYWGQGTLVTVSS |
| | molecule | | | GGGGSGGGGSGGGGSDIQMTQSPSSLSASVGDRVTITCKA |
| | | | | SQNVDTNVAWYQQKPGQAPKSLIYSASYVYWDVPSRFSGS |
| | | | | ASGTDFTLTISSVQSEDFATYYCQQYDQQLITFGCGTKLEIKS |
| | | | | GGGGSEVQLVESGGGLVQPGGSLKLSCAASGFTFNKYAMN |
| | | | | WVRQAPGKGLEWVARIRSKYNNYATYYADSVKDRFTISRD |
| | | | | DSKNTAYLQMNNLKTEDTAVYYCVRHGNFGNSYISYWAY |
| | | | | WGQGTLVTVSSGGGGSGGGGSGGGGSQTVVTQEPSLTVS |
| | | | | PGGTVTLTCGSSTGAVTSGNYPNWVQQKPGQAPRGLIGG |
| | | | | TKFLAPGTPARFSGSLLGGKAALTLSGVQPEDEAEYYCVLW |
| | | | | YSNRWVFGGGTKLTVL |
| 177. | PM 76-B10.11 CC | artificial | aa | QVQLVESGGGLVKPGESLRLSCAASGFTFSDYYMYWVRQA |
| | x I2C0-scFc | | | PGKCLEWVAIISDGGYYTYYSDIIKGRFTISRDNAKNSLYLQ |
| | bispecific | | | MNSLKAEDTAVYYCARGFPLLRHGAMDYWGQGTLVTVSS |
| | HLE molecule | | | GGGGSGGGGSGGGGSDIQMTQSPSSLSASVGDRVTITCKA |
| | | | | SQNVDTNVAWYQQKPGQAPKSLIYSASYVYWDVPSRFSGS |
| | | | | ASGTDFTLTISSVQSEDFATYYCQQYDQQLITFGCGTKLEIKS |
| | | | | GGGGSEVQLVESGGGLVQPGGSLKLSCAASGFTFNKYAMN |
| | | | | WVRQAPGKGLEWVARIRSKYNNYATYYADSVKDRFTISRD |
| | | | | DSKNTAYLQMNNLKTEDTAVYYCVRHGNFGNSYISYWAY |
| | | | | WGQGTLVTVSSGGGGSGGGGSGGGGSQTVVTQEPSLTVS |
| | | | | PGGTVTLTCGSSTGAVTSGNYPNWVQQKPGQAPRGLIGG |
| | | | | TKFLAPGTPARFSGSLLGGKAALTLSGVQPEDEAEYYCVLW |
| | | | | YSNRWVFGGGTKLTVLGGGGDKTHTCPPCPAPELLGGPSV |
| | | | | FLFPPKPKDTLMISRTPEVTCVVVDVSHEDPEVKFNWYVDG |
| | | | | VEVHNAKTKPCEEQYGSTYRCVSVLTVLHQDWLNGKEYKC |
| | | | | KVSNKALPAPIEKTISKAKGQPREPQVYTLPPSREEMTKNQV |
| | | | | SLTCLVKGFYPSDIAVEWESNGQPENNYKTTPPVLDSDGSF |
| | | | | FLYSKLTVDKSRWQQGNVFSCSVMHEALHNHYTQKSLSLS |
| | | | | PGKGGGGSGGGGSGGGGGGGGSGGGGSGGGGSDKTH |
| | | | | TCPPCPAPELLGGPSVFLFPPKPKDTLMISRTPEVTCVVVDV |
| | | | | SHEDPEVKFNWYVDGVEVHNAKTKPCEEQYGSTYRCVSVL |
| | | | | TVLHQDWLNGKEYKCKVSNKALPAPIEKTISKAKGQPREPQ |
| | | | | VYTLPPSREEMTKNQVSLTCLVKGFYPSDIAVEWESNGQPE |
| | | | | NNYKTTPPVLDSDGSFFLYSKLTVDKSRWQQGNVFSCSVM |
| | | | | HEALHNHYTQKSLSLSPGK |
| 178. | PM 76-B10.11 CC | artificial | aa | QVQLVESGGGLVKPGESLRLSCAASGFTFSDYYMYWVRQA |
| | × I2C0- | | | PGKCLEWVAIISDGGYYTYYSDIIKGRFTISRDNAKNSLYLQ |
| | scFc_delGK | | | MNSLKAEDTAVYYCARGFPLLRHGAMDYWGQGTLVTVSS |
| | bispecific | | | GGGGSGGGGSGGGGSDIQMTQSPSSLSASVGDRVTITCKA |
| | HLE molecule | | | SQNVDTNVAWYQQKPGQAPKSLIYSASYVYWDVPSRFSGS |
| | | | | ASGTDFTLTISSVQSEDFATYYCQQYDQQLITFGCGTKLEIKS |
| | | | | GGGGSEVQLVESGGGLVQPGGSLKLSCAASGFTFNKYAMN |
| | | | | WVRQAPGKGLEWVARIRSKYNNYATYYADSVKDRFTISRD |
| | | | | DSKNTAYLQMNNLKTEDTAVYYCVRHGNFGNSYISYWAY |
| | | | | WGQGTLVTVSSGGGGSGGGGSGGGGSQTVVTQEPSLTVS |
| | | | | PGGTVTLTCGSSTGAVTSGNYPNWVQQKPGQAPRGLIGG |
| | | | | TKFLAPGTPARFSGSLLGGKAALTLSGVQPEDEAEYYCVLW |
| | | | | YSNRWVFGGGTKLTVLGGGGDKTHTCPPCPAPELLGGPSV |
| | | | | FLFPPKPKDTLMISRTPEVTCVVVDVSHEDPEVKFNWYVDG |
| | | | | VEVHNAKTKPCEEQYGSTYRCVSVLTVLHQDWLNGKEYKC |
| | | | | KVSNKALPAPIEKTISKAKGQPREPQVYTLPPSREEMTKNQV |
| | | | | SLTCLVKGFYPSDIAVEWESNGQPENNYKTTPPVLDSDGSF |
| | | | | FLYSKLTVDKSRWQQGNVFSCSVMHEALHNHYTQKSLSLS |
| | | | | PGGGGSGGGGSGGGGSGGGGSGGGGSGGGGSDKTHTCP |
| | | | | PCPAPELLGGPSVFLFPPKPKDTLMISRTPEVTCVVVDVSHE |
| | | | | DPEVKFNWYVDGVEVHNAKTKPCEEQYGSTYRCVSVLTVL |
| | | | | HQDWLNGKEYKCKVSNKALPAPIEKTISKAKGQPREPQVYT |
| | | | | LPPSREEMTKNQVSLTCLVKGFYPSDIAVEWESNGQPENN |
| | | | | YKTTPPVLDSDGSFFLYSKLTVDKSRWQQGNVFSCSVMHE |
| | | | | ALHNHYTQKSLSLSPGK |
| 179. | PM 76-B10.11 CC | artificial | aa | QVQLVESGGGLVKPGESLRLSCAASGFTFSDYYMYWVRQA |
| | x I2C0 CC | | | PGKCLEWVAIISDGGYYTYYSDIIKGRFTISRDNAKNSLYLQ |
| | (103/43)-scFc | | | MNSLKAEDTAVYYCARGFPLLRHGAMDYWGQGTLVTVSS |
| | bispecific | | | GGGGSGGGGSGGGGSDIQMTQSPSSLSASVGDRVTITCKA |
| | molecule | | | SQNVDTNVAWYQQKPGQAPKSLIYSASYVYWDVPSRFSGS |
| | | | | ASGTDFTLTISSVQSEDFATYYCQQYDQQLITFGCGTKLEIKS |
| | | | | GGGGSEVQLVESGGGLVQPGGSLKLSCAASGFTFNKYAMN |
| | | | | WVRQAPGKGLEWVARIRSKYNNYATYYADSVKDRFTISRD |
| | | | | DSKNTAYLQMNNLKTEDTAVYYCVRHGNFGNSYISYWAYC |
| | | | | GQGTLVTVSSGGGGSGGGGSGGGGSQTVVTQEPSLTVSP |
| | | | | GGTVTLTCGSSTGAVTSGNYPNWVQQKPGQCPRGLIGGT |
| | | | | KFLAPGTPARFSGSLLGGKAALTLSGVQPEDEAEYYCVLWY |
| | | | | SNRWVFGGGTKLTVL |
| 180. | PM 76-B10.11 CC | artificial | aa | QVQLVESGGGLVKPGESLRLSCAASGFTFSDYYMYWVRQA |
| | x 12C0 CC | | | PGKCLEWVAIISDGGYYTYYSDIIKGRFTISRDNAKNSLYLQ |
| | (103/43)-scFc | | | MNSLKAEDTAVYYCARGFPLLRHGAMDYWGQGTLVTVSS |
| | bispecific | | | GGGGSGGGGSGGGGSDIQMTQSPSSLSASVGDRVTITCKA |
| | HLE molecule | | | SQNVDTNVAWYQQKPGQAPKSLIYSASYVYWDVPSRFSGS |
| | | | | ASGTDFTLTISSVQSEDFATYYCQQYDQQLITFGCGTKLEIKS |
| | | | | GGGGSEVQLVESGGGLVQPGGSLKLSCAASGFTFNKYAMN |
| | | | | WVRQAPGKGLEWVARIRSKYNNYATYYADSVKDRFTISRD |
| | | | | DSKNTAYLQMNNLKTEDTAVYYCVRHGNFGNSYISYWAYC |
| | | | | GQGTLVTVSSGGGGSGGGGSGGGGSQTVVTQEPSLTVSP |
| | | | | GGTVTLTCGSSTGAVTSGNYPNWVQQKPGQCPRGLIGGT |
| | | | | KFLAPGTPARFSGSLLGGKAALTLSGVQPEDEAEYYCVLWY |
| | | | | SNRWVFGGGTKLTVLGGGGDKTHTCPPCPAPELLGGPSVF |
| | | | | LFPPKPKDTLMISRTPEVTCVVVDVSHEDPEVKFNWYVDG |
| | | | | VEVHNAKTKPCEEQYGSTYRCVSVLTVLHQDWLNGKEYKC |
| | | | | KVSNKALPAPIEKTISKAKGQPREPQVYTLPPSREEMTKNQV |
| | | | | SLTCLVKGFYPSDIAVEWESNGQPENNYKTTPPVLDSDGSF |
| | | | | FLYSKLTVDKSRWQQGNVFSCSVMHEALHNHYTQKSLSLS |
| | | | | PGKGGGGSGGGGSGGGGSGGGGSGGGGSGGGGSDKTH |
| | | | | TCPPCPAPELLGGPSVFLFPPKPKDTLMISRTPEVTCVVVDV |
| | | | | SHEDPEVKFNWYVDGVEVHNAKTKPCEEQYGSTYRCVSVL |
| | | | | TVLHQDWLNGKEYKCKVSNKALPAPIEKTISKAKGQPREPQ |
| | | | | VYTLPPSREEMTKNQVSLTCLVKGFYPSDIAVEWESNGQPE |
| | | | | NNYKTTPPVLDSDGSFFLYSKLTVDKSRWQQGNVFSCSVM |
| | | | | HEALHNHYTQKSLSLSPGK |
| 181. | PM 76-B10.11 CC | artificial | aa | QVQLVESGGGLVKPGESLRLSCAASGFTFSDYYMYWVRQA |
| | x 12C0 CC | | | PGKCLEWVAIISDGGYYTYYSDIIKGRFTISRDNAKNSLYLQ |
| | (103/43)- | | | MNSLKAEDTAVYYCARGFPLLRHGAMDYWGQGTLVTVSS |
| | scFc_delGK | | | GGGGSGGGGSGGGGSDIQMTQSPSSLSASVGDRVTITCKA |
| | bispecific | | | SQNVDTNVAWYQQKPGQAPKSLIYSASYVYWDVPSRFSGS |
| | HLE molecule | | | ASGTDFTLTISSVQSEDFATYYCQQYDQQLITFGCGTKLEIKS |
| | | | | GGGGSEVQLVESGGGLVQPGGSLKLSCAASGFTFNKYAMN |
| | | | | WVRQAPGKGLEWVARIRSKYNNYATYYADSVKDRFTISRD |
| | | | | DSKNTAYLQMNNLKTEDTAVYYCVRHGNFGNSYISYWAYC |
| | | | | GQGTLVTVSSGGGGSGGGGSGGGGSQTVVTQEPSLTVSP |
| | | | | GGTVTLTCGSSTGAVTSGNYPNWVQQKPGQCPRGLIGGT |
| | | | | KFLAPGTPARFSGSLLGGKAALTLSGVQPEDEAEYYCVLWY |
| | | | | SNRWVFGGGTKLTVLGGGGDKTHTCPPCPAPELLGGPSVF |
| | | | | LFPPKPKDTLMISRTPEVTCVVVDVSHEDPEVKFNWYVDG |
| | | | | VEVHNAKTKPCEEQYGSTYRCVSVLTVLHQDWLNGKEYKC |
| | | | | KVSNKALPAPIEKTISKAKGQPREPQVYTLPPSREEMTKNQV |
| | | | | SLTCLVKGFYPSDIAVEWESNGQPENNYKTTPPVLDSDGSF |
| | | | | FLYSKLTVDKSRWQQGNVFSCSVMHEALHNHYTQKSLSLS |
| | | | | PGGGGSGGGGSGGGGSGGGGSGGGGSGGGGSDKTHTCP |
| | | | | PCPAPELLGGPSVFLFPPKPKDTLMISRTPEVTCVVVDVSHE |
| | | | | DPEVKFNWYVDGVEVHNAKTKPCEEQYGSTYRCVSVLTVL |
| | | | | HQDWLNGKEYKCKVSNKALPAPIEKTISKAKGQPREPQVYT |
| | | | | LPPSREEMTKNQVSLTCLVKGFYPSDIAVEWESNGQPENN |
| | | | | YKTTPPVLDSDGSFFLYSKLTVDKSRWQQGNVFSCSVMHE |
| | | | | ALHNHYTQKSLSLSPGK |
| 182. | CD70_21D_CC | artificial | aa | TYAMS |
| | VH CDR1 | | | |
| 183. | CD70_21D_CC | artificial | aa | AISGSGGRTFYAESVEG |
| | VH CDR2 | | | |
| 184. | CD70_21D_CC | artificial | aa | HDYSNYPYFDY |
| | VH CDR3 | | | |
| 185. | CD70_21D_CC VL | artificial | aa | RASQSVRSTYLA |
| | CDR1 | | | |
| 186. | CD70_21D_CC VL | artificial | aa | GASSRAT |
| | CDR2 | | | |
| 187. | CD70_21D_CC VL | artificial | aa | QQYGDLPFT |
| | CDR3 | | | |
| 188. | CD70_21D_CC | artificial | aa | EVQLLESGGGMVQPGGSLRLSCAASGFTFSTYAMSWVRQ |
| | VH | | | APGKCLEWVSAISGSGGRTFYAESVEGRFTISRDNSKNTLYL |
| | | | | QMNSLRAEDTAVYYCAKHDYSNYPYFDYWGQGTLVTVSS |
| 189. | CD70_21D_CC VL | artificial | aa | EIVLTQSPGTLSLSPGERATLSCRASQSVRSTYLAWYQQK |
| | | | | PGQAPRLLIYGASSRATGIPDRFSGSGSGTDFTLTISRLE |
| | | | | PEDFAVYSCQQYGDLPFTFGCGTKLEIK |
| 190. | CD70_21D_CCx1 | artificial | aa | EVQLLESGGGMVQPGGSLRLSCAASGFTFSTYAMSWVRQ |
| | 2C scFc | | | APGKCLEWVSAISGSGGRTFYAESVEGRFTISRDNSKNTLYL |
| | | | | QMNSLRAEDTAVYYCAKHDYSNYPYFDYWGQGTLVTVSS |
| | | | | GGGGSGGGGSGGGGSEIVLTQSPGTLSLSPGERATLSCRAS |
| | | | | QSVRSTYLAWYQQKPGQAPRLLIYGASSRATGIPDRFSGSG |
| | | | | SGTDFTLTISRLEPEDFAVYSCQQYGDLPFTFGCGTKLEIKSG |
| | | | | GGGSEVQLVESGGGLVQPGGSLKLSCAASGFTFNKYAMN |
| | | | | WVRQAPGKGLEWVARIRSKYNNYATYYADSVKDRFTISRD |
| | | | | DSKNTAYLQMNNLKTEDTAVYYCVRHGNFGNSYISYWAY |
| | | | | WGQGTLVTVSSGGGGSGGGGSGGGGSQTVVTQEPSLTVS |
| | | | | PGGTVTLTCGSSTGAVTSGNYPNWVQQKPGQAPRGLIGG |
| | | | | TKFLAPGTPARFSGSLLGGKAALTLSGVQPEDEAEYYCVLW |
| | | | | YSNRWVFGGGTKLTVLGGGGDKTHTCPPCPAPELLGGPSV |
| | | | | FLFPPKPKDTLMISRTPEVTCVVVDVSHEDPEVKFNWYVDG |
| | | | | VEVHNAKTKPCEEQYGSTYRCVSVLTVLHQDWLNGKEYKC |
| | | | | KVSNKALPAPIEKTISKAKGQPREPQVYTLPPSREEMTKNQV |
| | | | | SLTCLVKGFYPSDIAVEWESNGQPENNYKTTPPVLDSDGSF |
| | | | | FLYSKLTVDKSRWQQGNVFSCSVMHEALHNHYTQKSLSLS |
| | | | | PGKGGGGSGGGGSGGGGSGGGGSGGGGSGGGGSDKTH |
| | | | | TCPPCPAPELLGGPSVFLFPPKPKDTLMISRTPEVTCVVVDV |
| | | | | SHEDPEVKFNWYVDGVEVHNAKTKPCEEQYGSTYRCVSVL |
| | | | | TVLHQDWLNGKEYKCKVSNKALPAPIEKTISKAKGQPREPQ |
| | | | | VYTLPPSREEMTKNQVSLTCLVKGFYPSDIAVEWESNGQPE |
| | | | | NNYKTTPPVLDSDGSFFLYSKLTVDKSRWQQGNVFSCSVM |
| | | | | HEALHNHYTQKSLSLSPGK |
| 191. | hinge sequence | artificial | aa | DKTHTCPPCP |
| 192 | hinge sequence | artificial | aa | ERKCCVECPPCP |
| 193 | hinge sequence | artificial | aa | ELKTPLDTTHTCPRCP |
| 194 | hinge sequence | artificial | aa | ELKTPLGDTTHTCPRCP |
| 195 | hinge sequence | artificial | aa | ESKYGPPCPSCP |
| 196 | hinge sequence | artificial | aa | EPKSCDKTHTCPPCP |
| 197 | linker | artificial | aa | GGGGS |
| 198 | His-tag | artificial | aa | HHHHHH |
| 199 | linker | artificial | aa | GGGGSGGGGSGGGGS |

The invention will be more fully understood by reference to the following examples. The examples should not, however, be construed as limiting the scope of the invention.

### EXAMPLES

### Example 1: Increased Aggregation of BiTE Molecules when Stored Under Frozen Conditions (−20° C.

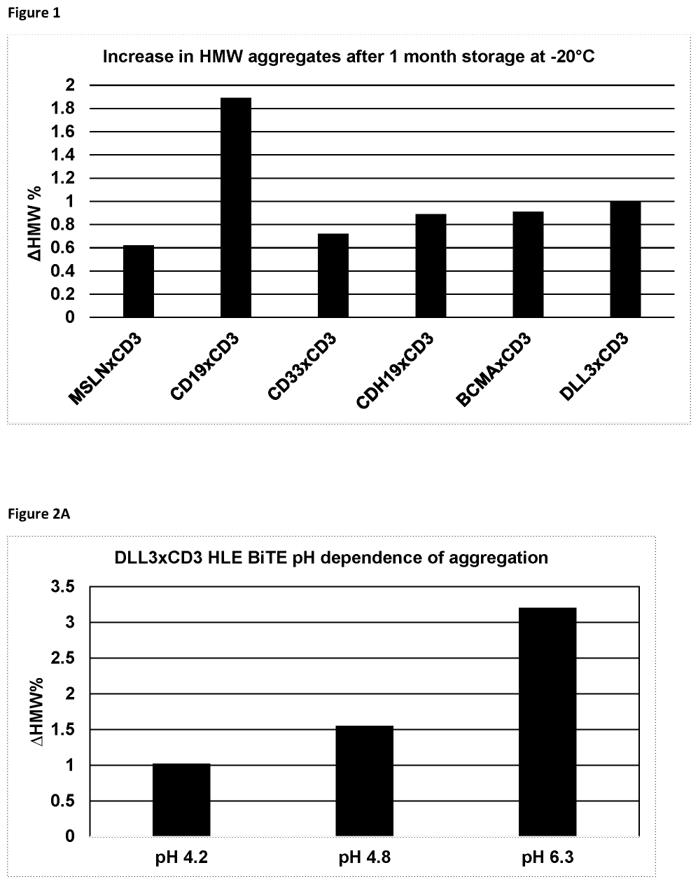

Compositions comprising 1 mg/mL each of the HLE BITEs (MSLN×CD3, CD19×CD3, CD33×CD3, BCMA×CD3, and DLL3×CD3) and having a pH of pH 4.2 were filled in 5 mL vials and stored at −20° C. for one month. After one month, the compositions were thawed at room temperature and analyzed immediately by SE-UHPLC to determine the levels of HMW aggregates (e.g., bispecific antibody dimers) in the compositions. SE-UHPLC separates proteins in solution based on their hydrodynamic volume using a size exclusion ultra high performance analytical column. The high molecular weight (aggregate peaks) elutes earlier than the monomeric and lower molecular weight peaks. Components are eluted isocratically, detected by UV detection, integrated, and the results are reported as relative peak area percentages of high molecular weight, main peak, and low molecular weight peaks. As shown in FIG. 1, the HMW aggregate levels increased in each composition after the storage.

### Example 2: pH and Temperature Dependence of Aggregation of BiTE Molecules

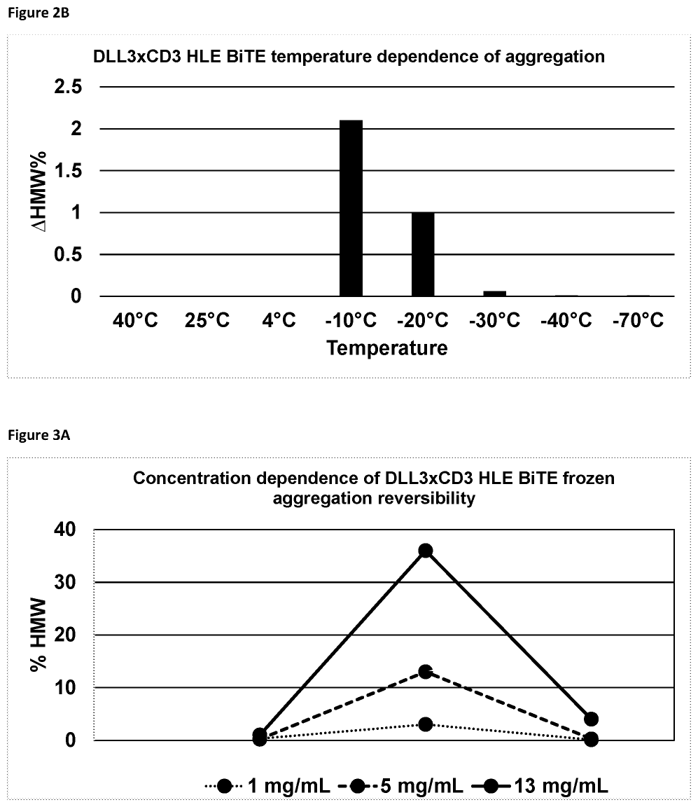

Compositions comprising the DLL3×CD3 HLE BiTE at a concentration of 1 mg/mL and having a pH of 4.2, 4.8 or 6.3 were filled in 5 mL vials and stored at −20° C. for one month. After one month, the compositions were thawed at room temperature and analyzed immediately by SE-UHPLC to determine the levels of HMW aggregates and the results are shown in FIG. 2A. HMW aggregates formed during the storage appears to be pH dependent (FIG. 2A).

Also, compositions comprising the DLL3×CD3 HLE BiTE at a concentration of 1 mg/mL were filled in 5 mL vials and stored at −10° C., −20° C., −30° C., −40° C., and −70° C. for one month. After one month, the compositions were thawed at room temperature and analyzed immediately by SE-UHPLC to determine the levels of HMW aggregates and the results are shown in FIG. 2B. HMW aggregate levels increased when the BiTE was stored at −10° C., −20° C., −30° C., little increase in HMW aggregate levels was observed when the BiTE was stored at a temperature lower than the Tg′ (−32° C.) of the composition comprising the BiTE (FIG. 2B).

### Example 3: Holding Thawed BiTEs at a Temperature of Between 15° C. and 30° C. Reduces Aggregation Levels

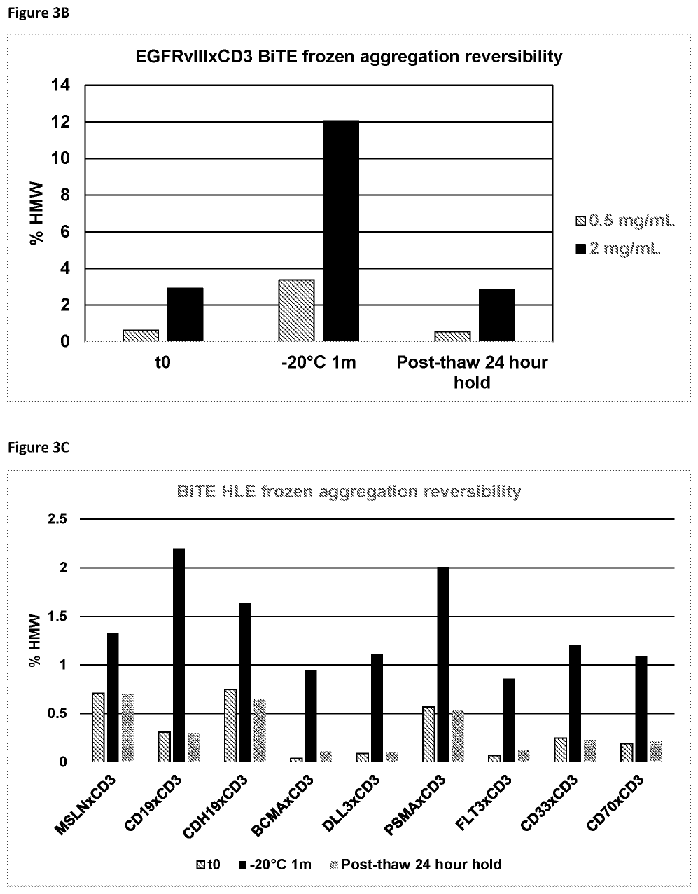

Compositions comprising the DLL3×CD3 HLE BiTE at a concentration of 1 mg/mL, 5 mg/mL and 13 mg/mL were filled in 5 mL vials and stored at −20° C. for 12 months. After 12 months, the compositions were thawed at room temperature and analyzed immediately by SE-UHPLC to determine the levels of HMW aggregates. The thawed compositions were held at room temperature for an additional 24 hours and the HMW aggregate levels were analyzed again using SE-UHPLC. The HMW aggregate levels decreased to below 5% after the holding period (FIG. 3A).

In a second series of experiments, compositions comprising the EGFRvIII×CD3 BiTE at a concentration of 0.5 mg/mL and 2 mg/mL were filled in 5 mL vials and stored at −20° C. for one month. After month, the compositions were thawed at room temperature and analyzed immediately by SE-UHPLC to determine the levels of HMW aggregates. The thawed compositions were held at room temperature for an additional 24 hours and the HMW aggregate levels were analyzed again using SE-UHPLC. The HMW aggregate levels decreased to below 5% after the holding period (FIG. 3B).

In a third series of experiments, compositions comprising each of the HLE BITEs (MSLN×CD3, CD19×CD3, CD33×CD3, CDH19×CD3, BCMA×CD3, DLL3×CD3, FLT3×CD3, PSMA×CD3, and CD70×CD3) at a concentration of 1 mg/mL were filled in 5 mL vials and stored at −20° C. for one month. After one month, the compositions were thawed at room temperature and analyzed immediately by SE-UHPLC to determine the levels of HMW aggregates. The thawed compositions were held at room temperature for an additional 24 hours and the HMW aggregate levels were analyzed again using SE-UHPLC. The HMW aggregate levels in each composition decreased to below 1% after the holding period (FIG. 3C).

In a forth series of experiments, compositions comprising each of the HLE BITEs (CD33×CD3 and DLL3×CD3) at a concentration of 1 mg/mL were filled in 5 mL and stored at −30° C. for 18 months. After 18 months, the compositions were thawed at room temperature and analyzed immediately by SE-UHPLC to determine the levels of HMW aggregates. The thawed compositions were held at room temperature for an additional 24 hours and the HMW aggregate levels were analyzed again using SE-UHPLC. The HMW aggregate levels in each composition decreased to below 0.4% after the holding period (FIG. 3D).

### Example 4: Holding Thawed BiTEs at a Temperature of Between 15° C. and 30° C. has No Impact on Stability Attributes of the BiTEs

Compositions comprising the DLL3×CD3 HLE BiTE at a concentration of 13 mg/mL were filled in 5 mL vials and stored at −20° C. for 12 months. After 12 months, the compositions were thawed at room temperature, and the thawed compositions were held at room temperature for an additional 24 hours. Several stability attributes were analyzed after the 24-hour holding period and the results are shown in Table 4.

The potency of the HLE BiTE molecules was measured by a cell-based bioassay that measures cell death by the loss of luminescence in a carcinoma cell line. The biological activity of the test sample is determined by comparing the test sample response to that of the reference standard (relative potency).

Clipping of the BiTE molecules was measured using Reduced Capillary Electrophoresis-Sodium Dodecyl Sulfate (rCE-SDS). rCE-SDS separates proteins based on their hydrodynamic sizes under reducing and denaturing conditions. Proteins are denatured, reduced, and injected into bare fused silica capillary filled with a polymer gel matrix. An electrical voltage is applied across the capillary and the SDS-coated proteins are separated based on their hydrodynamic size; the smaller size proteins migrating earlier than the larger sized proteins. Proteins are detected using a photodiode array (PDA) detector, integrated, and the results are reported as relative peak area percentages of low molecular weight, main peak, and high molecular weight peaks.

Charge variants of the BiTE molecules were measured using Cation Exchange High Performance Liquid Chromatography (CEX-HPLC). Charge variants of the protein were eluted using a mobile phase gradient of increasing ionic strength, under appropriate pH. Proteins with less positive surface charge elute earlier than proteins with a greater positive surface charge. Eluted charge variants were detected by UV detection, integrated, and the results for Main, Acidic, and Basic peaks were reported as the percentage of the total peak area.

As shown in Table 4, holding the thawed BiTE had no negative impact on the stability attributes while the levels of HMW decreased after the holding period.

TABLE 4
DLL3 × CD3 HLE BiTE (13 mg/mL)

| | Attributes | | | |
|---|---|---|---|---|
| | Aggregation | Potency | Clipping | Charge variants |
| Time | (SE-HPLC) | (Bioassay) | (rCE-SDS) | (CEX-HPLC) |
| T₀ | 1.0% HMW | 102% RP | 1.4% LMW | 80.8% MP |
| −20° C., | 36.0% HMW | n/a | n/a | n/a |
| 12 months | | | | |
| 24 hr. | 4.0% HMW | 95% RP | 1.2% LMW | 80.5% MP |
| post-thaw | | | | |
| RT hold | | | | |

HMW—high molecular weight species,
RP—relative potency,
MP—main peak,
LMW—low molecular weight species

### Example 5: Hold Time and Hold Temperature Dependence of the Decrease in HMW Levels of BITE Molecules after Storage Under Frozen Conditions

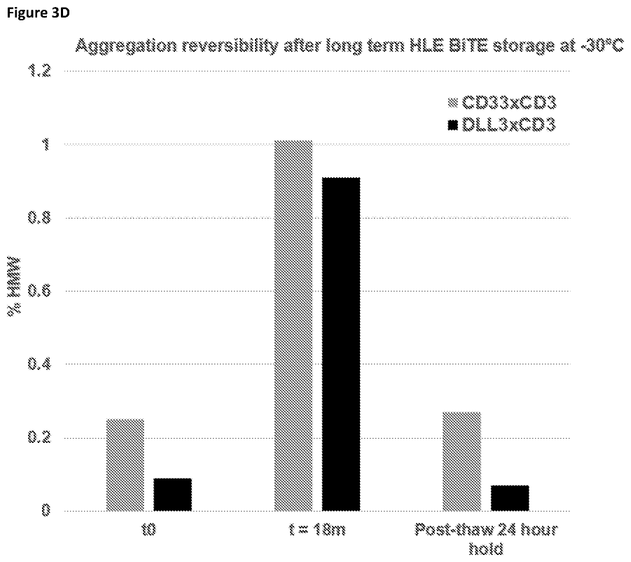

Compositions comprising 1 mg/mL each of the HLE BiTEs (CD33×CD3 and DLL3×CD3) and 2 mg/mL of a BiTE (EGFRvIII×CD3) were filled in 5 mL vials and stored at −20° C. for one month. After one month, the compositions were thawed at room temperature and analyzed immediately by size exclusion ultra high-performance liquid chromatography (SE-UHPLC) to determine the levels of HMW aggregates (e.g., bispecific antibody dimers) in the compositions. The thawed compositions were held at various temperatures for up to a maximum hold time of 96 hours and the HMW aggregate levels were analyzed at different time points by SE-UHPLC. The hold times required for the HMW aggregate levels to decrease to the initial pre-frozen levels for CD33×CD3 and DLL3×CD3 HLE BiTEs and EGFRvIII×CD3 BiTE at various temperatures were identical and is shown in FIG. 4.

### Example 6: Stabilizing Effect of Benzyl Alcohol (BA)

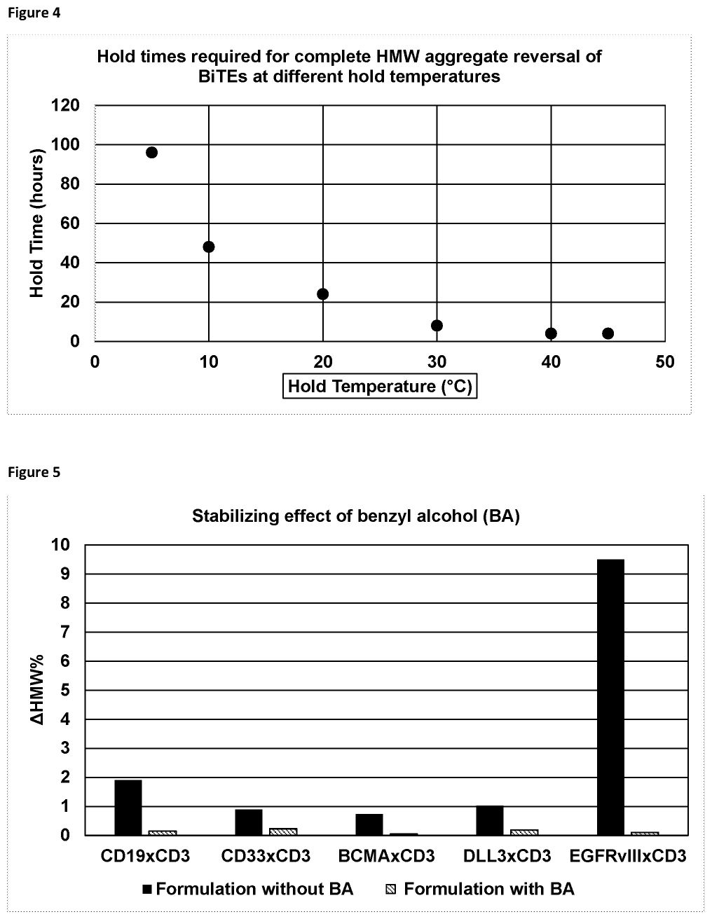

Compositions comprising 1 mg/mL of each of the HLE BiTEs (CD19×CD3, CD33×CD3, BCMA×CD3, DLL3×CD3, and EGFRvIII×CD3) were filled in 5 mL and stored at −20° C. in the presence and absence of BA for four weeks. After four weeks, the compositions were thawed at room temperature and analyzed immediately by SE-UHPLC to determine the levels of HMW aggregates. The presence of BA during the storage stabilizes the BiTEs (FIG. 5).

All references cited in this application are incorporated by reference herein

1. A method for reducing aggregates of a bispecific antibody comprising: holding a thawed bispecific antibody at a temperature from 5° C. to 45° C. for at least 4 hours, wherein the bispecific antibody has been stored at a temperature of from −20° C. to −40° C. before thaw.

2. The method of claim 1, wherein the bispecific antibody is a drug substance.

3. The method of claim 1, wherein the bispecific antibody is held at the temperature for a period of from 4 hours to 96 hours.

4. The method of claim 1, wherein the bispecific antibody is held at a temperature of from 10° C. to 30° C. for a period of from 8 to 48 hours.

5.-7. (canceled)

8. The method of claim 1, wherein the aggregates comprise high molecular weight (HMW) aggregates.

9. The method of claim 1, wherein the bispecific antibody comprises less than about 1% of the HMW aggregates after the holding period.

10. The method of claim 9, wherein the bispecific antibody comprises less than about 0.5% of the HMW aggregates.

11. (canceled)

12. A method for preparing a composition comprising a bispecific antibody, the method comprising
thawing a drug substance comprising a bispecific antibody that has been stored at a temperature of from −20° C. to −40° C., and
holding the thawed drug substance comprising the bispecific antibody at a temperature of from 5° C. to 45° C. for at least 4 hours.

13.-20. (canceled)

21. A method for preparing a composition comprising a bispecific antibody, the method comprises holding a thawed drug substance comprising a bispecific antibody at a temperature of from 5° C. to 45° C. for at least 4 hours, wherein the drug substance has been frozen at or above the glass transition temperature (Tg′) of the drug substance before the thaw.

22.-31. (canceled)

32. The method of claim 1, wherein the drug substance comprising the bispecific antibody at a concentration of from about 0.05 mg/mL to about 20 mg/mL.

33. The method of claim 32 further comprising lyophilizing the composition.

34. The method of claim 32 further comprising spray drying the composition.

35. The method of claim 34, wherein the bispecific antibody comprises
a first binding domain that binds to a target cell surface antigen, and
a second binding domain that binds to human CD3, and wherein the bispecific antibody is in (scFv)₂ format.

36. The method of claim 35, wherein the target cell surface antigen is CD19, CD33, or BCMA.

37. The method of claim 36, wherein the first binding domain comprises a VH region and VL region, wherein:
the VH comprises the amino acid sequence of SEQ ID NO: 77 and the VL comprises the amino acid sequence of SEQ ID NO: 78;
the VH comprises the amino acid sequence of SEQ ID NO: 28 and the VL comprises the amino acid sequence of SEQ ID NO: 32 or 33; or
the VH comprises the amino acid sequence of SEQ ID NO: 132 and the VL comprises the amino acid sequence of SEQ ID NO: 133.

38. The method of claim 37, wherein the bispecific antibody comprises the amino acid sequence of SEQ ID NO: 17, 40 or 135.

39. The method of claim 35, wherein the bispecific antibody further comprises a third domain comprising two polypeptide monomers, each comprises a hinge, a CH2 and a CH3 domain, wherein the two polypeptide monomers are linked to each other via a peptide linker.

40. The method of claim 39, wherein the third domain comprises in an amino to carboxyl order hinge-CH2-CH3-linker-hinge-CH2-CH3.

41. The method of claim 40, wherein the third domain is a HLE domain.

42. The method of claim 41, wherein the first binding domain binds to at least one target cell surface antigen selected from CD19, CD33, EGFRvIII, MSLN, CDH19, FLT3, DLL3, CDH3, CD70, BCMA or PSMA.

43. The method of claim 42, wherein the first binding domain comprises a VH region and a VL region, wherein
the VH comprises the amino acid sequence of SEQ ID NO: 108 and the VL comprises the amino acid sequence of SEQ ID NO: 109;
the VH comprises the amino acid sequence of SEQ ID NO: 27 and the VL comprises the amino acid sequence of SEQ ID NO: 32;
the VH comprises the amino acid sequence of SEQ ID NO: 48 and the VL comprises the amino acid sequence of SEQ ID NO: 49;
the VH comprises the amino acid sequence of SEQ ID NO: 59 and the VL comprises the amino acid sequence of SEQ ID NO: 60;
the VH comprises the amino acid sequence of SEQ ID NO: 77 and the VL comprises the amino acid sequence of SEQ ID NO: 78;
the VH comprises the amino acid sequence of SEQ ID NO: 108 and the VL comprises the amino acid sequence of SEQ ID NO: 112;
the VH comprises the amino acid sequence of SEQ ID NO: 89 and the VL comprises the amino acid sequence of SEQ ID NO: 90;
the VH comprises the amino acid sequence of SEQ ID NO: 100 and the VL comprises the amino acid sequence of SEQ ID NO: 101;
the VH comprises the amino acid sequence of SEQ ID NO: 121 and the VL comprises the amino acid sequence of SEQ ID NO: 122;
the VH comprises the amino acid sequence of SEQ ID NO: 188 and the VL comprises the amino acid sequence of SEQ ID NO: 189;
the VH comprises the amino acid sequence of SEQ ID NO: 132 and the VL comprises the amino acid sequence of SEQ ID NO: 133; or
the VH comprises the amino acid sequence of SEQ ID NO: 173 and the VL comprises the amino acid sequence of SEQ ID NO: 174.

44. The method of claim 43, wherein the bispecific antibody comprises the amino acid sequence of SEQ ID NO: 63, 114, 41, 82, 136, 104, 93, 177, 125, 190 or 52.

45. The method of claim 44, wherein the bispecific antibody consisting of the amino acid sequence of SEQ ID NO: 63, 114, 41, 82, 136, 104, 93, 177, 125, 190 or 52.

46. The method of claim 1, wherein the bispecific antibody is a bispecific masked antigen binding protein.

47. The method of claim 46, wherein the bispecific masked antigen binding protein comprises:
(a) a first antibody or antigen binding fragment thereof (AB1) that binds to a first antigen and a masking domain (MD1) coupled to AB1, wherein the MD1 comprises
(1) a first masking peptide (MP1) that inhibits or reduces the binding of AB1 to its antigen, and
(2) a protein recognition site (PR1), wherein binding to or cleavage of the PR1 by a protein or a protease increases AB1 binding to its antigen.
(b) a second antibody or antigen binding fragment there of (AB2) that binds to a second antigen, and a second masking domain (MD2) coupled to AB2, wherein the MD2 comprises:
(1) a second masking peptide (MP2) that inhibits or reduces the binding of AB2 to its antigen, and
(2) a second protein recognition site (PR2), wherein binding to or cleavage of the PR2 by a protein or protease increases AB2 binding to its antigen.

48.-49. (canceled)
